(19) United States
(12) Patent Application Publication
(10) Pub. No.: US 2022/0067249 A1
(43) Pub. Date: Mar. 3, 2022

(54) Machine Learning to Accelerate Design of Energetic Materials

(71) Applicant: Baldur Andrew Steingrimsson, Wilsonville, OR (US)

(72) Inventor: Baldur Andrew Steingrimsson, Portland, OR (US)

(73) Assignee: Imagars LLC, Wilsonville, OR (US)

(21) Appl. No.: 17/497,900

(22) Filed: Oct. 9, 2021

Related U.S. Application Data
(63) Continuation-in-part of application No. 16/782,829, filed on Feb. 5, 2020.
(60) Provisional application No. 63/189,209, filed on May 16, 2021.

(51) Int. Cl. G06F 30/27; G06F 30/28

(52) U.S. Cl. CPC G06F 30/27; G06F 2111/04; G06F 30/28

(57) ABSTRACT

This invention presents an innovative framework for the application of machine learning (ML) for identification of energetic materials with desired properties of interest. For the output properties of interest, we identify the corresponding driving (input) factors. We present a framework for a generic engine for predicting properties of energetic materials, once capable of interacting with and receiving support from physics-based prediction models, supporting joint optimization, accounting for properties both at macro- and micro-level, supporting multi-linear regression of descriptors, and offering physics-based interpretation of the descriptors. We present an approach for formulating descriptors, capable of both capturing properties and behavior of complex molecular structures, and that can be imported into ML algorithms for analysis. We show how combustion temperature and density can be analytically accounted for in a hybrid ML and physics-based model for optimizing a specific impulse, for purpose of making the most of the usually limited input data available.

### CROSS REFERENCE TO RELATED APPLICATIONS

Utility patent application Ser. No. 16/782,829, filed on Feb. 5, 2020, the entire contents of which are hereby incorporated by reference.

Provisional patent application No. 63/189,209, filed on May 16, 2021, the entire contents of which are hereby incorporated by reference.

### ACKNOWLEDGEMENT OF FEDERAL FUNDING

This utility patent traces its origin to research conducted under support of National Science Foundation Awards 1,447,395 and 1,632,408.

Baldur Steingrimsson very much appreciates the support from the National Science Foundation (IIP-1447395 and IIP-1632408), with the program directors, Dr. G. Larsen and R. Mehta. This invention was made with further Government support under Contract FA864921P0754 awarded by the U.S. Air Force and Contract N6833521C0420 awarded by the U.S. Navy. The Government has certain rights in the invention.

### BACKGROUND OF THE INVENTION

Energetic materials consist mainly of explosives, pyrotechnics, and propellants.

This patent addresses the application of data analytics and optimization techniques, such as machine learning, to accelerate the design of materials, exhibiting high energy density. Specifically, we present apparatus and methods for accelerating the design of energetic materials for propellants. The goal is to maximize the energy density, defined as energy produced per unit mass of propellant. In practical terms, this usually translates into optimization of a specific impulse, a density specific impulse, detonation pressure or detonation velocity. Similar apparatus and methods can be used for accelerating the design of energetic materials for explosives or pyrotechnics.

### 1. Technical Field Description

### 1.1 Definition and Basic Properties of Energetic Materials (High-Energy Density Compounds)

### 1. Definitions of High-Energy Density Compounds

High-energy-density material is a class of energetic materials, particularly propellants, with a high ratio of potential chemical energy output (releasable chemical energy stored) to density, a ratio usually referred to as the “thrust-to-weight ratio” (WikipediaHEDM 2021).

Quantitative criteria, for definition of HEDMs, involves both energy (density, detonation velocity and detonation pressure) and stability (bond dissociation energy of the trigger bond) considerations. The criteria of density of ≈1.9 g/cm³, detonation velocity of ≈9.0 km/sec, detonation pressure of ≈40.0 GPa and BDE of ≈80-120 kJ/mol has been used to expediently design and filtrate potential HEDC candidates (WangGongLiuDu 2011), (XiaoXuQiu 2008). These criteria are of great practical value for the design of composite explosives. In case of propellants, there is also a similar criterion involving the specific impulse (WangGongLiuDu 2011).

### 2. General Criteria for Selecting High-Energy Density Compounds (Propellants)

Propellants can provide energy for chemical rocket propelled systems by releasing chemical energy through a combustion process. They supply the working fluid that is expanded by the release of thermal energy for thrust. Propellants can also be burned in gas generators to drive turbopumps, hydraulic pumps or alternators inside a rocket.

Several factors need to be considered in the choice of a propellant, in particular

### 1. Performance;

2. Physical hazards; and
3. Economic factors.

The primary consideration for propellant selection relates to high performance coupled with low system weight. In addition, the physical and chemical characteristics of the propellants must meet the requirements of the operational environment expected. The need for instant readiness by the military also imposes demands for long term storability and ease of handling.

Performance refers here to the chemical energy content of the propellant and the resulting thrust (specific impulse) produced. Physical hazards refer to stability, storability and handling as well as to biological toxicity, corrosiveness and environmental impact. If a propellant is not very stable, then it may be difficult to store and handle and difficult to transport (the propellant may ignite upon minor change in environmental conditions). Propellant availability, manufacturing cost and logistic supply are accounted for as a part of the economic factors.

### 3. Derivative Structures of an Organic Molecule

The term derivative in chemistry usually refers to a compound that can be imagined to arise, or actually be synthesized, from a parent compound through replacement of one atom with another atom or a group of atoms. Derivatives have been used extensively in organic chemistry to assist with identification of compounds (ChemiCool 2021).

### 1.2 Energetic Materials for Space Applications

New energetic materials provide the opportunity to increase the operational capability of space-borne assets. FIG. 1-FIG. 4 present a systematic overview key categories of energetic materials, with emphasis on liquid rocket propellants (ForbesForrest 2003).

### 1. Energetic Materials in Context with the Basic Principles of Rocket Propulsion

Liquid rockets are reaction engines whose propulsion is governed by Newton's laws of motion, as shown in FIG. 5. Ship propellers, air plane propellers, water wheels and oars, in general, generate their forward push at the expense of the momentum of water, fuel or air masses which are accelerated towards the rear. The acceleration of a working fluid out of the rear of a vehicle produces thrust, shown in FIG. 5b. The working fluid may consist of cold gas, monopropellant decomposition products, bipropellant combustion products, or molecules that have been heated by an electrical, solar or nuclear energy source (or by other means). The thrust of a liquid propellant rocket is derived primarily from the high velocity of exhaust products exiting from the engine at the exit plane (ForbesForrest 2003).

### 2. Single-Mode Space Propulsion

Single-mode propulsion refers to a situation where separate propellants are used for fulfilling different types of propulsion requirements. Here, a single propellant is only used for a single propulsion mode. For single-mode propulsion, there typically may be either electric or chemical propellants used. Single-mode, chemical propellants can include mono- or bi-propellants.

Single-mode, chemical mono-propellants consist of single-source fuel, which, when ignited by ignition or a catalyst (something to spark it), result in combustion. In case of mono-propellants, the fuel is decomposed on a catalyst in a combustion chamber, as shown in FIG. 6, but without an oxidizer, resulting in gases coming out. And there is a nozzle attached to it, so a thrust is generated. Many smaller satellites just use hydrazine in a catalyst for propulsion. For a listing of mono-propellants, such as hypergolic or mixed amine propellants, refer to (ForbesForrest 2003).

Single-mode, chemical bi-propellants are made up of two separate propellants, a fuel and an oxidizer. The fuel and the oxidizer are fed separately to a thrust chamber, where mixing and combustion occurs (the fuel gets oxidized with the oxidizer, with or without an external ignition source, as shown in FIG. 7), resulting in an overall thrust. Mono-methyl hydrazine, CH₃(NH)NH₂, is a volatile hydrazine chemical, which is used as propellant in bipropellant rocket engines, because of its hypergolic properties, when combined with oxidizers such as nitrogen tetroxide (N₂O₄) or nitric acid (HNO₃). For further information, refer to military specification MIL-PRF-27404.

Single-mode electrical propulsion includes Hall-effect thrusters and electro-sprays. In a Hall-effect thruster, the propellant is decomposed, ionized, and the ions are extracted. Hall-effect thrusters use a magnetic field to limit the electrons' axial motion and then use them to ionize the propellant, efficiently accelerating the ions to produce thrust. Hall thrusters operate on a variety of propellants, the most popular being xenon and krypton. In an electro-spray space application, the propellant is sprayed in high electric field, resulting in thrust (EdgarChoueiri 2009).

### 3. Multi-Mode Space Propulsion

Multi-mode propulsion refers to a situation where a single propellant is capable of fulfilling multiple propulsion requirements. For example, one ideally would like to use the same propellant to launch a satellite system and to interject a satellite into orbit and to do orbit maneuvering and to move the satellite to a different orbit. But so far, this has not been possible (this has come across as a wish list). Hence, one has had to break it down. In case of space propulsion, only a handful of techniques are available for multi-mode propulsion, mainly electric propulsion or chemical propulsion, both of which can have their own sub-modes associated, as noted above.

Multi-mode propulsion seeks to simplify the propellant logistics. But this may call for identification of special properties. Propellant design for multi-mode aims at discovery of a single propellant for fulfilling all these functions (requirements) in space. The exact implementation may depend on the specific mission requirements. Even if a single propellant could support two modes, but not necessarily all of the modes, it already may be considered a significant achievement.

### 1.3 Energetic Materials for Underwater or Land-Based Applications

### 1. Propellants

Within the realm of propellants for space applications, there are propellants, that can be used for underwater or land-based applications, possibly with some adaptations. But traditionally, there tend to be unique requirements for these applications, which may or may not overlap with the requirements for space applications.

### 2. Explosives

The development of next-generation energetic materials with improved detonation performance is of significant importance for applications involving civilian or military use. High-explosive detonations are violent processes with high-energy release, that at first appearance may seem difficult to predict and control. Yet, they involve well-defined physical and chemical phenomena.

There is a fairly high degree of resemblance between the design objectives for propellants and for explosives. To a chemist pursuing design of new high-explosive compounds, the ability to compute detonation properties (detonation pressure, energy and velocity as well as product composition), from a given molecular structure and the known or estimated crystal density, is of great importance. The calculated properties are likely to highly impact the decision as to whether it is worth the effort to attempt a new, complex, costly and time-consuming synthesis of candidate compounds (KamletJacobs 1968).

The detonation waves resulting from explosives can be described approximately by Chapman-Jouguet theory of detonation. The Chapman-Jouguet condition states that the detonation propagates at a velocity at which the reacting gases just reach sonic velocity (in the frame of the leading shock wave) as the reaction ceases (ChapmanJouguet 2021).

### 3. Pyrotechnics

Similar to the explosives, the pyrotechnics involve quick release of large amounts of gaseous products through controlled chemical reactions. Solid propelling species were first used by the Chinese some 2000 years ago, as fireworks. The pyrotechnic behavior of solid propellants can be characterized by (1) an evaluation of their various modes of decomposition, and (2) a knowledge of their threshold and type of reaction (JacquesBrunet 1993).

### 1.4 Application of AI or ML to the Design of Energetic Materials

Data-driven approaches provide new opportunities to accelerate the discovery of such energetic materials, through rapid screening of material property data sets, per FIG. 8. Such approaches can both accelerate the identification of propellants with greater energy density and new energetic molecules.

Traditionally, the development of an energetic material has primarily been based on test-driven approaches, and as such, the continued application of such methodologies may not be cost-effective, sustainable, or sufficiently responsive to warfighter needs. Over the past few decades, chemists have synthesized a large number of energetic materials and generated their corresponding physio-chemical data sets. In many cases, the actual materials have not been considered beyond their laboratory characterization, and systematic efforts of using resulting data sets to learn and accelerate new material discoveries have not been undertaken within the energetic material community.

To this end, AI, ML or DL techniques may be considered promising approaches, for utilizing available energetic material data sets, and for providing design rules for better and faster property predictions. AI, ML or DL techniques can be used as aids for purpose of guiding decisions related to propellant selection. One subset of techniques, referred to as sequential learning, combines data-driven ML approaches with experimentation, as shown in FIG. 8, for purpose of necessitating fewer experiments and decreasing cost. In general, methods from this suite take advantage of data that resides in current databases to greatly accelerate the discovery of new energetic molecules, since it greatly reduces the amount of computationally intensive brute force calculations used to calculate molecular properties from first principles or correlative techniques. FIG. 9 provides a high-level overview of the key underlying, correlative relationships, in case of energetic materials. FIG. 10 provides a more detailed overview of the underlying relationships that apply to propellants.

### DESCRIPTION OF PRIOR ART

### 1. Selected Prior Art on Materials with High Energy Density

Ref (ForbesForrest 2003) provides tutorial overview of liquid rocket propellants, both of the fundamental theory of rocket propulsion and the key propellant categories.

In (KokanOldsSeitzman 2009), a technique for computationally determining the thermophysical properties of HDEM propellants is presented. For purpose of accurately modeling rocket engine performance, cost and weight in a conceptual design environment, several thermodynamic and physical properties are required over a range of temperatures and pressure. The approach presented combines quantum mechanical and molecular dynamic calculations and group additivity methods. The authors note that optimization of the energy density of a chemical propellant may only address a part of the problem, in the case of multi-mode propulsion.

Ref (RoveyLyneMundahl 2020) presents a review of multi-mode space propulsion. A review of the numerous multi-mode concepts, that have been explored and documented in the literature, is presented. This includes the concepts of combining cold gas, monopropellant, bipropellant and solid chemical propulsion with electrothermal, electrostatic or electromagnetic electric propulsion. A review of the nature of multi-mode propulsion, mission analyses, benefits and specific multi-mode concepts is also presented.

### 2. Prior Art on Applications of ML and Data Analytics to Design of Energetic Materials

While state-of-the-art AI and ML techniques have been reported in the literature, and widely used in other commercial areas, such as in image processing, target recognition, social networking, and health or financial sectors, they have not been applied extensively yet to the design of energetic materials for space propulsion or space access applications.

In (KangLiu 2020), ML, MI, and thermochemical data are combined to screen prospective candidates of energetic materials. To directly characterize energetic performance, the heat of explosion ΔHₑ is employed as the primary property of interest (target property). The critical descriptors of cohesive energy, averaged over all constituent elements and the oxygen balance, are determined by forward stepwise selection from a large number of possible descriptors. With them and a theoretically labeled ΔHₑ training data set, a satisfactory surrogate ML model has been trained. The ML model is then applied to large databases from ICSD and PubChem to predict ΔHₑ. At the gross-level filtering by the ML model, 2,732 molecular candidates consisting of CHNO with high ΔHₑ values are predicted. Afterwards, a fine-level thermochemical screening is conducted on the 2,732 materials, resulting in 262 candidates with TNT equivalent power index P_{e(TNT)} greater than 1.5. By raising P_{e(TNT)} further to larger than 1.8, 29 prospective candidates of high energetic materials (CHNO-based molecules) are identified from the 2,732 materials considered. All of these candidates are new to the reservoir of well-known energetic materials.

According to (YuanZhangTao 2020), a new generation of rocket propellants for deep space exploration, ionic liquid propellants, with long endurance and high stability, is attracting more and more attention. However, a major defect of ionic liquid propellants, that restricts their application, entails inadequate hypergolic reactivity between the fuel and the oxidant. This defect can result in local burnout and accidental explosions during the launch process. In (YuanZhangTao 2020), the authors propose a visualization model to show the features of structure, density, thermal stability, and hypergolic activity for estimating propellant performances and their application abilities. The authors believe the propellant materials genome and visualization model proposed will greatly improve the efficiency and quality of developing high-performance propellants, which may benefit the discovery of new advanced functional molecules in the field of energetic materials.

The authors of (HuangLiTanWen 2021) applied ML to the study of a long-standing performance-stability contradiction involving HEDMs. The authors provided optimal ranges of critical features, such as for molecular weight (>260 Da), molecular backbone shape (bridged), oxygen balance (−60%-0%), material density (>1.73 g/cm³) and the amount and strength of hydrogen bonds (2.5 kcal/mol-5 kcal/mol), that favor improvement of both detonation and stability of HEDMs. More specifically, the authors tailored a scheme for predicting five (5) quantities for the HEDM candidates considered, i.e., detonation velocity, detonation pressure, heat of explosion, decomposition temperature, and lattice energy. ML is employed to handle 28 feature descriptors and 5 properties of detonation and stability of 153 HEDMs. All the data points used, 21,648 in number, have been obtained through high-throughput crystal-level quantum mechanics calculations on supercomputers. Five models are considered, namely, XGBoost, adaptive boosting, random forest, multilayer perceptron and kernel ridge regression. These models have been trained and evaluated by stratified sampling and a 5-fold cross-validation method. Among these models, a XGBoost model produced the best scoring metrics in predicting the detonation velocity, detonation pressure, heat of explosion, decomposition temperature, and lattice energy of HEDMs. XGBoost predictions have provided the best agreement with 1,383 experimental data points extracted from the literature.

### 3. Prior Art on Applications of ML and Data Analytics to Design of Other Materials

For information on ML and data analytics to accelerate the design of alloys, refer to (SteingrimssonFanKulkarni 2020). Ref. (SteingrimssonFanKulkarni 2021) contains additional information on the use of ML and data analytics to accelerate the design of high-entropy materials as well as the associated manufacturing processes.

### 4. Prior Art on Explosives

For prior art on explosives, refer to Ref (jpAgrawal 1998), Ref (AgrawalSurveSonawane 2000), Ref (BadgujarTalawarAsthana 2008), Ref (BauerBenzKlapotke 2021), Ref. (DuQuWangCuiWang 2021), Ref. (EatonGilardiZhang 2000), Ref (FanJu 2008), Ref (FershtatMakhova 2020), Ref. (GaoLiuSuZhangYang 2014), Ref (GeithKlapotkeWeigand 2004), Ref (GutowskiRogersDixon 2007), Ref (HaoLiuZhaiQiuMa 2020), Ref (HenryParkinsonMayer 2001), Ref (HuangTangImlerParrish 2020), Ref (Koch 2015), Ref (LiGuoLiSong 2009), Ref. (LiJuZhang 2017), Ref. (LiaoJuZhaoYi 2010), Ref. (LiuWangWangGong 2013), Ref. (McDonaldBennionLeone 2016), Ref (McDonaldSethMatzger 2015), Ref. (NairAsthanaRaoGandbe 2010), Ref (QiuXiaoGongJuZhu 2006), Ref (QiuXiaoZhuXiaoGong 2006), Ref. (RaviGoreTewariSikder 2012), Ref. (SethMatzger 2017), Ref. (TalawarSivabalan Mukundan 2009), Ref (TanLuLiuYan 2021), Ref (TangKumarShreeve 2017), Ref. (TappanBrill 2003), Ref (ThottempudiGaoShreeve 2011), Ref (ThottempudiShreeve 2011), Ref. (WangGongLiuXiao 2010), Ref. (WangGongXiao 2009), Ref (WuLiHuZhangZhu 2020a), Ref. (WuLiHuZhangZhu 2020b), Ref (YangWangGongZhangWang 2018), Ref. (YinShreeve 2015), Ref (YongjinShuhong 2019), Ref. (YuYinZhangHeImler 2017), Ref (YuanGanJiangZhu 2021), Ref (ZhangShreeve 2014), Ref. (ZhangDuWangGongHuang 2011), Ref (ZhangZhangDengQi 2017), Ref. (ZhangZhuXiao 2010), Ref (ZhangWangJiZhouZhou 2008), Ref (ZhaoLu 2012a), Ref (ZhaoLu 2013a) and Ref (ZhaoLu 2013b).

### 5. Other Prior Art

While patents have been issued on rocket engines, and on the flow of propellants through such engines, much less is available on the optimization of energetic materials (propellants), using machine learning, data analytics or similar techniques. Ref. (CarrHacohenWu 2019), titled Improved HLA Epitope Prediction, may be the closest analogue (in the sense of presenting use of machine learning for an application involving some type of chemistry or genomics). The authors of Ref (CarrHacohenWu 2019) discovered new binding motifs, established the role of gene expression in peptide presentation and improved prediction of human leukocyte antigen-peptide binding by using these data to train machine learning models. The streamlined experimental and analytic workflows considered enable direct identification and analysis of endogenously processed and presented antigens. Along similar lines, Ref. (WestNevins 2009) introduces binary prediction tree modeling with many predictors and its uses in clinical and genomic applications. Ref (AndersonXieWu 2019) presents a Petroleum Analytics Learning Machine system, which is a machine learning based, “brutally empirical” analysis system for use in all upstream and midstream oil and gas operations. The PALM system optimizes exploration, production and gathering from at least one well of oil and natural gas fields to maximize production while minimizing costs.

Ref (SpanjersSchilling 2004) presents description of methods to increase propellant throughput in a micro-pulsed plasma thruster. Ref (KeidarShashurinZhuang 2016) introduces a micro-cathode thruster together with a method for increasing thrust output for a micro-cathode thruster. Ref. (KeithCoste 2010) depicts a catalytically activated transient decomposition propulsion system, one that provides thrust by decomposing flow-controlled propellant in contact with a catalyzing agent using a fixed volume of liquid propellant that is placed in contact with the catalyst within the decomposition chamber by a calibrated flow control valve. Ref. (PedersonSchmotolocha 2010) describes a compact, high performance combustion rocket engine system with a swirl generator.

### REFERENCES

(WikipediaHEDM 2021) Wikipedia, “High-energy-density matter”, https://en.wikipedia. org/wiki/High-energy-density_matter, Accessed on May 5, 2021.
(WikipediaEnthalpyFormation 2021) Wikipedia, “Standard Enthalpy of Formation”, https://en.wikipedia.org/wiki/Standard_enthalpy_of_formation, Accessed on May 12, 2021.
(WikipediaOB 2021) Wikipedia, “Oxygen Balance”, https://en.wikipedia.org/wiki/Oxygen_balance, Jul. 11, 2021.
(WikipediaAromaticity 2021) Wikipedia, “Aromaticity”, https://en.wikipedia.org/wiki/Aromaticity, Accessed on May 14, 2021.
(WikipediaGroupAdditivity 2021) Wikipedia, “Benson Group Increment Theory”, https://en.wikipedia.org/wiki/Benson_group_increment_theory, Accessed on Jul. 3, 2021.
(WikipediaReaxFF 2021) Wikipedia, “ReaxFF”, https://en.wikipedia.org/wiki/ReaxFF, Accessed on Jul. 11, 2021.
(HeatOfFormationAdditivity 2021), Wikipedia, “Heat of Formation Group Additivity”, https://en.wikipedia.org/wiki/Heat_of_formation_group_additivity, Accessed on Jul. 4, 2021.
(WikipediaGaucheEffect 2021) Wikipedia, “Gauche Effect”, https://en.wikipedia.org/wiki/Gauche_effect, Accessed on Jul. 4, 2021.
(WikipediaNWChem 2021) Wikipedia, “NWChem”, https://en.wikipedia.org/wiki/NWChem/, Accessed on Jul. 18, 2021.
(PentaneInterference 2021) Wikipedia, “Pentane Interference”, https://en.wikipedia.org/wiki/Pentane_interference, Accessed on Jul. 4, 2021.
(WikipediaPareto 2021) Wikipedia, “Pareto Efficiency”, https://en.wikipedia.org/wiki/Pareto_efficiency, Accessed on Jul. 17, 2021.
(WikipediaTurbineBlade 2021) Wikipedia, “Turbine Blade”, https://en.wikipedia.org/wiki/Turbine_blade, Accessed on Aug. 14, 2021.
(WikipediaDirectional Solidification 2021) Wikipedia, “Directional Solidification”, https://en.wikipedia.org/wiki/Directional_solidification, Accessed on Aug. 14, 2021.
(WikipediaSingleCrystal 2021) Wikipedia, “Single Crystal”, https://en.wikipedia.org/wiki/Single_crystal, Accessed on Aug. 14, 2021.
(WikipediaScheilEquation 2021) Wikipedia, “Scheil Equation”, https://en.wikipedia.org/wiki/Scheil_equation, Accessed on Sep. 12, 2021.
(HenryCurran 2019) Henry Curran, “Chemical Kinetic Modelling for Combustion”, https://cefrc.princeton.edu/sites/cefrc/files/day1_curran.pdf, Accessed on Jul. 3, 2021.
(MolecularKnowledge 2021) Molecular Knowledge Systems, “Designing Better Chemical Products”, http://www.molknow.com/Online/Estimation.htm, Accessed on Jul. 3, 2021.
(ChemiCool 2021) Chemicool Dictionary, “What is a Derivative in Chemistry”, https://www. chemicool.com/definition/derivative.html , Accessed on May 5, 2021.
(ForbesForrest 2003) Forrest Forbes and Peter Splinter, “Liquid Rocket Propellants”, Encyclopedia of Physical Science and Technology (Third Edition), pp. 741 777, https://doi.org/10.1016/B0-12-227410-5/00385-9, 2003.
(EdgarChoueiri 2009) Edgar Y. Choueiri, “New Dawn for Electric Rockets”, Scientific American, Vol. 300, No. 2, pp. 58-65, 2009.
(KokanOldsSeitzman 2009) T. S. Kokan, J. R. Olds, J. M. Seitzman, and P. J. Ludovice, “Characterizing high-energy-density propellants for space propulsion applications”, Acta Astronautica, Vol. 65, No. 7-8, pp. 967-986, October-November 2009.
(RoveyLyneMundahl 2020) J. L. Rovey, C. T. Lyne, A. J. Mundahl, N. Rasmont, M. S. Glascock, M. J. Wainwright, and S. P. Berg, “Review of multimode space propulsion”, Progress in Aerospace Sciences, Vol. 118, https://doi.org/10.1016/j.paerosci.2020.100627, 2020.
(SteingrimssonFanKulkarni 2020) B. Steingrimsson, X. Fan, A. Kulkarni, D. Kim and P. K. Liaw, “Machine Learning to Accelerate Alloy Design”. U.S. patent application Ser. No. 16/782,829, https://patents.google.com/patent/US20200257933A1, filed on Feb. 5, 2020.
(SteingrimssonFanKulkarni 2021) B. Steingrimsson, X. Fan, A. Kulkarni, M. C. Gao and P. K. Liaw, “Machine Learning and Data Analytics for Design and Manufacturing of High-Entropy Materials Exhibiting Mechanical or Fatigue Properties of Interest,” in “Fundamental Studies in High-Entropy Materials”, https://arxiv.org/abs/2012,07583, Springer, Editors: Dr. Peter K. Liaw and Dr. James Brechtl, 2021.
(SteingrimssonKulkarni 2020) B. Steingrimsson and A. Kulkarni, Automatic Requirement Verification Engine and Analytics, U.S. Pat. No. 10,853,536, https://patents.google.com/patent/US10853536B1/, granted on Dec. 1, 2020.
(SteingrimssonFanYang 2021) B. Steingrimsson, X. Fan, X. Yang, M.O Gao, Y. Zhang and P. K. Liaw, “Predicting Temperature-Dependent Ultimate Strengths of BCC High-Entropy Alloys”, vol. 7, no. 152, DOI 10.1038/s41524-021-00623-4, https://www.nature.com/articles/s41524-021-00623-4.pdf, published in NPJ Computational Materials on Sep. 24, 2021.
(JacquesBrunet 1993) Jacques Brunet, “Chapter 7 Safety Characteristics of Solid Propellants and Hazards of Solid Rocket Motors”, in Solid Rocket Propulsion Technology, pages 303-327, https://doi.org/10,1016/B978-0-08-040999-3.50012-6, 1993.
(LlnlCheetah 2021) Cheetah: A next generation thermochemical code, https://www.osti. gov.biblio/95184, https://doi.org/10.2172/95184, accessed on Jun. 19, 2021.
(LlnlCheetah9.0 2021) Lawrence Livermore National Laboratory, Cheetah 9.0, https://pls. llnl.gov/people/divisions/materials-science-division/cheetah, accessed on Jun. 19, 2021.
(CheetahNextGen 2021) L. Fried and P. Souers, Cheetah: A Next Generation Thermochemical Code, https://www.osti.gov/servlets/purl/95184, accessed on Jun. 19, 2021.
(NasaGlennCEA 2021) NASA Glenn Research Center, “Chemical Equilibrium with Applications”, https://www1.grc.nasa.gov/research-and-engineering/ceaweb/, accessed on Jun. 19, 2021.
(CeaUserManual 2021) NASA NTRS, “Computer Program for Calculation of Complex Chemical Equilibrium Compositions and Applications II. Users Manual and Program Description, https://ntrs.nasa.gov/citations/19960044559, accessed on Aug. 28, 2021.
(SwriNpss 2021), SouthWest Research Institute, Numerical Propulsion System Simulation (NPSS), https://www.swri.org/consortia/numerical-propulsion-system-simulation-npss, accessed on Jun. 19, 2021.
(HallRascheSimons 2006) E. J. Hall, J. Rasche, T. A. Simons and D. Hoyniak, “NPSS Multidisciplinary Integration and Analysis,” https://ntrs.nasa.gov/citations/20060009942, prepared under Contract NAS3-98003, Task 5, Cleveland, Ohio, 2006.
(BradfordCharania 2004) J. E. Bradford, A. C. Charania and B. St. Germain, “REDTOP2: Rocket Engine Design Tool Featuring Engine Performance, Weight, Cost, and Reliability”, 40^{th} AIAA/ASME/SAE/ASEE Joint Propulsion Conference and Exhibit, Jul. 11-14 2004, Fort Lauderdale Fla., https://doi.org/10.2514/6.2004-3514, 2004.
(vanDuinDasgupta 2001) A. C. T. van Duin, S. Dasgupta, F. Lorant and W. A. Goddard, “ReaxFF: A Reactive Force Field for Hydrocarbons”, J. Phys. Chem. A, Vol. 105, No. 41, pp. 9396-9409, https://doi.org/10.1021/jp004368u, 2001.
(ShanThompson 2013) R. Shan, A. Thompson and S. Plimpton, “Large-Scale Molecular Dynamics Simulations with LAMMPS”, Computational Materials Science Initiative International Symposium, Nagoya, Japan, https://www.osti.gov/servlets/purl/1115164, 2013.
(JonesZhouLammps 2021) R. Jones, X. Zhou, S. Plimpton and A. Thomson, “Capabilities LAMMPS: Open-Source, High-Performance, and High-Fidelity Molecular Dynamics Code for Simulations of Chemical and Physical Processes of Materials”, https://h2awsm.org/capabilities/lammps-open-source-high-performance-and-high-fidelity-molecular-dynamics-code, accessed on Jun. 27, 2021.
(LammpsLibraryInterfaces 2021) LAMMPS Library Interfaces, https://docs.lammps.org/Library.html, accessed on Aug. 28, 2021.
(GuoGuo 2018) D. Guo, D. Guo, F. Huang and Q. An, “Influence of Silicon on the Detonation Performance of Energetic Materials from First-Principles Molecular Dynamics Simulations”, J. Phys. Chem. C, Vol. 122, No. 42, pp. 24,481-24,487, https://doi.org/10.1021/acs.jpcc.8b08305, 2018.
(CarrHacohenWu 2019) Steven A. Carr, Nir Hacohen, Catherine J. Wu et al., “Improved HLA Epitope Prediction”, Pub. No. US 2019/0346442 A1, https://patents.google.com/patent/US20190346442A1/, published on Nov. 14, 2019.
(AndersonXieWu 2019) Roger N. Anderson, Boyi Xie, Leon L. Wu, Arthur Kressner, “Petroleum Analytics Learning Machine System with Machine Learning Analytics Applications for Upstream and Midstream Oil and Gas Industry”, U.S. Pat. No. 10,430,725 B2, https://patents. google.com/patent/US10430725B2/, issued on Oct. 1, 2019.
(SpanjersSchilling 2004) Gregory G. Spanjers, John Schilling, David White, “Description of Methods to Increase Propellant throughput in a Micro Pulsed Plasma Thruster”, U.S. Pat. No. 6,769,241 B2, https://patents.google.com/patent/US6769241B2, issued on Aug. 3, 2004.
(KeidarShashurinZhuang 2016) Michael Keidar, Alexey Shashurin and Tai Sen Zhuang, “Micro-Cathode Thruster and a Method of Increasing Thrust Output for a Micro-Cathode Thruster”, U.S. Pat. No. 9,517,847 B2, https://patents.google.com/patent/US9517847B2, issued on Dec. 13, 2016.
(WestNevins 2009) Mike West and Joseph R. Nevins, “Binary Prediction Tree Modeling with Many Predictors and Its Uses in Clinical and Genomic Applications”, US Patent Pub. No. US 2009/0319244 A1, https://patents.google.com/patent/TW200415524A/, Dec. 24, 2009.
(KeithCoste 2010) Keith Coste, “Catalytically Activated Transient Decomposition Propulsion System”, U.S. Pat. No. 7,757,476 B2, https://patents.google.com/patent/US7757476B2/, issued on Jul. 20, 2010.
(PedersonSchmotolocha 2010) Robert J. Pederson and Stephen N. Schmotolocha, “Compact, High Performance Swirl Combustion Rocket Engine”, U.S. Pat. No. 7,690,192 B2, https://patents.google.com/patent/US7690192B2/, issued on Apr. 6, 2010.
(ChapmanJouguet 2021) Wikipedia, “ChapmanJouguet condition”, https://en.wikipedia.org/wiki/Chapman-Jouguet_condition, accessed on Jun. 18, 2021.
(JANNAF 2021) How to Order, Joint Army Navy NASA Air Force Interagency Propulsion Committee, https://www.jannaf.org/howtoorder, 2021.
(JannafThermoChem 2021) GitHub, “ThermoChem”, https://github.com/adelq/thermochem/blob/master/thermochem/janaf.py, Accessed on May 5, 2021.
(PubChemDatabaseAPI 2021) PubChem Docs, “Programmatic Access”, https://pubchemdocs.ncbi.nlm.nih.gov/programmatic-access, Accessed on May 5, 2021.
(ICSDdatabaseAPI 2021) ICSD, ICSD Products, https://icsd.products.fiz-karlsruhe.de/en/products/icsd-products, Accessed on May 5, 2021.
(NISTdatabase 2021) NIST Chemistry WebBook, NIST Standard Reference Database Number 69, https://webbook.nist.gov/chemistry/, DOI: https://doi.org/10.18434/T4D303, Accessed on Jun. 16, 2021.
(NISTwebBook 2021) NIST Chemistry WebBook, SRD 69, “Group Additivity Based Estimates”, https://webbook.nist.gov/chemistry/grp-add, Accessed on Jul. 3, 2021.
(NISTpotentialRepository 2021) NIST Interatomic Potentials Repository, Overview, https://www.ctcms.nist.gov/potentials/, Accessed on Jun. 28, 2021.
(QuantumChemistrySW 2021) Wikipedia, “List of quantum chemistry and solid-state physics software”, https://en.wikipedia.org/wiki/List_of_quantum_chemistry_and_solid-state_physics_software/, Accessed on Jun. 21, 2021.
(MatlabMachineLearning 2021) MathWorks, “Statistics and Machine Learning Toolbox”, https://www.mathworks.com/products/statistics.html, Accessed on May 13, 2021.
(MatlabDeepLearning 2021) MathWorks, “Deep Learning Toolbox”, https://www. mathworks.com/products/deep-learning.html, Accessed on May 13, 2021.
(MatlabReinforcementLearning 2021) MathWorks, “Reinforcement Learning Toolbox”, https://www.mathworks.com/products/reinforcement-learning.html, Accessed on May 13, 2021.
(MatlabDeepLearningHDL 2021) MathWorks, “Deep Learning HDL Toolbox”, https://www.mathworks.com/products/deep-learning-hdl.html, Accessed on May 13, 2021.
(PythonKeras 2021) Python Keras, “Deep Learning for Humans”, https://keras.io/, Accessed on May 13, 2021.
(PythonNumPy 2021) NumPy, “The fundamental package for scientific computing with Python”, https://numpy.org/, Accessed on May 13, 2021.
(PythonSciKitLearn 2021) SciKit-Learn, “Machine Learning in Python”, https://scikit-learn.org/, Accessed on May 13, 2021.
(PythonCaffe 2021) Caffee, “Deep learning framework by BAIR”, https://caffe. berkeleyvision.org/, Accessed on May 13, 2021.
(PythonTorch 2021) PyTorch, “An open source machine learning framework that accelerates the path from research prototyping to production deployment”, https://pytorch.org/, Accessed on May 13, 2021.
(PyQLearning 2021) pyqlearning 1.2.5, “pyqlearning is Python library to implement Reinforcement Learning and Deep Reinforcement Learning”, https://pypi.org/project/pyqlearning/, Accessed on May 13, 2021.
(PyTensorForce 2021) GitHub, “Tensorforce: A TensorFlow library for applied reinforcement learning”, https://github.com/tensorforce/tensorforce, Accessed on May 13, 2021.
(PyRLcoach 2021) rl-coach 1.0.1, “Reinforcement Learning Coach enables easy experimentation with state of the art Reinforcement Learning algorithms”, https://pypi.org/project/rl-coach/, Accessed on May 13, 2021.
(Pakdehi 2017) S. G. Pakdehi, M. H. Keshavarz, M. Akbari, M. Ghorbani, “Assessment of Physico-Thermal Properties, Combustion Performance and Ignition Delay Time of Dimethyl Amino Ethanol as Novel Liquid Fuel”, Propellants, Explosives, Pyrotechnics, vol. 42, no. 4, pp. 423-429, https://doi.org/10.1002/prep.201600113, 2017.
(KangLiu 2020) Peng Kang, Zhongli Liu, Hakima Abou-Rachid and Hong Guo, “Machine-Learning Assisted Screening of Energetic Materials”, The Journal of Physical Chemistry virtual special issue on “Machine Learning in Physical Chemistry”, Vol. 124, pp. 5341-5351, 2020.
(DouglasRapp 1990) Douglas C. Rapp, “High Energy-Density Liquid Rocket Fuel Performance”, NASA Contractor Report 185279, 26^{th} Joint Propulsion Conference, AIAA-90-1968, https://doi.org/10.2514/6.1990-1968, July 1990.
(GagneMcDevittHitt 2018) Kevin R. Gagne, M. Ryan McDevitt and Darren L. Hitt, “A Dual Mode Propulsion System for Small Satellite Applications”, vol. 5, no. 2, https://doi.org/10.3390/aerospace5020052, Aerospace, 2018.
(Türker 2013) Lemi Turker, “A Treatise on Impulse Values of Pure Explosives”, Journal of Theoretical & Computational Science, vol. 1, no. 1, doi: 10.4172/2376-130X.1000101, 2013.
(YuanZhangTao 2020) Wen-Li Yuan, Lei Zhang, Guo-Hong Tao, Shuang-Long Wang, You Wang, Qiu-Hong Zhu, Guo-Hao Zhang, Zhang Zhang, Ying Xue, Song Qin, Ling Hi, Jean′ne M. Shreeve, “Designing high-performance hypergolic propellants based on materials genome”, Science Advances, Vol. 6, No. 49, https://doi.org/10.1126/sciadv.abb1899, 2020.
(WangXuZhang 2019) Guixiang Wang, Yimin Xu, Wenjing Zhang, Chuang Xue, and Xuedong Gong, “Theoretical study on polyglycerine polynitrates for potential high-energy plasticizers of propellants”, Canadian Journal of Chemistry, Vol. 97, No. 4, April 2019.
(DuHanLiuWu 2019) Mingran Du, Tifei Han, Feng Liu and Hongbo Wu, “Theoretical investigation of the structure, detonation properties, and stability of bicyclo[3.2.1]octane derivatives”, Journal of Molecular Modeling, vol. 25, No. 253, https://doi.org/10.1007/s00894-019-4116-2, 2019.
(WangGongLiuDu 2011) Gui-Xiang Wang, Xue-Dong Gong, Yan Liu, Hong-Chen Du, Xiao-Juan Xu, He-Ming Xiao, “Looking for High Energy Density Compounds Applicable for Propellant among the Derivatives of DPO with —N₃, —ONO₂, and —NNO₂ Groups”, Computational Chemistry, Vol. 32, No. 5, pp. 943-952, https://doi.org/10.1002/jcc.21679, 2011.
(SavoskinKapkan 2007) M. V. Savos'kin, L. M. Kapkan, G. E. Vaiman, A. N. Vdovichenko, O. A. Gorkunenko, A. P. Yaroshenko, A. F. Popov, A. N. Mashchenko, V. A. Tkachev, M. L. Voloshin & Yu. F. Potapov, “New approaches to the development of high-performance hydrocarbon propellants”, Russian Journal of Applied Chemistry, Vol. 80, pp. 31-37, 2007.
(PoggioGirosi 1998) T. Poggio and F. Girosi, “A Sparse Representation for Function Approximation,” Neural Computation, vol. 10, no. 6, p. 1445-1454, 1998.
(JingLiWangShu 2014) Mei Jing, Huarong Li, Jun Wang, Yuanjie Shu, Xiaoyu Zhang, Qing Ma and Yigang Huang, “Theoretical investigation on the structure and performance of N, N′-azobis-polynitrodiazoles”, Journal of Molecular Modeling, Vol. 20, No. 2155, https://doi.org/10.1007/s00894-014-2155-2, 2014.
(LinChenZhuZhang 2013) Hₑ Lin & Peng-Yuan Chen & Shun-Guan Zhu & Lin Zhang, Xin-Hua Peng & Kun Li & Hong-Zhen Li, “Theoretical studies on the thermodynamic properties, densities, detonation properties, and pyrolysis mechanisms of tri nitromethyl-substituted aminotetrazole compounds”, Vol. 19, pp. 2413-2422, https://doi.org/10.1007/s00894-013-1783-2, 2013.
(QiuXiaoGongJu 2007) Ling Qiu, Heming Xiao, Xuedong Gong, Xuehai Ju, Weihua Zhu, “Crystal density predictions for nitramines based on quantum chemistry”, Journal of Hazardous Materials, Vol. 141, No. 1, pp. 280-288, https://doi.org/10.1016/j.jhazmat.2006.06.135, 2007.
(JiaoXiongZhang 2018) F. Jiao, Y. Xiong, H. Li and C. Zhang, “Alleviating the energy & safety contradiction to construct new low sensitive and high energetic materials through crystal engineering”, CrystEngComm, Vol. 20, No. 18, pp. 1757-1768, 2018.
(QiuXiaoGongJu 2006) Ling Qiu, Heming Xiao, Xuedong Gong, Xuehai Ju, and Weihua Zhu, “Theoretical Studies on the Structures, Thermodynamic Properties, Detonation Properties, and Pyrolysis Mechanisms of Spiro Nitramines”, Journal of Physical Chemistry, Vol. 110, pp. 3797-3807, https://doi.org/10.1021/jp054169g, 2006.
(YangYanWangZhang 2014) Junqing Yang, Hua Yan, Guixiang Wang, Xueli Zhang, Tianyi Wang and Xuedong Gong, “Computational investigations into the substituent effects of N3, NF2, NO2, and NH2 on the structure, sensitivity and detonation properties of N, N′-azobis(1, 2, 4-triazole)”, Journal of Molecular Modeling, Vol. 20, No. 2148, https://doi.org/10.1007/s00897-014-2148-1, 2014.
(RettigRitterHelmer 2015) R. Rettig, N.C. Ritter, H. E. Helmer, S. Neumeier and R. F. Singer, “Single-Crystal Nickel-Based Superalloys Developed by Numerical Multi-Criteria Optimization Techniques: Design Based on Thermodynamic Calculations and Experimental Validation”, Modelling and Simulation in Materials Science and Engineering Vol. 23, https://doi.org/10.1088/0965-0393/23/3/035004, 2015.
(CouzinieSenkovMiracle 2018) J. P. Couzinie, O. N. Senkov, D. B. Miracle and G. Dirras, “Comprehensive data compilation on the mechanical properties of refractory high-entropy alloys,” ScienceDirect, Data in Brief, vol. 21, pp. 1622-1641, 2018.
(ConduitJones 2017) B. D. Conduit, N. G. Jones, H. J. Stone and G. J. Conduit, “Design of a Nickel-Base Superalloy Using a Neural Network”, Materials & Design, Vol. 131, pp. 358-365, 2017.
(Caron 2000) P. Caron, “Superalloys 2000,” in Ed. T. M. Pollock et al (TMS) pp 737-46, Seven Springs Pa., 2000.
(ChauvetKontisJagle 2018) E. Chauvet, P. Kontis, E. A. Jagle, B. Gault, D. Raabe, C. Tassin, J.-J. Blandin, R. Dendievel, B. Vayre, S. Abed and G. Martin, “Hot cracking mechanism affecting a non-weldable Ni-based superalloy produced by selective electron beam melting”, Acta Materialia, vol. 142, pp. 82-94, 2018.
(HarrisonToddMumtaz 2015) N. J. Harrison, I. Todd and K. Mumtaz, “Reduction of micro-cracking in nickel superalloys processed by Selective Laser Melting: A fundamental alloy design approach”, Acta Materialia, vol. 94, pp. 59-68, 2015.
(CarterAttallahReed 2012) L. N. Carter, M. M. Attallah, R. C. Reed, “Laser powder bed fabrication of Nickel-base superalloys: Influence of parameters; characterization, quantification and mitigation of cracking, Superalloys 2012, 12^{th} International Symposium on Superalloys, 2012.
(UCambridgeScheilEq 2021) University of Cambridge, “The Scheil Equation”, https://www.doitpoms.ac.uk/tlplib/solidification_alloys/scheil.php, Accessed on Sep. 12, 2021.
(ChrifiAlaouiNassik 2018) F. Z. Chrifi-Alaoui, M. Nassik, H. Azza and N. Selhaoui, “Enthalpies of formation of the compounds of ternary phases equilibria in the Al—Ni—Ti system at 1073 K”, Journal of Materials and Environmental Sciences, vol. 9, No. 4, pp. 1098-1109, 2018.
(RitterBozzelli 1991) E. R. Ritter, J. W. Bozzelli, “THERM: Thermodynamic property estimation for gas phase radicals and molecules”, International Journal of Chemical Kinetics, Vol. 23, No. 9, pp. 767-778, https://doi.org/10.1002/kin.550230903, 1991.
(MullerMichel 1995) C. Muller, V. Michel, G. Scacchi and G. M. Come, “THERGAS: a computer program for the evaluation of thermochemical data of molecules and free radicals in the gas phase”, J. Chem. Phys., Vol. 92, pp. 1154-1178, https://doi.org/10.1051/jcp/1995921154, 1995.
(QiLiLiWang 2011) Cai Qi, Sheng-Hua Li, Yu-Chuan Li, Yuan Wang, Xu-Kun Chen and Si-Ping Pang, “A novel stable high-nitrogen energetic material: 4,4′-azobis(1,2,4-triazole)”, Journal of Materials Chemistry, Vol. 21, No. 9, pp. 3221-3225, 2011.
(RicePaiHare 1999) Betsy M. Rice, Sharmila V. Pai, and Jennifer Hare, “Predicting heats of formation of energetic materials using quantum mechanical calculations”, Combustion and Flame, Vol. 118, No. 3, pp. 445-458, https://doi.org/10.1016/S0010-2180(99)00008-5, 1999.
(TalawarSivabalan 2009) M. B. Talawar, R. Sivabalan, T. Mukundan, H. Muthuraj an, A. K. Sikder, B. R. Gandhe, A. Subhananda Rao, Environmentally compatible next generation green energetic materials (GEMs), Journal of Hazardous Materials, Vol. 161, No. 2-3, pp. 589-607, https://doi.org/10.1016/j.jhazmat.2008.04.011, 2009.
(XiaoXuQiu 2008) H. M. Xiao, X. J. Xu and L. Qiu, “Theoretical design for high energetic materials”. Science Press, Beijing, 2008.
(KamletJacobs 1968) Mortimer J. Kamlet and S. J. Jacobs, “Chemistry of Detonations. I. A Simple Method for Calculating Detonation Properties of CHNO Explosives”, The Journal of Chemical Physics, Vol. 48, No. 23, pp. 23-35, https://doi.org/10.1063/1.1667908, 1968.
(PolitzerMartinezMurray 2009) Peter Politzer, Jorge Martinez, Jane S. Murray, Monica C. Concha and Alejandro Toro-Labb., “An Electrostatic Interaction Correction for Improved Crystal Density Prediction”, Molecular Physics Vol. 107, No. 19, pp. 2095-2101, https://doi.org/10.1080/00268970903156306, 2009.
(BulatToro-Labbe 2010) F. A. Bulat, A. Toro-Labb, T. Brinck, J. S. Murray and P. Politzer, “Quantitative analysis of molecular surfaces: areas, volumes, electrostatic potentials and average local ionization energies”, Journal of Molecular Modeling, Vol. 16, pp. 1679-1691, https://doi.org/10.1007/s00897-010-0692-x, 2010.
(KamletAdolph 1979) M. J. Kamlet, H. G. Adolph, “The relationship of Impact Sensitivity with Structure of Organic High Explosives. II. Polynitroaromatic explosives”, Propellants, Explosives, Pyrotechnics, Vol. 4, No. 2, pp. 30-34, https://doi.org/0.1002/prep.19790040204, 1979.
(QiuXiaoGongJu 2007) Ling Qiu, Heming Xiao, Xuedong Gong, Xuehai Ju, Weihua Zhu, “Crystal density predictions for nitramines based on quantum chemistry”, Journal of Hazardous Materials, Vol. 141, No. 1, pp. 280-288, https://doi.org/10.1016/j.jhazmat.2006.06,135, 2007.
(GuiXiangChunHong 2009) Wang Gui-xiang, Shi Chun-hong, Gong Xue-dong, and Xiao He-ming, “Theoretical Investigation on Structures, Densities, Detonation Properties, and the Pyrolysis Mechanism of the Derivatives of HNS”, The Journal of Physical Chemistry A, Vol. 113, No. 7, pp. 1318-1326, https://doi.org/10.1021/jp804950z, 2009.
(ZhangYun 1989) X. H. Zhang, Z. H. Yun, Explosive Chemistry; National Defense Industry Press: Beijing, 1989.
(HuangLiTanWen 2021) Xiaona Huang, Chongyang Li, Kaiyuan Tan, Yushi Wen, Feng Guo, Ming Li, Yongli Huang, Chang Q. Sun, Michael Gozin, Lei Zhang, “Applying Machine Learning to Balance Performance and Stability of High Energy Density Materials”, i Science, Vol. 24, No. 3, https://doi.org/10.1016/j.isci.2021.102240, 2021.
(JerrySeitzman 2021) Jerry M. Seitzman, “Rocket Propulsion Reacting Flow Issues”, Lecture Notes from AE6450 Rocket Propulsion, http://seitzman.gatech.edu/classes/ae6450/thermochemistry.pdf, Accessed on May 16, 2021.
(jpAgrawal 1998) J. P. Agrawal, “Recent trends in high-energy materials”, Progress in Energy and Combustion Science, vol. 24, no. 1, pp. 1-30, 1998.
(AgrawalSurveSonawane 2000) J. P. Agrawal, R. N. Surve, Mehilal, and S. H. Sonawane, “Some aromatic nitrate esters: synthesis, structural aspects, thermal and explosive properties”, Journal of Hazardous Materials, vol. 77, no. 1, pp. 11-31, 2000.
(BadgujarTalawarAsthana 2008) D. M. Badgujar, M. B. Talawar, S. N. Asthana, and P. P. Mahulikar, “Advances in science and technology of modern energetic materials: An overview”, Journal of Hazardous Materials, vol. 151, no. 2, pp. 289-305, 2008.
(BauerBenzKlapotke 2021) L. Bauer, M. Benz, T. M. Klapotke, T. Lenz, and J. Stierstorfer, “Polyazido-methyl Derivatives of Prominent Oxadiazole and Isoxazole Scaffolds: Synthesis, Explosive Properties, and Evaluation”, The Journal of Organic Chemistry, vol. 86, no. 9, pp. 6371-6380, 2021.
(DuQuWangCuiWang 2021) Y. Du, Z. Qu, H. Wang, H. Cui, and X. Wang, “Review on the Synthesis and Performance for 1,3,4-Oxadiazole-Based Energetic Materials”, Propellants, Explosives, Pyrotechnics, vol. 46, no. 6, pp. 860-874, 2021.
(EatonGilardiZhang 2000) P. E. Eaton, R. L. Gilardi, and M. X. Zhang, “Polynitrocubanes: Advanced High-Density, High-Energy Materials”, Advanced Materials, vol. 12, no. 15, pp. 1143-1148, 2000.
(FanJu 2008) X. W. Fan and X. H. Ju, “Theoretical studies on four-membered ring compounds with NF2, ONO2, N3, and NO2 groups”, Journal of Computational Chemistry, vol. 29, no. 4, pp. 505-513, 2008.
(FershtatMakhova 2020) L. L. Fershtat and N. N. Makhova, “1,2,5-Oxadiazole-Based High-Energy-Density Materials: Synthesis and Performance”, ChemPlusChem, vol. 85, no. 1, pp. 13-42, 2020.
(GaoLiuSuZhangYang 2014) W. Gao, X. Liu, Z. Su, S. Zhang, Q. Yang, Q. Wei, S. Chen, G. Xie, X. Yang, and S. Gao, “High-energy-density materials with remarkable thermostability and insensitivity: syntheses, structures and physicochemical properties of Pb(ii) compounds with 3-(tetrazol-5-yl) triazole”, Journal of Materials Chemistry A, vol. 2, no. 30, pp. 11958-11965, 2014.
(GeithKlapotkeWeigand 2004) J. Geith, T. M. Klapotke, J. Weigand, and G. Holl, “Calculation of the Detonation Velocities and Detonation Pressures of Dinitrobiuret (DNB) and Diaminotetrazolium Nitrate (HDAT-NO3)”, Propellants, Explosives, Pyrotechnics, vol. 29, no. 1, pp. 3-8, 2004.
(GutowskiRogersDixon 2007) K. E. Gutowski, R. D. Rogers, and D. A. Dixon, “Accurate Thermochemical Properties for Energetic Materials Applications. II. Heats of Formation of Imidazolium-, 1,2,4-Triazolium-, and Tetrazolium-Based Energetic Salts from Isodesmic and Lattice Energy Calculations”, The Journal of Physical Chemistry B, vol. 111, no. 18, pp. 4788-4800, 2007.
(HaoLiuZhaiQiuMa 2020) L. Hao, X. Liu, D. Zhai, L. Qiu, C. Ma, P. Ma, and J. Jiang, Theoretical Studies on the Performance of HMX with Different Energetic Groups, ACS Omega, vol. 5, no. 46, pp. 29922-29934, 2020.
(HenryParkinsonMayer 2001) D. J. Henry, C. J. Parkinson, P. M. Mayer, and L. Radom, “Bond Dissociation Energies and Radical Stabilization Energies Associated with Substituted Methyl Radicals”, The Journal of Physical Chemistry A, vol. 105, no. 27, pp. 6750-6756, 2001.
(HuangTangImlerParrish 2020) W. Huang, Y. Tang, G. H. Imler, D. A. Parrish, and J. n. M. Shreeve, “Nitrogen-Rich Tetrazolo[1,5-b]pyridazine: Promising Building Block for Advanced Energetic Materials”, Journal of the American Chemical Society, vol. 142, no. 7, pp. 3652-3657, 2020.
(Koch 2015) E. C. “Koch, TEX 4,10-Dinitro-2,6,8,12-tetraoxa-4,10-diazatetracyclo[5.5.0.05,9.03,11]-dodecane Review of a Promising High Density Insensitive Energetic Material”, Propellants, Explosives, Pyrotechnics, vol. 40, no. 3, pp. 374-387, 2015.
(LiGuoLiSong 2009) M. Li, X. Guo, F. Li, and H. Song, “Theoretical Studies on the Structures, Thermodynamic Properties, Detonation Performance, and Pyrolysis Mechanisms for Six Dinitrate Esters”, Chinese Journal of Chemistry, vol. 27, no. 10, pp. 1871-1878, 2009.
(LiJuZhang 2017) X. H. Li, W. W. Ju, and R. Z. Zhang, “Theoretical studies on nitrogen-rich furoxan-based heterocyclic derivatives”, Journal of Physical Organic Chemistry, vol. 30, no. 5, e3627, 2017.
(LiaoJuZhaoYi 2010) X. Liao, X. H. Ju, F. Q. Zhao, and J. H. Yi, “Substituent Effects on Thermodynamic and Detonation Properties of Polynitrobenzenes”, Propellants, Explosives, Pyrotechnics, vol. 35, no. 6, pp. 567-571, 2010.
(LiuWangWangGong 2013) H. Liu, F. Wang, G.-X. Wang, and X.-D. Gong, “Theoretical studies on 2-(5-amino-3-nitro-1,2,4-triazolyl)-3,5-dinitropyridine (PRAN) and its derivatives”, Journal of Physical Organic Chemistry, vol. 26, no. 1, pp. 30-36, 2013.
(McDonaldBennionLeone 2016) K. A. McDonald, J. C. Bennion, A. K. Leone, and A. J. Matzger, “Rendering non-energetic microporous coordination polymers explosive”, Chemical Communications, vol. 52, no. 72, pp. 10862-10865, 2016.
(McDonaldSethMatzger 2015) K. A. McDonald, S. Seth, and A. J. Matzger, “Coordination Polymers with High Energy Density: An Emerging Class of Explosives”, Crystal Growth & Design, vol. 15, no. 12, pp. 5963-5972, 2015.
(NairAsthanaRaoGandbe 2010) U. R. Nair, S. N. Asthana, A. S. Rao, and B. R. Gandhe, “Advances in High Energy Materials (Review Paper)”, Defence Science Journal, vol. 60, no. 2, pp. 137, 2010.
(QiuXiaoGongJuZhu 2006) L. Qiu, H. Xiao, X. Gong, X. Ju, and W. Zhu, “Theoretical Studies on the Structures, Thermodynamic Properties, Detonation Properties, and Pyrolysis Mechanisms of Spiro Nitramines”, The Journal of Physical Chemistry A, vol. 110, no. 10, pp. 3797-3807, 2006.
(QiuXiaoZhuXiaoGong 2006) L. Qiu, H.-M. Xiao, W.-H. Zhu, X.-H. Xiao, and X.-D. Gong, “Theoretical Study on the High Energy Density Compound Hexanitrohexaazatricyclotetradecanedifuroxan”, Chinese Journal of Chemistry, vol. 24, no. 11, pp. 1538-1546, 2006.
(RaviGoreTewariSikder 2012) P. Ravi, G. M. Gore, S. P. Tewari, and A. K. Sikder, “Quantum Chemical Studies on the Structure and Performance Properties of 1,3,4,5-Tetranitropyrazole: A Stable New High Energy Density Molecule”, Propellants, Explosives, Pyrotechnics, vol. 37, no. 1, pp. 52-58, 2012.
(SethMatzger 2017) S. Seth and A. J. Matzger, “Coordination Polymerization of 5,5′-Dinitro-2H,2H′-3,3′-bi-1,2,4-triazole Leads to a Dense Explosive with High Thermal Stability”, Inorganic Chemistry, vol. 56, no. 1, pp. 561-565, 2017.
(TalawarSivabalanMukundan 2009) M. B. Talawar, R. Sivabalan, T. Mukundan, H. Muthurajan, A. K. Sikder, B. R. Gandhe, and A. S. Rao, “Environmentally compatible next generation green energetic materials (GEMs)”, Journal of Hazardous Materials, vol. 161, no. 2, pp. 589-607, 2009.
(TanLuLiuYan 2021) L. Tan, X. Lu, N. Liu, and Q.-L. Yan, “Further enhancing thermal stability of thermostable energetic derivatives of dibenzotetraazapentene by polydopamine/graphene oxide coating”, Applied Surface Science, vol. 543, 148825, 2021.
(TangKumarShreeve 2017) Y. Tang, D. Kumar, and J. n. M. Shreeve, “Balancing Excellent Performance and High Thermal Stability in a Dinitropyrazole Fused 1,2,3,4-Tetrazine”, Journal of the American Chemical Society, vol. 139, no. 39, pp. 13684-13687, 2017.
(TappanBrill 2003) B. C. Tappan and T. B. Brill, “Thermal Decomposition of Energetic Materials 86. Cryogel Synthesis of Nanocrystalline CL-20 Coated with Cured Nitrocellulose”, Propellants, Explosives, Pyrotechnics: An International Journal Dealing with Scientific and Technological Aspects of Energetic Materials, vol. 28, no. 5, pp. 223-230, 2003.
(ThottempudiGaoShreeve 2011) V. Thottempudi, H. Gao, and J. n. M. Shreeve, Trinitromethyl-Substituted 5-Nitro- or 3-Azo-1,2,4-triazoles: Synthesis, Characterization, and Energetic Properties, Journal of the American Chemical Society, vol. 133, no. 16, pp. 6464-6471, 2011.
(ThottempudiShreeve 2011) V. Thottempudi and J. n. M. Shreeve, “Synthesis and Promising Properties of a New Family of High-Density Energetic Salts of 5-Nitro-3-trinitromethyl-1H-1,2,4-triazole and 5,5 ‘-Bis(trinitromethyl)-3,3’-azo-1H-1,2,4-triazole”, Journal of the American Chemical Society, vol. 133, no. 49, pp. 19982-19992, 2011.
(WangGongLiuXiao 2010) G. Wang, X. Gong, Y. Liu, and H. Xiao, “A theoretical investigation on the structures, densities, detonation properties, and pyrolysis mechanism of the nitro derivatives of phenols”, International Journal of Quantum Chemistry, vol. 110, no. 9, pp. 1691-1701, 2010.
(WangGongXiao 2009) G. X. Wang, X. D. Gong, and H. M. Xiao, Theoretical investigation on density, detonation properties, and pyrolysis mechanism of nitro derivatives of benzene and aminobenzenes, International Journal of Quantum Chemistry, vol. 109, no. 7, pp. 1522-1530, 2009.
(WuLiHuZhangZhu 2020a) Q. Wu, M. Li, Q. Hu, Z. Zhang, and W. Zhu, “Effects of boron doping on structural, electronic, elastic, and optical properties of energetic crystal 2,6-diamino-3,5-dinitropyrazine-1-oxide: a theoretical study using the first principles calculation and Hirshfeld surface analysis”, Journal of Molecular Modeling, vol. 26, no. 3, 41, 2020.
(WuLiHuZhangZhu 2020b) Q. Wu, M. Li, Q. Hu, Z. Zhang, and W. Zhu, “First-principle study and Hirshfeld surface analysis on the effect of H2O, NH3 and H2S on structural, electronic, elastic, optical and thermodynamic properties of a novel high-energy crystal 2,4,6-triamino-5-nitropyrimidine-1,3-dioxide”, Journal of Materials Science, vol. 55, no. 1, pp. 237-249, 2020.
(YangWangGongZhangWang 2018) J. Yang, G. Wang, X. Gong, J. Zhang, and Y. A. Wang, “High-Energy Nitramine Explosives: A Design Strategy from Linear to Cyclic to Caged Molecules”, ACS Omega, vol. 3, no. 8, pp. 9739-9745, 2018.
(YinShreeve 2015) P. Yin and J. n. M. Shreeve, “From N-Nitro to N-Nitroamino: Preparation of High-Performance Energetic Materials by Introducing Nitrogen-Containing Ions”, Angewandte Chemie International Edition, vol. 54, no. 48, pp. 14513-14517, 2015.
(YongjinShuhong 2019) C. Yongjin and B. Shuhong, “High Energy Density Material (HEDM)-Progress in Research Azine Energetic Compounds”, Johnson Matthey Technology Review, vol. 63, no. 1, pp. 51-72, 2019.
(YuYinZhangHeImler 2017) Q. Yu, P. Yin, J. Zhang, C. He, G. H. Imler, D. A. Parrish, and J. n. M. Shreeve, “Pushing the Limits of Oxygen Balance in 1,3,4-Oxadiazoles”, Journal of the American Chemical Society, vol. 139, no. 26, pp. 8816-8819, 2017.
(YuanGanJiangZhu 2021) W. S. Yuan, Y. D. Gan, C. L. Jiang, S. H. Zhu, M. J. Zhang, F. S. Liu, B. Tang, D. Hong, and Q. J. Liu, “First-principles calculations of the electronic, vibrational, and thermodynamic properties of 2,6-diamino-3,5-dinitropyrazine-1-oxide (LLM-105)”, Chemical Physics, vol. 548, 111232, 2021.
(ZhangShreeve 2014) J. Zhang and J. n. M. Shreeve, “3,3′-Dinitroamino-4,4′-azoxyfurazan and Its Derivatives: An Assembly of Diverse NO Building Blocks for High-Performance Energetic Materials”, Journal of the American Chemical Society, vol. 136, no. 11, pp. 4437-4445, 2014.
(ZhangDuWangGongHuang 2011) J. Y. Zhang, H. C. Du, F. Wang, X. D. Gong, and Y. S. Huang, “DFT Studies on a High Energy Density Cage Compound 4-Trinitroethyl-2,6,8,10,12-pentanitrohezaazaisowurtzitane”, The Journal of Physical Chemistry A, vol. 115, no. 24, pp. 6617-6621, 2011.
(ZhangZhangDengQi 2017) W. Zhang, J. Zhang, M. Deng, X. Qi, F. Nie, and Q. Zhang, “A promising high-energy-density material”, Nature Communications, vol. 8, no. 1, 181, 2017.
(ZhangZhuXiao 2010) X. Zhang, W. Zhu, and H. Xiao, “Theoretical studies on heats of formation, detonation properties, and bond dissociation energies of monofurazan derivatives”, International Journal of Quantum Chemistry, vol. 110, no. 8, pp. 1549-1558, 2010.
(ZhangWangJiZhouZhou 2008) Z. Z. Zhang, B. Z. Wang, Y. P. Ji, Y. S. Zhou, C. Zhou, Q. Liu, and C. H. Zhu, “Study progress of several high energy density materials (HEDM)”, Chinese Journal of Explosives & Propellants 2, 2008.
(ZhaoLu 2012a) G. Zhao and M. Lu, “Theoretical investigation on the structures, densities, and detonation properties of polynitrotetraazaoctahydroanthracenes”, International Journal of Quantum Chemistry, vol. 112, no. 16, pp. 2794-2800, 2012.
(ZhaoLu 2013a) G. Zhao and M. Lu, “A Theoretical investigation of a potential high energy density compound 3, 6, 7, 8-tetranitro-3, 6, 7, 8-tetraaza-tricyclo[3.1. 1.1 (2, 4)] octane”, Quimica Nova, vol. 36, pp. 513-518, 2013.
(ZhaoLu 2013b) G. Zhao and M. Lu, “Theoretical investigations of pyridine derivatives as potential high energy density materials”, Journal of Physical Organic Chemistry, vol. 26, no. 3, pp. 211-217, 2013.
(Vaghjiani 2021) Ghanshyam L Vaghjiani, “Private Communication”, Mar. 31, 2021.
(Kaewchoothong 2017) N. Kaewchoothong, K. Maliwan, C. Nuntadusit, “Numerical simulations on low and heat transfer in ribbed two-pass square channels under rotational e ects”. In IOP Conference Series: Materials Science and Engineering, Proceedings of the Computational Fluid Dynamics in Research and Industry (CFDRI 2017), Songhkla, Thailand, 3-4 Aug. 2017; IOP Publishing: Bristol, U K, 2017; p. 012004.
(Brunetaud 1982) R. Brunetaud, D. Coutsouradis, T. B. Gibbons, Y. Lindblom, D. B. Meadowcroft and R. Stickler, “High Temperature Alloys for Gas Turbines 1982”, Proceedings of a Conference held in Liege, Belgium, Oct. 4-6, 1982.
(AngelBasak 2020) N. M. Angel and A. Basak, “Review on the fabrication of metallic single crystal turbine blades with a commentary on repair via additive manufacturing”, Journal of Manufacturing and Materials Processing, vol. 4, no. 101, 2020.
(FengZhangGaoPei 2021) R. Feng, C. Zhang, M. C. Gao, Z. Pei, F. Zhang, Y. Chen, D. Ma, K. An, J. D. Poplawsky, L. Ouyang, Y. Ren, J. A. Hawk, M. Widom and P. K. Liaw, “High-throughput design of high-performance lightweight high-entropy alloys”, Nature Communications, Vol. 12, No. 2021, pp. 4329, 2021.
(BorgFreyMohPollock 2020) C. K. H. Borg, C. Frey, J. Moh, T. M. Pollock, S. Gorsse, D. B. Miracle, O. N. Senkov, B. Meredig and J. E. Saal, “Expanded dataset of mechanical properties and observed phases of multi-principal element alloys”, Scientific Data 7, Article 420, 2020.
(ZhuNguyenNgAn 2018) Z. G. Zhu, Q. B. Nguyen, F. L. Ng, X. H. An, X. Z. Liao, P. K. Liaw, S. M. L. Nai, J. Wei, Hierarchical microstructure and strengthening mechanisms of a CoCrFeNiMn high entropy alloy additively manufactured by selective laser melting, Scripta Materialia, Volume 154, Pages 20-24, 2018.
(WilsonHeidNovakBeese 2019) A. E. Wilson-Heid, T. C. Novak and A. M. Beese, “Characterization of the Effects of Internal Pores on Tensile Properties of Additively Manufactured Austenitic Stainless Steel 316L”, Experimental Mechanics, Vol. 59, pp. 793-804, 2019.
(FuHaberlandKlapdorRule 2017) W. Fu, C. Haberland, E. V. Klapdor, D. Rule and S. Piegert, “Streamlined Frameworks for Advancing Metal Based Additive Manufacturing Technologies in Gas Turbine Industry”, Proceedings of the 1^{st} Global Power and Propulsion Forum, GPPF 2017, GPPF-2017-182, Zurich, Switzerland, www.pps.global, January 16-18, 2017.
(GangulyDatta 2007) S. Ganguly, S. Datta, and N. Chakraborti, “Genetic Algorithms in Optimization of Strength and Ductility of Low-Carbon Steels,” Materials and Manufacturing Processes, vol. 22, no. 5, pp. 650-658, 2007.
(NIST 2019) NIST, “AI/ML, for Data Extraction and Uncertainty Predictions,” [Online]. Available: https://www.nist.gov/programs-projects-aiml-data-extraction-and-uncertainty-predictions, accessed on Aug. 20, 2019.
(NewellHagerty 2020) S. A. Newell, J. M. Hagerty, J. P. Pfeifenberger, B. Zhou, E. Shorin, P. Fitz, “Baseline energy usage from PJM Measured H-class and F-class heat rate performance,” Available: https://www.pjm/com8˜/media/committees-groups/committees/mic/20180425-special/20180425-pjm-2018-cost-of-new-entry-study.ashx, Accessed on Sep. 17, 2020.
(FederalAviation 2020) Federal Aviation Administration, “FAA Aerospace Forecast Fiscal Years 2019-2039,” U. S Fleet Growth Data per Oliver Wyman Associates, Available: https://www.faa.gov/data_research/aviation/aerospace_forecasts/media/FY2019-39_FAA_Aerospace_Forecase.pdf, Accessed on Sep. 17, 2020.
(T&DworldSiemens 2021) T&D World, “Siemens Successfully Tests 3D-Printed Gas Turbine Blades,” Available: https://www.tdworld.com/renewables/article/20969284/siemens-successfully-tests-3dprinted-gas-turbine-blades/, Accessed on Aug. 14, 2021.
(BureauTransportation 2020) Bureau of Transportation Statistics, “Available Seat-miles (the number of seats and the distance flown in thousands (000)),” Available: https://www.transtats.bts.gov/Data_Elements.aspx?Data=4, Accessed on Sep. 17, 2020.
(DE-FOA-0002337) Department of Energy, ARPA-E Archived Funding Opportunities, “DE-FOA-0002337: ULTRAHIGH TEMPERATURE IMPERVIOUS MATERIALS ADVANCING TURBINE EFFICIENCY (ULTIMATE)”, https://arpa-e-foa.energy.gov/Default.aspx?foaId=8c403d18-ded6-48d0-be56-a5ab5922c121, Accessed on Jul. 24, 2021.
(DE-FOA-0002553) Department of Energy, “Office of Energy Efficiency and Renewable Energy (EERE) FY21 Advanced Manufacturing Office Multi-Topic FOA”, https://eere-exchange.energy.gov/, Accessed on Aug. 8, 2021.
(NasaVision2040 2021) NASA Vision 2040: A Roadmap for Integrated, Multiscale Modeling and Simulation of Materials and Systems, NASA/CR-2018-219771, https://ntrs. nasa.gov/archive/nasa/casi.ntrs.nasa.gov/20180002010.pdf, Accessed on Aug. 7, 2021.
(Kulkarni 2021) Anand A. Kulkarni, “E-mail Communication”, Aug. 2, 2021.
(NavySbir211085 2021) Navy SBIR 21.1 Topic N211-085, “Developing Alloy Compositions Conducive to Additive Manufacturing”, https://www.navysbir.com/n21_1/N211-0.85.htm, Accessed on Aug. 8, 2021.

### SUMMARY OF THE INVENTION

This invention presents a generic framework for innovative deployment of AI, ML and/or DL approaches, for purpose of accelerating the discovery or design of new energetic materials (materials with high energy density), via accurate assessments of their physico-chemical properties from available databases. The invention identifies the specific molecular properties impacting the energy density of energetic materials, propellants in particular.

### 1. Framework for a Generic Engine for Predicting Properties of Energetic Materials, One Capable of Interfacing with, and Receiving Support from, Advanced Physics-Based Prediction Models

The invention presents method and apparatus capable of interfacing with, and receiving benefits from, physics-based models, i.e., models that account for physical dependencies, and factor in the underlying physics as a priori information, during the prediction process, for the purpose of making the most of the usually limited input data available. With such hybrid prediction methods and apparatus, one may predict the thrust or the specific impulse, as highlighted in FIG. 11.

There can be great benefits derived from combining ML with physics-based modeling approaches for energetic materials, for improved prediction accuracy. Such modeling approaches can offer physical insight (a guiding reference, or a sanity check, derived from the underlying physics), as unexplored regions of the composition space are investigated. These approaches may include thermo-physical or thermo-chemical equilibrium calculations, molecular dynamic, first-principle calculations (quantum mechanics) or group additivity methods.

In case an ANN is deemed suitable for the application at hand, the invention can employ custom kernel functions consistent with the underlying physics, for the purpose of attaining tighter coupling, better prediction, and extracting the most out of the—usually limited—input data available.

### 2. Framework for a Generic Engine for Predicting Properties of Energetic Materials, One Capable of Supporting Joint Optimization

The prediction engine epitomizes our innovative framework for the application of machine learning to the development of energetic materials with desired properties. During design of energetic materials, like propellants, designers often need to strike trade-offs between material properties. For example, designers may look to optimize a propellant for high energy density or for high stability. There is a trade-off between detonation performance and thermal stability, because a high detonation performance of HEDMs relies on the large energy difference between the reactants and the reaction products, whereas the high stability of HEDMs requires a sufficiently high energy barrier to prevent the uncontrolled initiation of such reactions. A highly stable propellant is good for energy storage and transport. There also may be a trade-off between the energy- and the mass-density of propellants.

The scheme presented for joint optimization of the properties of energetic materials is an extension of similar approaches originally developed for the joint optimization of alloy properties (SteingrimssonFanKulkarni 2020), (SteingrimssonFanKulkarni 2021).

### 3. Framework for a Generic Engine for Predicting Properties of Energetic Materials, One Capable of Accounting for Properties Both at Macro-Level and Micro-Level (Molecular Level)

We refer to macro-level optimization, listed in FIG. 12, as optimization that does not explicitly make use during the optimization process of information related to molecular structure, such as the bond lengths, of the propellant candidates under consideration. Micro-level optimization, also listed in FIG. 12, does, however, explicitly make use of information related to the molecular structure, during the optimization process.

Conceptually, one wants to be able to pick a molecule, feed into the framework and identify whether this is a likely candidate for a high-energy density material or not (rather than having to try empirically). Conclusive experimental verification on test molecules might take long time (years). Instead, we want to be able to pick out the behavior of the candidate molecules, and based on the data, tell how specific ingredients, such as nitrides or carbons, can affect this behavior. This helps inform the assessment of whether a candidate molecule is likely to be a good HDEM or not.

### 4. Framework for a Generic Engine for Predicting Properties of Energetic Materials, One Capable of Multi-Linear Regression of Descriptors, Supporting Associated Properties and Offering Physics-Based Interpretation (of Employing Simple Prediction Models when Appropriate)

As opposed to applying machine learning, narrowly defined in terms of regression techniques, neural networks (single-layer or multi-layer), Bayesian graphical models, support vector machines or decision trees, to the identification of energetic materials of interest, we reformulate the task in the broader context of engineering optimization. We recommend picking an optimization technique suitable for the application at hand and the data available. But we certainly include ML techniques in that consideration.

### 5. Framework for a Generic Engine for Predicting Properties of Energetic Materials, One Capable of Accurately Encoding Properties and Behavior of Complex Molecular Structures into Descriptors

How to craft a set of parameters (descriptors), that (a) accurately capture the properties and behavior of complex molecular structures, (b) can be imported into an ML algorithm for analysis, may be considered far from obvious. Our formulation of descriptors capturing the structure of complex molecules, including derivatives, substituent and functional groups, draws upon similar formulations from (SteingrimssonFanKulkarni 2020) and (SteingrimssonFanKulkarni 2021). Ref. (SteingrimssonFanKulkarni 2020) and (SteingrimssonFanKulkarni 2021) also describe a structurally similar approach for encoding the parameters of processes for heat treatment of HEAs into descriptors for ML analysis. The approach for encoding the parameters of the molecular structures exhibits structural similarity with the approach outlined in (SteingrimssonFanKulkarni 2020) and (SteingrimssonFanKulkarni 2021) for parametrizing the microstructure of alloys.

### DESCRIPTION OF THE DRAWINGS

FIG. 1 presents a high-level overview of the key categories of energetic materials. Double base solid propellants consist of nitrocellulose and nitroglycerine, plus additives in a small quantity. There is no separate fuel and oxidizer. In composite solid propellants, separate fuel and oxidized chemicals are used. These are intimately mixed in solid grains. In processing solid propellants, fuel (a fluid of varying consistency) and an oxidizer (a powder) are usually blended carefully and poured into a rocket case as a viscous semisolid, whose viscosity properties tend to be favorable for handling. Solid propellants have the advantage of requiring minimum maintenance and providing instant readiness (for explosion leading to thrust). However, in terms of disadvantages, the solid propellants require carefully controlled storage conditions (in order to maintain consistent viscosity of the semisolid). Handling problems may also result, when solid propellants are being handled in very large volumes.

FIG. 2 expands on FIG. 1 and further breaks down the categories of liquid rocket propellants. Liquid monopropellants contain certain unstable, liquid chemicals which, under proper conditions, will decompose and release energy, as illustrated in FIG. 6. Monopropellants are usually used in smaller rockets, because the monopropellants tend to provide less power or energy than bipropellants. Most liquid chemical rockets use two separate propellants (a liquid bipropellant), i.e., a fuel and an oxidizer, as shown in FIG. 7. A hypergolic liquid bipropellant ignites spontaneously (within 75 msec) upon contact of the fuel with the oxidizer. A liquid tripropellant consists of a three-propellant system, where one of the components may be a solid. A tripropellant may consist of a solid fuel, liquid fuel and an oxidizer. An ionic liquid propellant usually refers to a salt that is in the liquid state.

FIG. 3 expands on FIG. 2 and further breaks down the categories of storable oxidizers.

FIG. 4 similarly expands on FIG. 2 and further breaks down the categories of monopropellants.

FIG. 5 presents the forces that act on a rocket during a coasting or a powered flight. FIG. 5a presents the forces that act on a rocket during coasting flight. FIG. 5b illustrates the forces acting on a rocket during powered flight. The drag force varies with the velocity squared. The gravity force varies with the remaining rocket mass. The thrust force varies during motor burn according to a thrust curve.

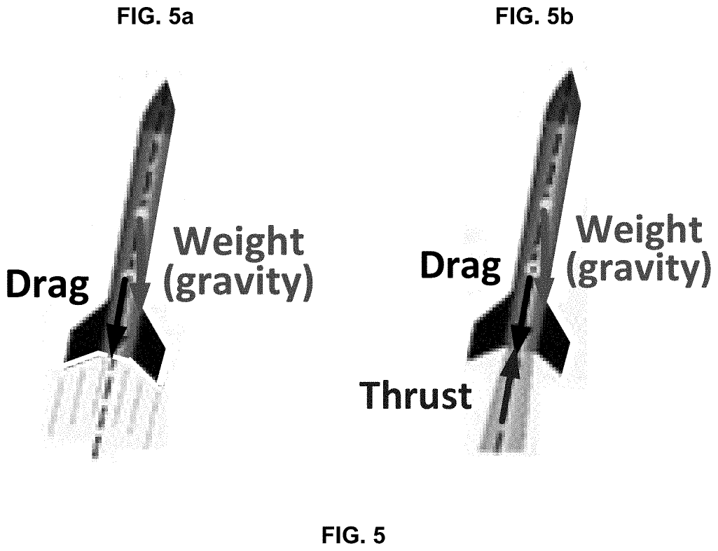

FIG. 6 offers an overview over the key principles behind the propulsion of a rocket fueled by a monopropellant.

FIG. 7 provides an overview over the key principles behind the propulsion of a rocket fueled by a bipropellant.

FIG. 8 provides a high-level introduction to sequential learning. Development of HEDMs has traditionally been slow, in part, due to the extremely high hazard involved, and the high cost of the experimental research involving HEDMs, as well as due to the long-term life cycle of their characterization, manufacturing, testing, and inspection (HuangLiTanWen 2021). FIG. 8a provides high-level comparison of sequential learning with traditional materials development. FIG. 8b shows data from new experiments (predictions) can be added to the training set and fed into the machine learning model.

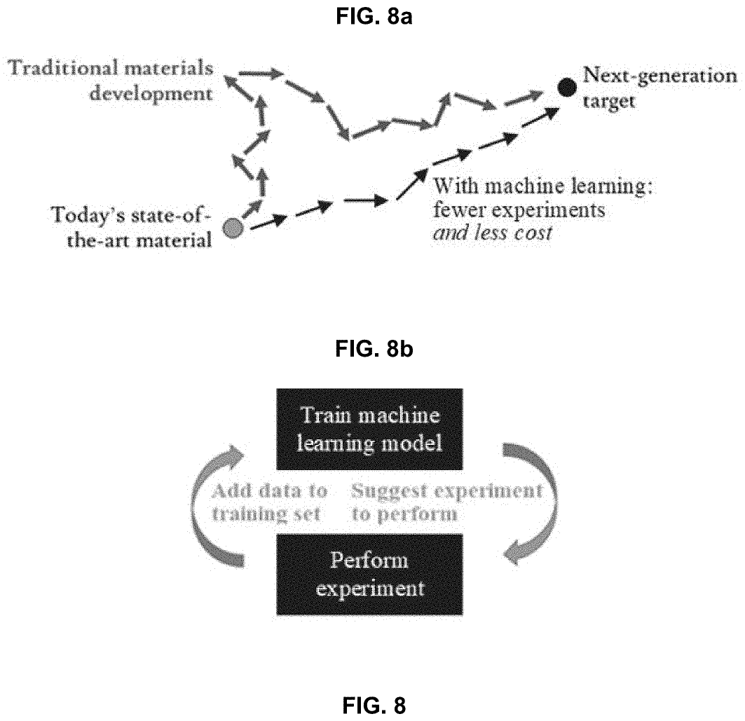

FIG. 9 summarizes the primary sources impacting the energy density of energetic materials.

FIG. 10 provides a more detailed overview of the energy density properties of energetic materials than shown in FIG. 9. FIG. 10 also presents a more detailed overview of the sources impacting the energy density and highlights some of the dependencies between the sources.

FIG. 11 illustrates a hybrid system consisting of a “standard” ML model and an analytical, physics-based model at a high level.

FIG. 12 summarizes an approach, where fuel properties are optimized at macro-scale, prior to optimization at micro-scale.

FIG. 13 presents a master architecture (a dependency diagram), one capturing the prediction engine.

FIG. 14 presents the relationship, per the dependency diagram in FIG. 13 between the caller, that initiates a request, and the callee, that responds to a call.

FIG. 15 captures the essential mechanism of Export, Transform and Load.

FIG. 16 shows the standard enthalpy change of formation in BornHaber diagram for lithium fluoride. FIG. 16 has been adapted from (WikipediaEnthalpyFormation 2021).

FIG. 17 presents five (5) derivative structures of diphenyl oxalate.

FIG. 18 provides high-level overview of molecular dynamics. FIG. 18a present high-level structure of a classical molecular dynamics simulator. FIG. 18b and FIG. 18c are is presented in context with molecular dynamics simulation of oxidation of an aluminum layer of an Fe—Ni—Co substrate, illustrating the formation of a pinhole. FIG. 18b presents an initial configuration, but FIG. 18c a final configuration. The atomic structures presented were adapted from (JonesZhouLammps 2021).

FIG. 19 shows subgroups within an 2,2,4-trimethylpentane (iso-octane) molecule that are relevant to Benson group-increment theory (group additivity). FIG. 19a shows the 2,2,4-trimethylpentane (iso-octane) molecule, but FIG. 19b lists the subgroups. The figure has been adapted from (HenryCurran 2019).

FIG. 20 highlights two cases of 1,5 pentane interaction within the 2,2,4-trimethylpentane (iso-octane) molecule. FIG. 20a illustrates the first case of 1,5 pentane interaction within the 2,2,4-trimethylpentane (iso-octane) molecule. FIG. 20b illustrates the second case of 1,5 pentane interaction within the 2,2,4-trimethylpentane (iso-octane) molecule.

FIG. 21 presents schematic Pareto frontiers capturing optimal trade-offs for two properties being jointly optimized. FIG. 21a presents a schematic Pareto frontier capturing optimal trade-offs between performance of an energetic material, measured in terms of the specific impulse, and thermal stability, measured in terms of the self-accelerating decomposition temperature. FIG. 21b presents a schematic Pareto frontier capturing optimal trade-offs between performance of an energetic material, measured in terms of the specific impulse, and its mass density.

FIG. 22 presents a schematic presentation of the neural network used in the first step of the Bayesian bootstrapping approach for joint optimization of properties of energetic materials (adapted from ConduitJones 2017).

FIG. 23 presents the following figures of merit: 25% blade cooling reduction, 0.6% increase in efficiency, and 3% increase in turbine power. FIG. 23a presents high-entropy alloys of type HEA-3 which exhibit superior yield strength at elevated temperature, compared to the superalloys Haynes 230, Hastelloy X and Inconel 600. FIG. 23b shows a turbine blade and highlights the desire for a 200° C. increase in the operational temperature of a turbine. FIG. 23c depicts a rotating gas turbine, and notes that combined cycle efficiency of a gas-turbine engine in excess of 65% can be achieved through such an increase in the combustor firing temperature.

FIG. 24 presents a representative diagram showing the cooling mechanisms in a turbine blade (Reproduced from (Kaewchoothong 2017) under the terms of the Creative Commons Attribution 3.0 License from https://creativecommons.org/licenses/by/3.0/).

FIG. 25 captures the anticipated disruption of HEAs for extreme operational environments, such as found in turbines.

FIG. 26 presents a system compatibility study for HEAs for turbine components (SteingrimssonFanKulkarni 2021).

FIG. 27 presents representative processes for alloy design. FIG. 27a presents a relatively simple process for alloy design. FIG. 27b presents a representative ICME process for the design of refractory HEAs suitable for extreme service environments, such as in a turbine. FIG. 27a was adapted from the work of Dr. Rui Feng, Dr. Michael Gao, Dr. Cheung Zhang, Prof. Lizhi Ouyang and Prof. Peter K. Liaw.

FIG. 28 presents a product concept for a plugin for joint optimization of mechanical properties of alloys at a high level.

FIG. 29 presents a diagram capturing a high-level approach to joint ML optimization of HEA mechanical and environmental properties. FIG. 29a lists the primary inputs to the joint optimization process (the composition) as well as the primary outputs (the mechanical and oxidation properties). FIG. 29b provides high-level depiction of the interface between the joint optimization and tools for calculating phase diagrams.

FIG. 30 captures a joint optimization problem formulated for the BCC composition Nb—Ta—Cr—V—Ti—Zr (SteingrimssonFanKulkarni 2020), (SteingrimssonFanKulkarni 2021).

FIG. 31 provides a general introduction to HEAs. FIG. 31a presents the design of HEAs as generalization of the design of Nickel-based superalloys, in context with the phase diagram for a Al—Ni—Ti system. The phase diagram for the Al—Ni—Ti system was adapted from FIG. 1 of (ChrifiAlaouiNassik 2018). FIG. 31b presents rational for element selection for the design of a Ni-based superalloy. Both FIG. 31a and FIG. 31b were adapted from the work of Dr. Rui Feng, Dr. Michael Gao, Dr. Cheung Zhang, Prof. Lizhi Ouyang and Prof. Peter K. Liaw.

FIG. 32 presents the primary input sources and observed outputs for hot cracking in a laser powder bed AM system.

FIG. 33 describes the process of solute redistribution during solidification of an alloy, according to the Scheil model. FIG. 33 was replicated from (WikipediaScheilEquation 2021).

FIG. 34 sheds light on how a plugin for machine learning applied to energetic materials can be integrated into established tools used for thermochemical analysis.

FIG. 35 captures preliminary results for the predicted vs. actual specific impulse for a simple prediction model relevant to energetic materials (propellants).

### DETAILED DESCRIPTION OF THE PREFERRED EMBODIMENTS

### 1. Definitions

Table 1 captures the primary acronyms used in the patent.

TABLE 1
Summary of the primary definitions and acronyms.

| Name | Definition |
|---|---|
| AI | Artificial Intelligence |
| AM | Additive Manufacturing |
| API | Application Program Interface |
| BDE | Bond Dissociation Energy |
| BGIT | Benson Group-Increment Theory |
| CALPHAD | CALculations of PHAse Diagrams |
| CCE | Combined Cycle Efficiency |
| CEA | Chemical Equilibrium with Applications |
| CHNO | Carbon Hydrogen Nitrogen and Oxygen |
| CPFEM | Crystal Plasticity Finite Element |
| DDD | Discrete Dislocation Dynamics |
| DFT | Density Functional Theory |
| DL | Deep Learning |
| DPO | Diphenyl Oxalate |
| EAM | Embedded Atom Method |
| HEA | High-Entropy Alloy |
| HEDC | High Energy Density Compound |
| HEDM | High Energy Density Material |
| HTP | High Throughput |
| ICSD | Inorganic Crystal Structure Database |
| IDE | Integrated Development Environment |
| ISP | Specific Impulse |
| JANNAF | Joint Army Navy NASA Air Force |
| JSON | JavaScript Object Notation |
| LAMMPS | Large-scale Atomic/Molecular Massively Parallel Simulator |
| LLNL | Lawrence Livermore National Laboratory |
| MD | Molecular Dynamics |
| MI | Materials Informatics |
| ML | Machine Learning |
| MW | Molecular Weight |
| NASA | National Aeronautics and Space Administration |
| NIST | National Institute of Science and Technology |
| NPSS | Numerical Propulsion System Simulation |
| OI | Optical Isomers |
| PALM | Petroleum Analytics Learning Machine |
| PUG | Power User Gateway |
| REDTOP | Rocket Engine Design Tool for Optimal Performance |
| REST | Representational State Transfer |
| RHEA | Refractory High-Entropy Alloy |
| SADT | Self-Accelerating Decomposition Temperature |
| SLM | Selective Laser Melting |
| SOA | State of the Art |
| SRO | Short-Range Ordering |
| SVE | Statistically Representative Volume Element |
| TBC | Thermal Barrier Coating |
| UTS | Ultimate Tensile Strength |
| VASP | Vienna Ab initio Simulation Package |
| XGBoost | Extreme Gradient Boosting Regression Tree |

We define artificial intelligence as the use of computers to mimic the cognitive functions of humans. When machines carry out tasks based on algorithms in an “intelligent” manner, that is AI. Artificial intelligence is a broader concept than machine learning (DataScienceCentral 2018).

We define machine learning as a subset of AI that focuses on the ability of machines to receive a set of data and learn for themselves, and change algorithms as they learn more about the information that they are processing (DataScienceCentral 2018).

We refer to deep learning as a subset of machine learning. We define deep learning in terms of “deep neural networks”, i.e., neural networks comprising of two or more layers. Deep learning networks need to see large quantities of items in order to be trained (DataScienceCentral 2018).

AI, ML and DL each refer to a suite to techniques (not to a single method), with DL Techniques being a subset of ML techniques, and with ML techniques being a subset of AI techniques.

Supervised learning is a data mining task that involves inference of a function from labeled training data.

Unsupervised learning is a type of machine learning algorithm used to draw inferences from datasets consisting of input data without labeled responses.

Reinforcement learning is an area of machine learning concerned with how software agents ought to take actions in an environment so as to maximize some notion of cumulative reward.

Functional groups are specific groupings of atoms within molecules that have their own characteristic properties, regardless of the other atoms present in a molecule.

Substituent is an atom or a group other than hydrogen in a molecule.

Halide is an entity consisting of two parts. The first part consists of a halogen atom (a fluorine, chlorine, bromine, iodine or astatine atom). But the other part consists of an element or a radical that is less electronegative (or more electropositive) than the halogen atom.

### 2. Best Mode of the Invention

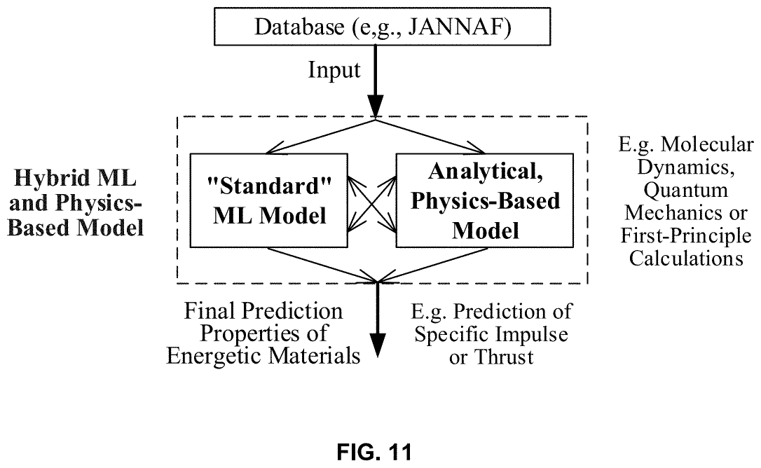

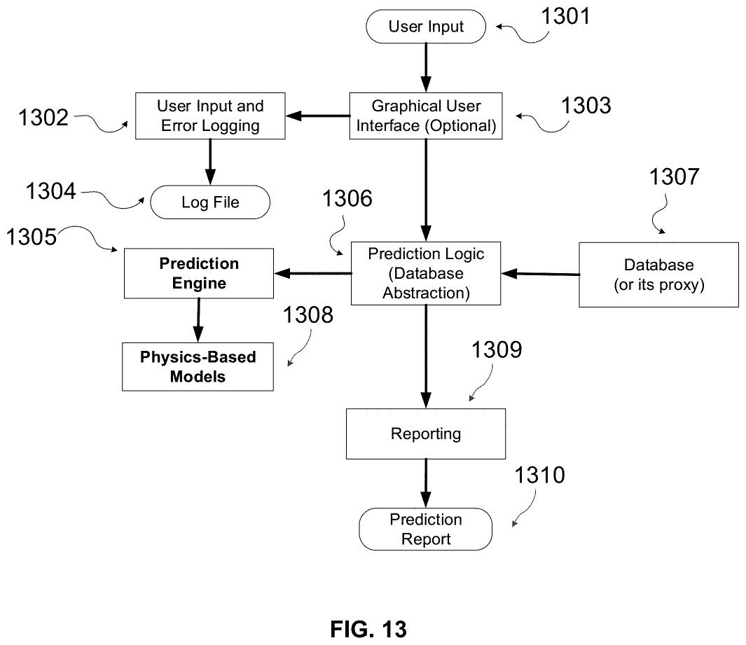

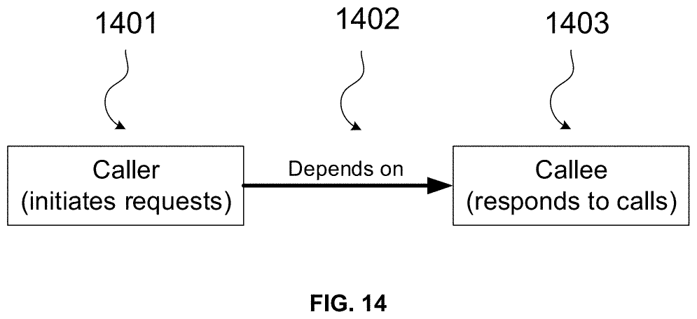

FIG. 11, FIG. 13 and FIG. 14 capture the best mode contemplated by the inventors, according to the concepts of the present invention.

### 3. System Structure at a High Level

FIG. 13 presents a dependency diagram for the overall system architecture. The architecture has been devised largely based on the requirements for the prediction engine. While the architecture may come across as simplistic, it has been artfully crafted such as not to contain any loops. This offers great value in terms of significantly expediting the process of confining the source of certain behavior (desired or undesired) to given modules. FIG. 14 presents the relationship, per the dependency diagram in FIG. 13 between the caller, that initiates a request, and the callee, that responds to a call.

With the graphical user interface and prediction logic being optional, the architecture supports both embedded (plugin or web service) and integrated applications of the prediction engine.

### 4. User Interface

The GUI is assumed to be based on the traditional Model-View-Controller model (Steingrimsson 2017). The GUI may be integrated into a host application, may be executed through a plugin, or may run through a web interface.

### 5. Database System

The purpose of the database system is to enable prediction or filtering of molecules to determine potential HDEM candidates for laboratory synthesis and further considerations. The desired content and structure of a database, for use in conjunction with a material science application, depends on the material properties that one is trying to predict.

### 5.1 General Assumptions

The typical use case assumes a relational database, such as SQL. In order to be most useful, the data needs to be collected into a single repository and have consistent format.

An in-memory implementation of the database stores as inputs vectors, \tilde{x}'s, capturing the sources affecting the output quantity of interest, \tilde{y}. The in-memory database also stores the aforementioned output quantity. For further information, refer to the generic system model in Eq. (1). It is preferable that the database supports an in-memory mode. The prediction engine may require millions of comparative operations. Without an in-memory mode, every comparison may require an I/O call. This may introduce significant latency.

### 5.3 Extendable Solution for Importing Content from Disparate Databases: SQL, JSON and REST

A key challenge in applying ML algorithms to data from materials science or chemistry is that the data may come in many formats. Determining how to featurize and utilize different materials data formats so that prior data can be used as training data for ML algorithms can be difficult. Feature engineering, including extraction, transformation, and selection, is critical for improved ML accuracy. To fully realize data analytics and machine learning tools for materials development, it may be necessary to transform various raw data inputs into information-rich features suitable for modeling. In case of energetic materials, it may be necessary to fully define the physio-chemical data sets suitable for the molecular engineering involved. Hence, it may be necessary to unify possibly disparate data sets into a consistent format that can be utilized by the prediction engine.

A SQL server may be an ideal solution for supporting data from disparate databases, for use by the prediction engine. A SQL server, such as a MySQL server, allows one to import and translate many data types. It allows one to import data from any database supporting the SQL language, search for data of interest, export into a standardized format, and import into the prediction engine.

JSON data is represented in a logical, organized and easy-to-access manner. JSON can contain multiple levels of objects, arrays and various field data. JSON is supported in order to provide open access to material data bases, such as the Citerine's JSON-based database (CitrineResearch 2019). The Citrine database offers an API providing convenient access for users.

PubChem is the world's largest collection of freely accessible chemical information. Chemicals can be searched for by name, molecular formula, structure, and other identifiers. Chemical and physical properties, biological activities, safety and toxicity information, patents and literature citations can be identified along with other information. PubChem data can be downloaded programmatically using various programmatic access routes including E-Utilities, Power User Gateway, PUG-SOAP, PUG-REST, PUG-View and a PubChemRDF REST interface (PubChemDatabaseAPI 2021).

The NIST database contains chemical and physical properties of hundreds of molecules, including boiling and melting temperatures, heat of formation, heat capacity, etc. (NISTdatabase 2021). But the NIST database does not contain the properties of mixtures (NISTdatabase 2021).

A subset of the NIST database refers to inorganic crystal structures. The NIST ICSD database is the world's largest database for completely identified inorganic crystal structures. In case of solids (crystals), data files can be obtained from the ICSD database. Potentials for molecular dynamics simulations can be obtained through the NIST's Interatomic Potentials Repository (NISTpotentialRepository 2021). Similar to PubChem, the ICSD database provides a standard RESTful API for programmatic access (ICSDdatabaseAPI 2021).

### 5.4 Custom Python Interface in Case of the JANNAF Propellant Databases

Alternatively, the data may originate from the databases maintained by the Joint Army Navy NASA Air Force Interagency Propulsion Committee (JANNAF 2021). This includes the Solid Propellant Database, the Propellant & Explosive Ingredients Database, the Liquid Propellants & Fuels Database, the Liquid Rocket Engine Database and the Spacecraft Chemical Propulsion Database. Thermodynamics data from the JANNAF database can be extracted using a custom Python interface (JannafThermoChem 2021). Files are downloaded from NIST servers as needed and then cached locally. The JANNAF thermodynamic data can be used to compute properties of more than 1,200 substances from 0 K to 2,500 K.

### 5.5 Mechanism for Supporting Multiple Data Formats: ETL Extract, Transform and Load

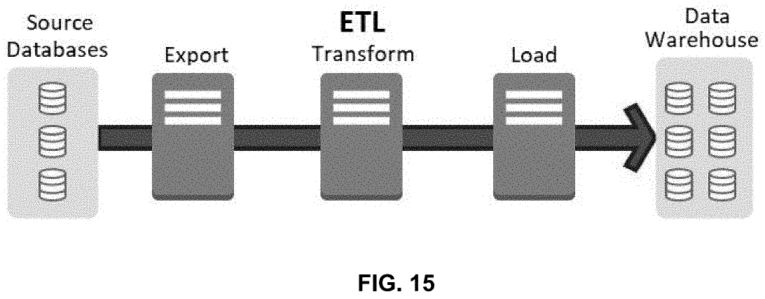

ETL is a generic principle representing three database functions (extract, transform and load) that are combined into a single tool to pull data out of one database and place into another database (see FIG. 15). Extract is the process of reading from a database. In this stage, the data is collected, often from multiple and different types of sources. Transform is the process of converting the extracted data from its previous form into the form it needs to be in so that it can be placed into another database. Transformation occurs by using rules or lookup tables or by combining the data with other data. Load is the process of writing the data into the target database. Traditional symmetric multi-processing data warehouses use an ETL process for loading the data. Ref. (CodeBurst 2019) shows how to Extract data from mysql, sql-server or Firebird, Transform the data, and then Load into a SQL server (a data warehouse) using python 3.6.

### 5.6 Preparation of Data Sets for Prediction Implications of Incomplete Data

Ref (AgrawalDeshpande 2014) suggests that one may be able to employ standard, off-the-shelf, free or open-source machine learning libraries, such as TensorFlow (TensorFlow 2020) or scikit-learn (SciKit-Learn 2020), and obtain reasonably accurate prediction results, assuming one has access to data sets that are clean, usable, abundant and trustworthy (and do not contain outliers).

However, in practice material scientists usually find themselves operating in a realm of limited data.

Hence, in practice, it may be of importance to incorporate physics-based models, in order to extract the most out of the (usually limited) data sets available.

### 6. Prediction Database Logic

The prediction database logic serves as an interface, or abstraction layer, between the prediction engine and the database. The prediction database logic can provide the ability to store internal data structures in memory and later archive in a data base (e.g., by saving on a hard disk drive or a flash drive). The prediction database logic can represent data in a format convenient to the prediction engine.

### 7. Prediction or Optimization Engine

Propellant selection is usually based on many factors. An ideal propellant rarely exists: A large number of propellant candidates usually results from the need to make difficult trade-offs among performance, storability (stability), physical characteristics, handling, safety, cost, and availability. These parameters need to be considered for specific applications and their degree of importance can vary greatly (ForbesForrest 2003).

Generally speaking, a good propellant needs to have high specific impulse, relatively high density, high combustion temperature, high heat of formation, and low molecular weight of products (of the exit gases). More specifically, good HEDMs are expected to provide high detonation performance (maximum detonation velocity D, maximum detonation pressure p_{C,J}, and maximum heat of explosion Qₘₐₓ) with simultaneous maximum stability, which includes minimal chemical degradation upon storage, no phase transition to a polymorph with an inferior performance, and no initiation upon accidental mechanical impact, friction, and non-mechanical stimuli, such as exposure to light, to irradiation in a non-visible spectral range, to electrostatic discharge, etc. (HuangLiTanWen 2021).

The prediction engine, also referred to as the optimization engine, is able to guide a decision on an energetic material to the best candidate. A designer does not have to base the selection of an energetic material exclusively on the outcome of the prediction or optimization engine, although one can. The machine learning may be combined with experimental verification, human insight, and other sources, for purpose of arriving at the best overall candidate. In the context of material science, we consider the purpose of a prediction engine more or less the same as that of an optimization engine. Here, one is typically attempting to identify (predict) the material properties yielding, e.g., the highest specific impulse, optimize for the highest specific impulse.

### 7.1 General Approach to Prediction of Properties for Energetic Materials of Interest

### 1. Structure of a Generic System Model

We assume a generic system model:

\tilde{y}=f(\tilde{x}) (1)

The input vector, \tilde{x}, can be considered as the definition of a feature set comprising of parameters related to the molecular weight of exit gases, chemical energy (heat of formation or heat of reaction), density, viscosity, reactivity, temperature of combustion, etc., essentially all the sources that can impact the output quantity of interest, \tilde{y}. The transformation, f(·), can be a non-linear function of the input, \tilde{x}. We present artificial intelligence, regression analysis and supervised learning as options to construct (train) the system model.

### 2. Input Properties of Interest (Case of Liquid Propellants)

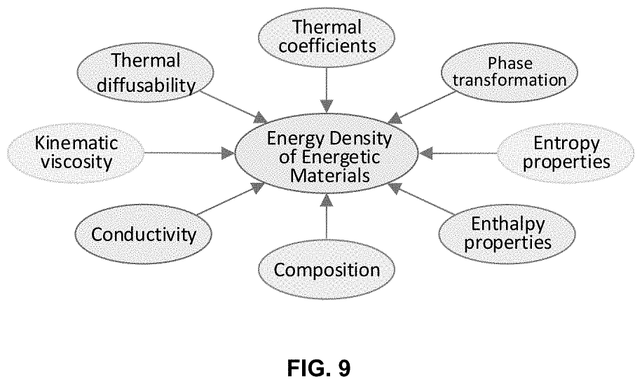

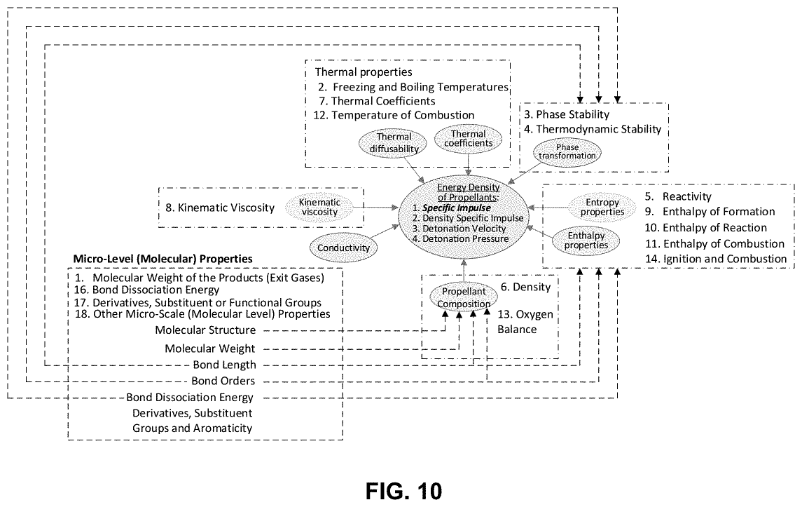

Machine learning fundamentally is a correlative technique. FIG. 9 and FIG. 10 capture key associations between the pertinent inputs and the corresponding outputs. The listing below constitutes a more detailed elaboration of the inputs from FIG. 9 and FIG. 10.

### 1. Chemical Content in Stoichiometry

The structural composition is the primary feature that determines the thermal stability, ignition behavior (sensitivity) and biological toxicity of propellants. The energy density can also be associated with the chemical content which has a significant impact on the heat of combustion of a propellant. For instance, the amounts of carbon in the propellant have a substantial impact on the heat of combustion and the gaseous CO₂ necessary for spacecraft propulsion. This suggests that the content of essential elements like carbon, nitrogen, hydrogen and some metals may have significant impacts on the energy density of the propellant.

The input descriptors may include C/O, C/N, H/C, halide/H and halide/C ratios. Sometimes the fuel is supplemented with halides or even a metal, such as aluminum, in part to achieve a desired density. To increase the density, sometimes the elements of Al, Li or Be are added to the propellants.

### 2. Molecular Weights of the Products (Exit Gases)

Since propulsive energy is produced in the form of hot gases, optimized performance results from maximizing the flame temperature and volume of gases produced per unit weight of the reactants. Low molecular weights for the exit gases are desired, because the acceleration of the exit gases, and the thrust of the rocket that results, is inversely proportional to the MW (see Eq. (35)).

One seeks to produce gases with low molecular weight, such as water (H₂O 2O) or carbon dioxide (CO₂), in the output products. But gases like hydrogen or carbon monoxide are not desirable, because these gases will oxidize. You want to produce some stable gases, such as water and CO₂, at the end, so that you have exhausted all of the energy that you can retrieve from the fuel.

The number of moles of gas produced by per gram of explosives (N) can be estimated as

$$N = \frac{n}{M} \qquad ( 2 )$$

where n is the number of moles of gaseous products and M is the molecular weight of the compounds (KamletJacobs 1968).

### 3. Freezing, Melting and Boiling Temperatures: Temperature Range for Liquid Phase

The operational environment of a missile or a spacecraft dictates the requirements for the freezing, melting and boiling points. Military tactical weapons are often designed for a temperature range of −65- 160° F. (219.3-344.4 K) for deployment. A low freezing point for the fuel allows rockets to operate in cold weather. Some fuels need to be kept under cryogenic conditions, due to cost concerns. Low melting temperature is desired for many ionic liquids, in order to keep them in the liquid form. This helps with storing, handling and ease of transport (with keeping the propellant in liquid condition). However, high boiling or decomposition temperature is usually desired.

### 4. Phase Stability of a Propellant

Propellants need to be storable for the time period required without decomposition, gas formation. or corrosion attack on the material of the storage container. Decomposition or residue formation at high temperatures can adversely affect regenerative cooling. Propellants should also be stable to mechanical and hydrodynamic shock and adiabatic compression.

### 5. Thermodynamic Stability of a Molecule (Thermal Stability)

There is a trade-off between the detonation performance and thermodynamic stability of HEDM molecules (HuangLiTanWen 2021). One does not want the propellant to trigger upon slightest change in environmental condition, but one still expects the propellant to be ignitable. Impressive detonation performance of HEDMs requires large energy difference between the reactants and the reaction products, whereas the high stability of HEDMs relies on a sufficiently high energy barrier to prevent the uncontrolled initiation of such reactions (JiaoXiongZhang 2018).

Other properties, such as bond lengths, bond dissociation energies and bond orders, also provide essential insights about stability, which is critical for packaging and storage, as well as about sensitivity of a molecule for use as a fuel.

The thermal stability and sensitivity of HEDM compounds can be evaluated by identifying the bond where thermolysis is likely to be triggered. This is achieved by making use of molecular structure properties, such as bond length, bond dissociation energy and bond overlap populations, to determine the weakest bond which may be the trigger bond during the thermolysis.

The self-accelerating decomposition temperature (SADT) is the lowest temperature (in air at 1 atm) which self-reactive substance undergoes an exothermic reaction. By comparison, chemical stability refers to any change in material via reaction. Thermal stability is due to a temperature change (thermal change), whereas chemical stability is due to a chemical reaction. The two do overlap, since increase in temperature can initiate a chemical change.

### 6. Reactivity

Mild corrosion of tanks and components and deterioration of seals and gaskets can result in propellant contamination and clogging of filters and small passages. Reactivity with moisture and air invokes the need for unique transfer techniques. Spills and leaks of reactive propellants can constitute safety hazards. A reactive fuel and oxidizer combination is needed for smooth and efficient combustion.

In many of propellant applications, one may be interested in the intrinsic reactivity with an oxidizer, in addition to the detonation velocity.

### 7. Mass Density of a Propellant (p)

High fuel density is desired in order to accommodate a large amount of propellant in a given volume on a spacecraft. High propellant density allows the use of smaller tanks, thus minimizing the overall vehicle inert weight (which improves the mass fraction). If the fuel is of low density, you need a lot of volume to mitigate that. And that may not be good for operational and design constraints.

The density of C, H, N, O molecular crystals, p, can be estimated as (PolitzerMartinezMurray 2009) and (BulatToro-Labbe 2010)

ρ=β₁(M/Vₘₒₗ)+β₂(νσₜₒₜ²)+β₃ (3)

where M is the molecular mass and Vₘₒₗ is the volume of the isolated gas phase molecule that is enclosed by the 0.001 au contour and its electronic density. The constants, β₁, β₂ and β₃ have the values 0.9183, 0.0028 and 0.0443, respectively. The term νσₜₒₜ² reflects strengths, variabilities and degree of balance of positive and negative electrostatic potentials computed on surfaces of isolated molecules, and is calculated by WFA-SAS code proposed by (BulatToro-Labbe 2010).

In case of bipropellants, the density, ρ_{bipropellent}) can be estimated as (Pakdehi 2017)

$${ρ}_{bipropellent} = \frac{( 1 + r ) \times {ρ}_{o} \times {ρ}_{f}}{{ρ}_{o} + r \times {ρ}_{f}} \qquad ( 4 )$$

where r represents oxidizer-to-fuel ratio, ρₒ is density of the oxidizer and ρ_{f} density of the fuel.

Related to the mass density, ρ, is the molar volume, or molecular volume, Vₘ, defined as

Vₘ=M/ρ. (5)

### 8. Thermal Coefficients Thermal Conductivity (K)

A propellant with low coefficient of expansion can be used over a larger portion of its liquid range without undue performance variation. In terms of heat transfer properties, high specific heat, high thermal conductivity are desired.

### 9. Kinematic Viscosity (η) and Surface Tension (γ)

Low viscosity helps in terms of easy transport of fuel in tanks and injection into thruster. Low viscosity fluids will have less pressure drop through the feed system and thrust chamber injector, permitting a smaller pump or a lightweight pressurization system to be used. Lower viscosity propellants are also easier to pump.

Similarly, surface tension tanks exploit the surface tension properties of propellants to control, manage and deliver gas-free oxidizers or fuel, especially under micro-gravity conditions.

### 10. Enthalpy, i.e., Heat, of Formation (ΔH_{f})

The standard enthalpy of formation (also referred to as the standard heat of formation) of a compound, ΔH_{f}, describes the energy required to form the compound. The enthalpy of formation is defined as the change in enthalpy during the formation of 1 mole of the substance from its constituent elements, with all substances in their standard states. For specifics, refer to (WikipediaEnthalpyFormation 2021). For ionic compounds, the standard heat of formation is equivalent to the sum of several terms included in the Born-Haber cycle. The formation of lithium fluoride, LiF,

$$L {i}_{( s )} + \frac{1}{2} {F}_{2 ( g )} = L i {F}_{( s )} \qquad ( 6 )$$

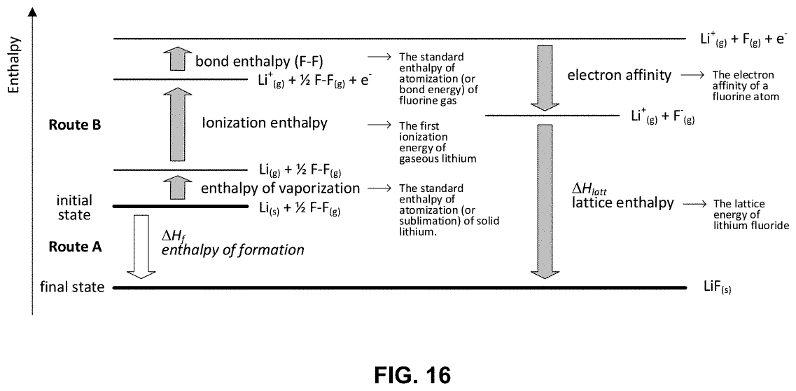

may, for example, be considered as the sum of several steps, each with its own enthalpy (see FIG. 16).

### 11. Enthalpy, i.e., Heat, of [Any] Reaction (ΔH_{r×n})

The standard enthalpy of reaction (also referred to as the standard heat of reaction), ΔH_{r×n}, refers to the heat released (or absorbed) during any chemical reaction. The heat of combustion is the sum of the heat of formation of the products minus the sum of heat of formation of the reactants. In other words, ΔH_{r×n} is defined as the difference between total reactant and total product molar enthalpies due to a chemical reaction, from reactants in their standard states to products in their standard states:

ΔH_{r×n}=Σ_{p=products}νₚΔH_{f,p}−Σ_{r=reactants}νᵣΔH_{f,r} (7)

where νₚ and νᵣ represent the stoichiometric coefficients of each product p and reactant r, ΔH_{f,p} denotes the heat of formation for product p and ΔH_{f,r} the heat of formation for reactant r. A very high negative heat of reaction (energy release) is desired.

### 12. Enthalpy, i.e., Heat, of Combustion [Reaction] (ΔH_{comb}), and Heat of [Combustion] Explosion

The enthalpy of combustion, ΔH_{comb}, is highly related to (a special case of) the enthalpy of reaction (WangXuZhang 2019). The heat of combustion is the heat released (or absorbed) during a combustion reaction. The heat of combustion can be calculated from the heat of formation of the gaseous products and the fuel (reactants) as follows:

ΔH_{comb}=Σ_{r=reactants}NᵣΔH_{f,r}−Σ_{p=products}NₚΔH_{f,p} (8)

Here, the definition of ΔH_{f,r} and ΔH_{f,p} is the same as for Eq. (7). But Nₚ and Nᵣ (mol) represent the moles of products and reactants, respectively. Consequently, increases in the heat of formation of a propellant results in a rise in the heat of reaction (combustion), and thus increases in the specific impulse. Similarly, higher density contributes to higher heat of combustion.

More specifically, the enthalpy (heat) of explosion refers to the enthalpy (heat) of combustion for a combustion resulting in an explosion.

### 13. Temperature of Combustion (T_{c})

The temperature of combustion is critical in determination of the specific impulse. The temperature of combustion, T_{c}, is related to the initial temperature of a detonation process, T₀, and the heat of combustion, ΔH_{comb}, through (WangXuZhang 2019)

$${T}_{c} = {T}_{0} - \frac{Δ {H}_{c o m b}}{{C}_{p , g a s}} \qquad ( 9 )$$

where C_{p,gas} represents the total heat capacity of the gaseous products resulting from the detonation. The heat of combustion is assumed to remain constant over the temperature range (Tₒ-Tₐ). And pressure in the combustion chamber is assumed to remain constant at the steady state conditions.

We consider the terms combustion temperature and detonation temperature as synonymous.

### 14. Oxygen Balance (OB) and Oxidizer-to-Fuel Ratio (O/F)

The oxygen balance reflects the degree of which a propellent can be oxidized. A positive oxygen balance signals that the propellent has more oxygen than needed for complete oxidation (combustion), while negative oxygen balance means the propellant has less oxygen than needed. In general, the oxidation balance is calculated as (WikipediaOB 2021)

$${OB}_{100} = \frac{- 1600}{Mol . weight of compound} \times ( 2 X + ( \frac{Y}{2} ) + M - Z ) \qquad ( 10 )$$

where X represents the number of carbon atoms, Y the number of hydrogen atoms, Z the number of oxygen atoms, and M the number of metal atoms (that can produce metal oxide).

For example, in case of TNT (C₆H₂(NO₂)₃CH₃),

Molecular weight=7*12.01+5*1.008+3*14.01+6*15.996=227.1, (11)

so

$${OB}_{100} = \frac{- 1600}{227.1} \times ( 2 * 7 + ( \frac{5}{2} ) + 0 - 6 ) = 73.98 \% \qquad ( 12 )$$

The oxygen balance, therefore, is related to the chemical composition of the fuel.

Along these lines, the oxygen balance can be considered as the proportion of oxygen in the structural formula of energetic molecules. An explosive needs to have enough oxygen during the combustion process to convert all carbons into gaseous carbon dioxide, all nitrogen in dinitrogen and all hydrogen in water by products. If the amount of oxygen present in an energetic molecule is insufficient for complete oxidation, a negative oxygen balance will result and undesired byproducts will be formed such as carbon monoxide. For example, when TNT (C₆H₂(NO₂)₃CH₃) is detonated, it is oxidized such as to form gaseous products. If the reaction products are fully oxidized to form gaseous CO₂, H₂O and N₂ (KamletAdolph 1979)

$${OB}_{100} = \frac{100 ( 2 {n}_{c} - {n}_{H} - 2 {n}_{c} )}{M} \qquad ( 13 )$$

Here, n_{O}, n_{H}, and n_{C} represent the numbers of O, H, and C atoms, respectively. According to (WangXuZhang 2019), the oxygen balance can be used to roughly predict the impact sensitivity of explosives. The heat of combustion is also affected by the oxidizer-fuel mass ratio.

Oxidizer-to-fuel ratio refers to the mass ratio between fuel and the oxidizer. The O/F ratio can be captured in the form of the stoichiometric ratios, that is the amount of oxidizer needed to fully oxidize the fuel or based on the optimal ratio for each bipropellant engine design.

The oxygen balance and the oxidizer-to-fuel ratio refer to separate concepts. There is no general correlation known related the OB % and the O/F.

### 15. Ignition and Combustion

Chemical propellants need to be easily ignitable and have good combustion properties. Hypergolic ignition offers high reliability, a restart capability for multi-burn missions and eliminates the weight of a separate ignition system. In space, secondary ignition sources tend to be unreliable. Catalytic decomposition of monopropellants is a valuable attribute.

### 16. Ignition-Delay Time

Ignition-delay time for bipropellants specifies, under some standard conditions, the time it takes for the fuel and the oxidizer to ignite. In general, the ignition-delay time represents the time between fuel injection and combustion. It is important to have a good sense of the ignition-delay time for the propellants under consideration.

Thermal stability refers broadly to ay breakdown mechanism. One way for fuel to decompose is through ignition. But fuel can degrade by other mechanisms too.

### 17. Bond Dissociation Energy, Ionization Energy and Bond Energy

The bond dissociation energy is a key metric for characterizing the thermal stability of propellants. The bond dissociation energy refers to the energy needed to break a chemical bond. The BDE is fundamental to understanding of bond strengths. The enthalpy of reaction of

A−B_{(g)}→A·_{(g)}+B·_{(g)} (14)

is the BDE of the bond A-B by definition (DuHanLiuWu 2019).

Ionization energy refers to the energy needed to ionize an atom or a molecule, that is removing an electron.

### 18. Derivatives, Substituent or Functional Groups, or Aromaticity

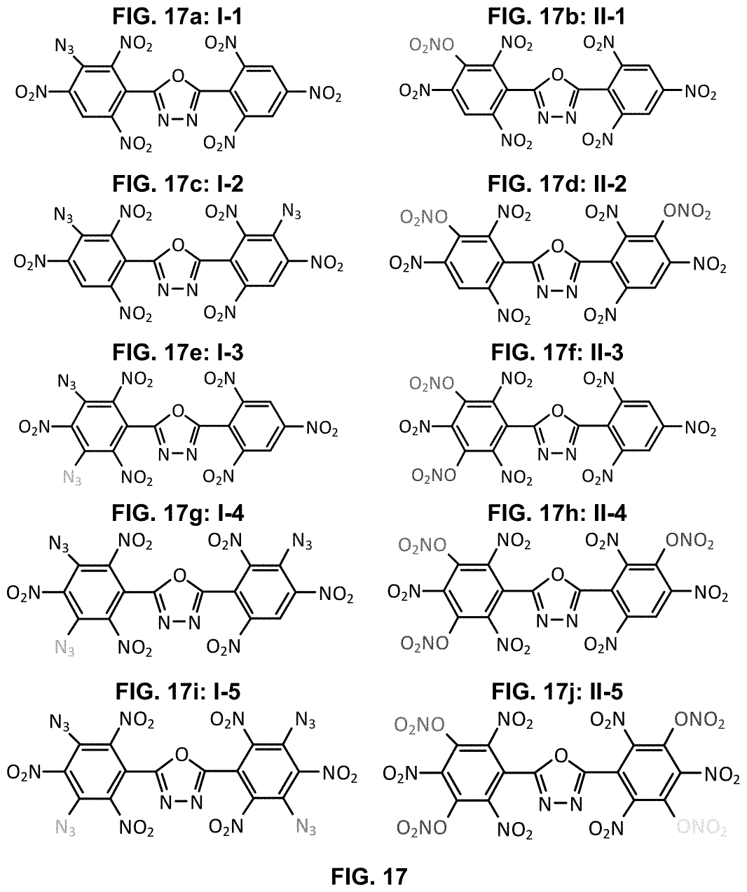

FIG. 17 presents five derivative classes of one HEDC compound, diphenyl oxalate. Derivatives I-1 to I-5 contain one, two or three nitride (N₃) groups added to the main DPO molecule. They are referred to as azido derivatives of the main molecule. Similarly, derivatives II-1 to II-5, which have one, two, three or four groups of ONO₂ added to the main molecule, are referred to as nitrate derivatives.

A functional group refers to an identifying chemical group (a lowest level demoninator). Functional groups are specific groupings of atoms within molecules that have their own characteristic properties, regardless of the other atoms present in a molecule. Adding or changing a functional group is a way to systematically change the chemical properties of a molecule. Chemists use this as a design tool when designing new molecules. Many studies will change out one functional group and see how that affects the properties. Table 2 provides a definition of the key functional groups of interest. Machine learning can help identify the functional groups that are favorable for high energy density (e.g., high specific impulse).

Functional groups are very important property in chemistry and can have large impact on molecular interactions and reactions. The number and type of functional groups present can have major impact on the heat of combustion or specific impulse of energetic materials. But interpreting functional groups may not always be easy. For example, CH3-CH2-CH2-COOH would be an acid (the functional group would be acid), although on the far end there is an alkane.

TABLE 2
Definition of the key functional groups of interest. Here, R refers
to some alkane chain for example CH₃—CH₂—CH₂.

| Functional | |
|---|---|
| Group | Formula/Explanation |
| Acid | R—(C═O)—OH |
| Alcohol | R—OH |
| Aldehyde | R—HC═O |
| Alkane | Alkane is a carbon chain with only single bonds |
| | (e.g., CH₃—CH₂—CH₂—CH₃) |
| Amide | R—(C═O)—NH₂ |
| Amine | —NH₂ |
| Azido | —N₃ |
| Ester | R—(C═O)—O—R |
| Ether | R—O—R |
| Fluoro | —F |
| Hydroxy | R—OH |
| Imine | R—(HCN)—R |
| Ketone | R—(C═O)—R |
| Methyl | —CH₃ |
| Nitramines | R—NHNO₂ |
| Nitrides | Molecule where N has −3 charge (for metal M that |
| | would be MN, but for alkaline earth metals Ca₃N₂) |
| Nitrile | R—CN |
| Nitro | R—NOO |
| Thiol | R—SH |

Aromaticity in chemistry refers to a property of cyclic (ring-shaped), planar (flat) structures with pi bonds in resonance (those containing delocalized electrons) that gives increased stability compared to other geometric or connective arrangements with the same set of atoms (WikipediaAromaticity 2021). The presence of polycyclic aromatic rings on a molecular backbone has been reported to affect the energy density of propellants (SavoskinKapkan 2007), (YuanZhangTao 2020).

Machine learning can help characterize the impact of different derivative formation, substituent or functional groups on the energy density and performance of the energetic materials. 19. Other Micro-Scale (Molecular Level) Properties, Related to Bond Length or Molecular Structure

Bond lengths can be estimated using first-principle calculations. As the length of a bond grows longer, the interaction between the atoms becomes weaker, so it takes less energy to break the bond. The bond easiest to break can become a trigger point for detonation. And if a bond is too easy to break, then the molecule can be unstable, resulting in a suspect propellant. Detonation properties can be impacted by the number of nitro, azido, nitrates, hydroxy substituent groups present.

Bond orders refers to the number of covalent bonds shared between two atoms. A single bond is formed (bond order=1), when the atoms share one pair of electrons. A double bond (bond order=2) is formed, when two electron pairs are shared. Sharing of three electron pairs results in a triple bond (bond order=3).

Bond overlap population is a measure of the shared electronic density between two atoms forming a chemical bond.

### 3. Additional Input Properties of Interest (Case of Propellants or Explosives in Solid Form)

### 20. Cohesive Energy (eV/Atom)

Cohesive energy refers to the energy gained by arranging the atoms in a crystalline state, as compared to a gas state. In (KangLiu 2020), the cohesive energy, averaged over all constituent elements, and the oxygen balance are identified as the critical descriptors.

### 4. Output Properties of Interest

Similar to the input properties, the output properties of interest are presented in context with FIG. 9 and FIG. 10. Many possibilities exist for prediction. One, e.g., can predict thermodynamic stability of a molecule, given the molecular structure together with an initiation point (trigger) for thermolysis. But generally speaking, we are looking to maximize the energy density or the energy/mass density (detonation ability, i.e., performance), measured in terms of (1) Specific Impulse, (2) Density Specific Impulse or Relative Specific Impulse, (3) Detonation Velocity and (4) Detonation Pressure.

### 1. Specific Impulse: Thrust per Unit Flow Rate

For rockets, the specific impulse is defined as the rocket thrust per unit flow rate of propellant:

$${I}_{s p} = \frac{F}{m} \qquad ( 15 )$$

The specific impulse is the conventional parameter for theoretical comparison of various propellants and for assessment of the efficiency of actual delivered rocket engine performance compared the theoretical, because it describes how much the rocket thrust (change in velocity) is impacted by burning the fuel. In the English units, m is the weight flow rate in pounds per second. Since the thrust is in pounds, this leaves the unit of specific impulse to be seconds. For a more physical definition, specific impulse can be thought of as pounds of force per pound per second of propellant flow. In any units, the higher the value of specific impulse, the higher the specific performance of the rocket. When a rocket is reported to have a specific impulse of 1000 m/sec, it means that the velocity of gases exiting the nozzle is 1000 m/sec relative to the nozzle.

The specific impulse can be recorded separately for frozen vs. shifting equilibrium. In case of shifting equilibrium, the flow stays in equilibrium through the nozzle. In case of frozen equilibrium, the flow is frozen, so the composition cannot change. Both cases are considered a part of isentropic nozzle expansion (JerrySeitzman 2021).

For an ideal, completely reacted gas, i.e., no dissociated products, there is a semi-empirical relation that ties the specific impulse to the combustion temperature and the molecular weight of the combustion products (DouglasRapp 1990):

$${I}_{s p} ∝ \sqrt{\frac{{T}_{c}}{{M}_{c}}} \qquad ( 16 )$$

The specific impulse increases with higher combustion temperature (T_{c}) and lower molecular weight (M_{c}) of the combustion products. When you are burning the propellant, you want to produce very light weight compounds from the combustion, e.g., water or carbon dioxide. If the combustion results in heavier products, it means that it is not very efficient.

For additional analytical relationships connecting the thrust and the specific impulse with the underlying thermodynamics, refer to Eq. Eq. in Section 7.2.

### 2. Density Specific Impulse and Relative Specific Impulse

As the density of the propellant decreases, the specific impulse generally increases. Since no single fuel provide both high Iₛₚ and high density, a trade-off exists. To assess this trade-off, a density specific impulse, I_{d}, is introduced (DouglasRapp 1990):

I_{d}=δₚ*Iₛₚ (17)

Here, δₚ represents the ratio of the propellant density to the density of liquid water, i.e., the specific gravity of the propellant. Multiplying with the specific gravity may get rid of the density dependance, according to Eq. (15), at least as first-order approximation, since the mass term in Eq. (15) scales linearly with the propellant density (m∝p). But the mass density of the fuel also impacts the thrust (the force term in Eq. (15)), since F_{gravity}≈(rocket mass)×gravity. Further, higher density contributes to higher heat of combustion, according to Eq. (8), which also affects the thrust. For additional information on the impact of the density on the thrust, refer to (SavoskinKapkan 2007).

In addition to the specific impulse, a few normalized version, referred to as relative specific impulse, have also been introduced. Ref. (GagneMcDevittHitt 2018) introduces a theoretical relative specific impulse that is the theoretical maximum achievable specific impulse for each solvent considered normalized by the maximum achievable specific impulse of 85% hydrogen peroxide and water. Ref (Türker 2013) introduces a relative specific impulse which is the specific impulse of explosive 1 to the specific impulse of explosive 2.

### 3. Volume of Gaseous Products (V_{g})

The volume of gases produced during an explosion provides information on the extent of the work done by the energetic materials (KangLiu 2020).

### 4. Detonation Velocity (D) or Exhaust Velocity (νₑ)

Kamlet and Jacobs showed that the detonation velocities of C—H—N—O explosives at initial densities above 1.0 g/cc may be calculated by means of a simple empirical equation (KamletJacobs 1968)

D=Aφ^{1/2}(1+Bρ₀) (18)

φ=M N^{1/2}Q^{1/2} (19)

Here, D represents the detonation velocity, N is the number of moles of gaseous detonation products per gram of explosives, M is the average weight of these gases, Q is the chemical energy of the detonation reaction (−ΔH₀ per gram), ρ₀ is the initial density, A=1.01 and B=1.30.

### 5. Detonation Pressure (P)

Kamlet and Jacobs similarly presented a simple empirical equation for estimating the detonation pressure, P, for C—H—N—O explosives (KamletJacobs 1968):

P=Kρ₀²φ (20)

K=15.58 (21)

The values of M, N and Q may be estimated from the H₂O—CO₂ arbitrary decomposition assumption. Hence, the calculations require no input information other than the explosive's elemental composition, heat of formation and loading density.

### 6. Detonation Power (Pₑ)

During detonation, heat and gases are liberated. The volume of gaseous products, Vg, and the heat of combustion, ΔH_{comb}, can both be calculated independently. Their values can then be combined to produce an estimation of the power resulting from an explosion (KangLiu 2020):

Pₑ=V_{g}×ΔH_{comb}. (22)

### 7. Biological Toxicity and Biocompatibility

Although toxicity may not have major impact on performance, it is an important concern for storage and handling. Toxic propellants may require breathing apparatus, special storage and transport infrastructure and extensive personnel protective equipment. AI may provide insights into correlations between the structure of a fuel and its toxicity. Certain structures (groups) or atoms or molecules in a fuel can be very toxic to humans, and need to be avoided. Toxicity is characterized in terms of LD50 and LC50. LD50 is the amount of material in mg per kg., which can result in death of 50% from a group of test animals. LC50, on the other hand, denotes the concentration of a chemical in air (in parts per million) that kills 50% from a group of test animals during the observation period (Pakdehi 2017).

### 8. Flammability and Volatility

Flash point temperature is defined as the minimum temperature to which a liquid must be heated to produce enough vapor to allow a vapor flash to occur in the presence of an ignition source. The fire hazard of a flammable liquid warrants consideration of the temperature of the flammable liquid in relationship to its flashpoint.

### 9. Impact or Friction Sensitivity (Part of Safety and Handling)

Neither the impact nor the friction sensitivity refer to energetic properties directly. These are more of safety and handling parameters. The impact sensitivity represents the minimum impact energy (in J) at which explosion can occur. Similarly, the friction sensitivity captures the amount of friction or rubbing that a compound can withstand, before prematurely exploding.

### 5. General Formulation of Descriptors (Features) of Interest

We capture the input properties of interest above in a vector, X, referred to as Descriptors:

$$\begin{matrix}X = Descriptors = [ Descriptors\_on \_chemical \_ \\ content\_in \_stoichiometry , \\ Descriptors\_on \_molecular \_ \\ weights\_of \_products , \\ Descriptors\_on \_freezing \_or \_ \\ boiling\_temperatures , \\ Descriptors\_on \_phase \_stability , \\ Descriptors\_on \_thermodynanic \_stability , \\ Descriptors\_on \_reactivity \\ Descriptors\_on \_mass \_density , \\ Descriptors\_on \_thermal \_coefficients , \\ Descriptors\_on \_kinematic \_viscosity , \\ Enthalpy\_of \_formation ( Δ {H}_{f} [ kJ / mol ] ) , \\ Enthalpy\_of \_reaction ( Δ {H}_{rxn} [ kJ / mol ] ) , \\ Enthalpy\_of \_combustion ( Δ {H}_{comb} [ kJ / mol ] ) , \\ Temperature\_of \_compustion ( {T}_{c} [ °C . ] , \\ Descriptors\_on \_oxygen \_balance \_or \_ \\ oxygen\_to \_fuel \_ratios , \\ Descriptors\_on \_ignition \_and \_combustion , \\ Descriptors\_on \_ignition \text{-} delay\_time , \\ Descriptors\_on \_bond \_disslocation \_energy , \\ Descriptors\_on \_derivatives \_substituent \_ \\ functional\_groups , \\ Descriptors\_on \_other \_microscale \_properties ] .\end{matrix} \qquad ( 23 )$$

In addition to absolute content, the descriptors on the chemical content in stoichiometry may contain ratios, such as the ratio of carbon-to-hydrogen, the ratio of oxygen-to-nitrogen, the ratio of carbon-to-nitrogen or the ratio of carbon-to-hydrogen (the species-to-species ratios in an organic compound). The descriptors may also capture halides or metals added to the fuel, as noted above, and as further reflected in Eq. (31) and Eq. (32) below.

In regards to more specific formulation of the sub-descriptors, one can structure the descriptor on the derivatives, substituent and functional groups as follows:

$$\begin{matrix}\begin{matrix}Descriptors\_on \_derivatives \_ & = & [ main molecule , \# \_of \_nitride \_ \\ substituent\_and \_ & & groups , \# \_of \_hydroxide \_groups , \\ functional\_groups & & \# \_of \_nitramine \_groups , … ]\end{matrix} & ( 24 ) \\ \begin{matrix}Descriptors\_on \_other \_ & = & [ descriptors\_on \_bond \_lengths \_between \_ \\ microscale\_groups & & atoms , descriptors\_on \_bond \_order , … ]\end{matrix} & ( 25 )\end{matrix}$$

Exact formulation of the descriptors needs to be coordinated in conjunction with results from the machine learning model applied. In case the ML model is looking to maximize the specific impulse, Iₛₚ, the exact formulation of the descriptors may be adjusted such as to minimize the scatter (variance) in the data points on a graph showing the predicted vs. the observed impulse (I_{sp,predict} vs. i_{sp,observed}). One needs to look at the outliers, and analyze the causes for the observed impulse deviating from the predicted one. One needs to adjust the formulation of the descriptors such as to truly account for all of the input sources that impact the variations in the output observed. For a similar approach, aimed at minimizing the variations in the ultimate tensile strength (UTS) observed for high-entropy alloys (HEAs), i.e., minimizing scatter in data on a plot showing UTS_{predict} VS. UTS_{observed}), refer to (SteingrimssonFanKulkarni 2020) and (SteingrimssonFanKulkarni 2021).

The formulation of the descriptors, such as for the derivatives, substituent and functional groups, may also draw upon similar formulations from (SteingrimssonFanKulkarni 2020) and (SteingrimssonFanKulkarni 2021). The intent is to identify from the performance the functional groups that are favorable for high performance. Ref. (SteingrimssonFanKulkarni 2020) and (SteingrimssonFanKulkarni 2021) describe a structurally similar approach for encoding the parameters of processes for heat treatment of HEAs into descriptors for ML analysis. The approach for encoding the parameters of the molecular structures exhibits structural similarity with the approach outlined there for parametrizing the microstructure of alloys.

The descriptors above have been crafted such that there is physical meaning attached to them. We want to avoid scenarios, where ML is capable of predicting the properties of a new molecule with very high degree of accuracy, but without the descriptors used for the correlations having physical meaning. The purpose of the ML analysis is to help chemical scientists and engineers with design of HEDCs. The chemical scientists and engineers usually have good understanding of the underlying physical phenomena. Hence, it is important to understand the physical reasons behind given correlations. The chemical scientists and engineers often want to understand which electronic property, or which fundamental property of the molecule, is leading to the behavior observed. They likely will want to make sure that the correlations observed are consistent with quantum theory.

### 6. Essential Structure and Key Characteristics of the Prediction Engine

We design the prediction engine such as to optimize the output quantities of interest. One usually seeks to maximize the specific impulse, the density specific impulse, the detonation velocity and the detonation pressure, but minimize the toxicity. In case of the specific impulse, the assignment (optimization problem) can be formulated as follows:

$$\begin{matrix}{max}_{x} = [ Specific\_Impuse ( X ) ] = {max}_{x} [ {I}_{S P} ( X ) ] & ( 26 ) \\ {max}_{x} = [ Density\_Specific \_Impuse ( X ) ] = {max}_{x} [ {I}_{d} ( X ) ] & ( 27 )\end{matrix}$$

The method selected for solving these problems will largely depend on the type and extent of the data available (SteingrimssonFanKulkarni 2020) and (SteingrimssonFanKulkarni 2021).

Furthermore, as opposed to crafting a black-box ML model, we want to analytically account for as much of the underlying physics as possible. In this way, one can make the most of the usually limited input data available. The ML algorithms do not need to deduce from the data the model dependence on parameters whose dependence is already known from the underlying physics. Similar rational underlies introduction of the bilinear and trilinear log models of (SteingrimssonFanYang 2021), where temperature is analytically accounted for in the modeling of ultimate or yield strength of a superalloy or a high-entropy alloy. Note the larger number of unknown parameters a model contains, or the more general structure the model has, the more input data is needed for accurately estimation of the model. In the design of energetic material, one is usually operating in a low-data regime.

We will present rational on the type of optimization problem that we are looking to formulate: As opposed to crafting a black-box model, we want to factor in as much of the underlying physics as possible, per FIG. 11. In this way, one can make the most of the limited data available.

In case of the specific impulse, we can analytically account for the model dependance on the temperature of combustion and the density:

$$\begin{matrix}\begin{matrix}Specific\_Impulse ( X ) = {I}_{SP} ( X ) = F / m = ( thrust ) / ( molecular\_weight \_of \_products ) \\ = g ( oxidizer , fuel , \frac{oxidizer}{fuel} ratio , … ) * \sqrt{\frac{temp . \_of \_combustion}{density}}\end{matrix} & ( 28 ) \\ {max}_{x} [ {I}_{S P} ( X ) ] = {max}_{x} [ g ( oxidizer , fuel , \frac{oxidizer}{fuel} ratio , … ) * \sqrt{\frac{temp . \_of \_combustion}{density}} ] & ( 29 )\end{matrix}$$

Here,

X=[oxidizer,fuel,oxidizer-to-fuel ratio,temperature of combustion,density, . . . ]. (30)

The oxidizer and the fuel can be further broken down and parametrized description introduced as follows:

$$\begin{matrix}oxidizer = [ \% carbon in oxidizer , \% hydrogen in oxidizer , \% oxygen in oxidizer , \% nitrogen in oxidizer , \% fluorine in oxidizer , \frac{carbon}{oxygen} ratio , \frac{carbon}{nitrogen} ratio , \frac{carbon}{hydrogen} ratio , … ] & ( 31 ) \\ fuel = [ \% carbon in fuel , \% hydrogen in fuel , \% oxygen in fuel , \% nitrogen in fuel , \% fluorine in fuel , [ similar \% s for other halides ] , \frac{carbon}{oxygen} ratio , \frac{carbon}{nitrogen} ratio , \frac{carbon}{hydrogen} ratio , … ] & ( 32 )\end{matrix}$$

In case of the density specific impulse, we can analytically account for the model dependance on the temperature of combustion and the density, in a similar fashion, and formulate the optimization problem as

$${max}_{x} [ {I}_{d} ( X ) ] = {max}_{x} [ g ( oxidizer , fuel , \frac{oxidizer}{fuel} ratio , … ) * {δ}_{p} * \sqrt{\frac{temp . \_of \_combustion}{density}} ] \qquad ( 33 )$$

### 7. Additional Characteristics of the Prediction Engine In Context with the Underlying Physics

A classic equation for the thrust of a rocket, F, is given by (ForbesForrest 2003):

F=m Vₑ+(Pₑ−Pₐ)Aₑ (34)

Here, m represents mass flow rate of working fluid, Vₑ the exit velocity of the working fluid, Pₑ the pressure of the working fluid at the place the working fluid exits the rocket, Pa the pressure in the surroundings of the rocket and Aₑ the area of the exit of the nozzle (ForbesForrest 2003).

From the principles of thermodynamics and fluid mechanics, an equation can be derived for the exit velocity of the working fluid, using conservation of energy, perfect, gas, and a concept called a reversible adiabatic flow. According to (ForbesForrest 2003), the velocity out of the exit of the nozzle can be written as:

$${V}_{e} = \sqrt{\frac{k}{k - 1} 2 g \frac{R}{MW} {T}_{c} [ 1 - {( \frac{{P}_{e}}{{P}_{c}} )}^{( k - 1 ) / k} ]} \qquad ( 35 )$$

Here, k represents the ratio of specific heats of the working fluid, g is the gravitational constant, R denotes the universal gas constant. MW is the molecular weight of the working fluid, T_{c} represents the absolute temperature of the working fluid at the entrance of the nozzle, Pₑ is the pressure of the working fluid at the exit of the nozzle, and P_{c} denotes the pressure of the working fluid at the entrance of the nozzle.

From Eq. (34) and Eq. (35), several of the driving factors for propellant selection can be recognized. To maximize Vₑ, which according to Eq. (34) maximizes thrust, it makes sense to aim for

1. The lowest value of k.
2. The lowest value of molecular weight.
3. The highest value of T.
4. The highest value of P_{c}.

Motivated by Eq. (34) and Eq. (35), if the specific heat capacity of the working fluids is included in the set of input descriptors, the analytical, physics-based optimization model can be extended further:

$${max}_{x} [ {I}_{S P} ( X ) ] = {max}_{x} [ g ( oxidizer , fuel , \frac{oxidizer}{fuel} ratio , … ) * \sqrt{\frac{temp . \_of \_combustion}{density} [ 1 - {C}^{( k - 1 ) / k} ]} ] \qquad ( 36 )$$

The specific heat capacities comprising k may be obtained from the NIST database (NISTdatabase 2021), (ICSDdatabaseAPI 2021). The factor C represents the ratio of the pressures at the exit and entrance of the rocket nozzle (ForbesForrest 2003). The factor C resembles the Peclet number.

### 7. More on Structure of the Prediction Engine (in Context with “Standard” ML)

We present a scalable solution for deriving the system model, one that accounts for the application at hand and the input data available. In the case of a small set of input data, we present regression as a suitable tool for deriving (constructing) the system model. But for a large set of input data, say, hundreds, thousands, or millions of (\tilde{x}, \tilde{y}) duplets, we present feed-forward neural networks as a suitable tool for constructing the system model.

Both Matlab and Python offer standard libraries and toolboxes for multi-linear regression and for neural networks (for “standard” machine learning). In addition, Matlab and Python offer specific libraries for deep neural networks and for reinforcement learning (MatlabMachineLearning 2021), (MatlabDeepLearning 2021), (MatlabReinforcementLearning 2021), (MatlabDeepLearningHDL 2021), (PythonKeras 2021), (PythonNumPy 2021), (PythonSciKitLearn 2021), (PythonCaffe 2021), (PythonTorch 2021), (PyQLearning 2021), (PyTensorForce 2021) and (PyRLcoach 2021).

For description of a prediction engine employing “standard” ML solutions, refer to (SteingrimssonFanKulkarni 2020) and (SteingrimssonFanKulkarni 2021). These references also describe “non-standard” ML solutions, such as “forward” prediction of observed material properties from the input drivers as well as “backward” inference of input drivers given the observed properties.

For description of unique aspects of the prediction engine, including incorporation of physics-based prediction in a hybrid ML scheme as well as joint optimization, refer to Sections 7.2 and 7.3 below.

### 7.2 More on Specific Approaches to Prediction Incorporating Physics-Based Modeling Hybrid ML and Physics-Based Prediction

One of the primary, unique aspects of the innovation involves incorporation of physics-based models into standard ML, per FIG. 11. There can be great benefits derived from combining ML with physics-based modeling approaches for improving accuracy in prediction of properties of energetic materials, since both approaches have their pros and cons, as Table 3 illustrates. This invention combines the strengths of both approaches, in order to make up for their individual weaknesses. The invention makes the most of the—usually limited input data available as well as of the physics-based simulation tools available.

TABLE 3
Comparison of the strengths and weaknesses of standard machine learning (an ML only
approach) with physics-based modeling.

| Approach | Strength | Weakness |
|---|---|---|
| Machine Learning Only | Fast prediction | Limited by data availability |
| | Good for initial screening | (neighbors close to target) |
| Physics-Based | Usually accurate | Can be slow |
| Modeling Only | Less reliant on extensive database | |
| Hybrid Machine Learning & | Tunable speed/accuracy | Not quite as fast |
| Physics-Based Modeling | Relatively fast and accurate | as ML only |

Assuming a physics-based model for modeling the performance of an energetic material is a very complex, but relatively well-behaved function, i.e., without major jumps or discontinuities, one can in principle decompose such a well-behaved function into a linear combination of kernel (basis) functions. Kolmogorov theorem proves that such a decomposition exists, but does not provide the decomposed entities or tell you how to carry out the decomposition (SteingrimssonFanKulkarni 2021).

For formal overview on principal component analysis representation, dual (regularization) representation, and corresponding sparse representations of a functional model, refer to (PoggioGirosi 1998). In this context, if the well-behaved function contains exponentials, it may not be decomposable into a sparse representation. To accurately replay the signal, in this case, you may need to include a lot of harmonics.

Motivated by Kolmogorov's theorem (SteingrimssonFanKulkarni 2021), a neural network can be viewed as truncated representation of nonlinear regression conducted over the set of basis functions selected. There is a trade-off between the selection of the basis functions and the size of the network (the number of harmonics needed to be included for accurate representation of the input). Selection of the kernel (basis) functions comes down to how to obtain a sparse representation of the decomposition over the kernel functions selected. The better you understand the underlying physics, the fewer number of kernel functions you may need to accurately replay the input signal. If you understand the underlying physics, then you may understand what type of kernel functions are needed for sparse decomposition (for accurate but sparse representation of the input signal). For additional information, refer to (PoggioGirosi 1998).

Most of the physics-based modeling approaches considered involve theoretical physical chemistry. Specific, physics-based modeling approaches considered include:
1. Thermo-physical or thermo-chemical equilibrium calculations (LlnlCheetah 2021), (NasaGlennCEA 2021).
2. Molecular dynamics (ShanThompson 2013), (vanDuinDasgupta 2001).
3. First-principle (quantum mechanical) calculations of bond energies (GuoGuo 2018), (QuantumChemistry SW 2021).
4. Group additivity methods (HenryCurran 2019), (WikipediaGroupAdditivity 2021).

Similarities between physics-based modeling for energetic materials and alloys, per (SteingrimssonFanKulkarni 2020) and (SteingrimssonFanKulkarni 2021), are worth noting. Ref (SteingrimssonFanKulkarni 2020) and (SteingrimssonFanKulkarni 2021) describe use of machine learning in conjunction with thermodynamics, first-principle (quantum mechanical) calculations and molecular dynamics for the purpose of accelerating alloy design.

### 1. Calculations of Thermo-Physical or Thermo-Chemical Properties of Energetic Materials

The Cheetah software from LLNL and the CEA software from NASA comprise primary packages for calculating the thermo-physical or thermo-chemical properties of energetic materials (LlnlCheetah 2021), (NasaGlennCEA 2021).

Cheetah is a physics- and chemistry-based thermochemical computer tool that can predict the performance of ideal and non-ideal high explosives and explosive formulations. Cheetah is a premier energetic materials code addressing explosives, pyrotechnics and propellants. It is also an important formulation tool (LlnlCheetah9.0 2021). Cheetah harvests advanced concepts and theories of fluids and solids at high pressures and temperatures for purpose of modeling the thermodynamics of explosion products that result from the detonation of modern condensed, energy-dense explosives. By using models associated with the Chapman-Jouguet theory of detonation, Cheetah is capable of accurately calculating fundamental high-explosive features, such as detonation velocity, Chapman-Jouguet pressure, mechanical energy and more (LlnlCheetah9.0 2021). Cheetah possesses capabilities for doing rocket calculations, including the case of high total mass of propellant (high mₚ), calculations under various equilibrium conditions, frozen or unfrozen conditions, and for ionized gas or not. The Cheetah software can calculate both the specific impulse and detonation velocity. It also has capabilities that extend beyond high explosive calculations (LlnlCheetah9.0 2021). Pages 4-6 of (CheetahNextGen 2021) contain good, high-level description of the thermodynamic equations between product species that the Cheetah software solves to find chemical equilibrium.

The NASA CEA software calculates chemical equilibrium compositions and properties of complex mixtures. The CEA software is capable of computing assigned thermodynamic states, theoretical rocket performance, Chapman-Jouguet detonations, and shock-tube parameters for incident and reflected shocks. Associated with the CEA software are independent databases with transport and thermodynamic properties of individual species. Over 2,000 species are supported by the thermodynamic databases. Written in ANSI standard FORTRAN, the CEA software is in wide use by the aerodynamics and thermodynamics community, with over 2,000 copies in distribution (NasaGlennCEA 2021).

NPSS is a multi-disciplinary Numerical Propulsion System Simulation environment capable of modeling turbomachinery, air-breathing propulsion systems, liquid rocket engines, engine control systems and system model integration. NPSS is an advanced, object-oriented, non-linear thermodynamic modeling environment (SwriNpss 2021). REDTOP is a Rocket Engine Design Tool for Optimal Performance intended for use in the conceptual and preliminary design phases of space transportation systems utilizing liquid propulsion rocket engines (BradfordCharania 2004).

### 2. Simulation of Molecular Dynamics for Energetic Materials

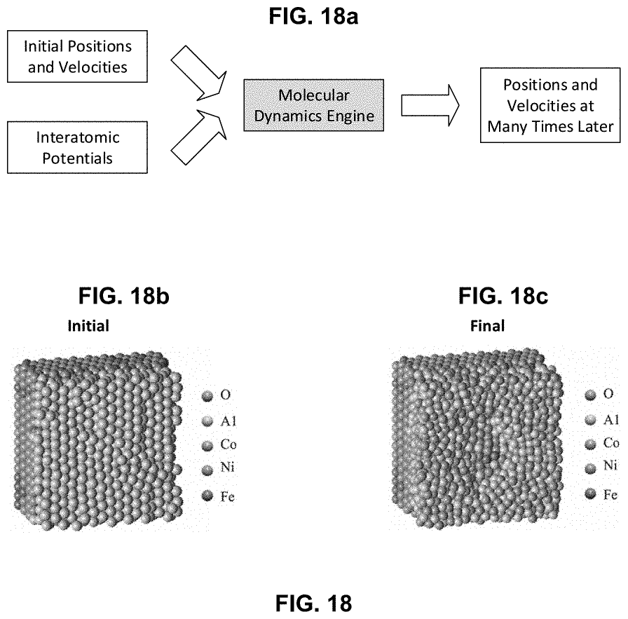

The Large-scale Atomic/Molecular Massively Parallel Simulator (LAMMPS) is a classical molecular dynamics simulator, with high-level structure shown in FIG. 18, aimed at materials modeling. More specifically, LAMMPS represents a collection of programs most often used for molecular dynamic simulations. LAMMPS supports potentials for solid-state materials, such as metals or semiconductors, and soft matters, such as biomolecules or polymers and coarse-grained or mesoscopic systems. It can be used to model an ensample of atoms or, more generically, as a parallel particle simulator at the atomic, meso or continuum scale. The programs comprising LAM MPS can be used to calculate diffusion and rearrangement of materials. These tend to be very microscopic, short time information about flame propagation, etc.

As most classical molecular dynamics simulators, LAMMPS requires the initial positions of the particles as input, LAMMPS can automatically generate initial positions for simple crystals or accept data files provided by user for materials with complex atomic structures. When provided with initial positions and velocities of the particles together with corresponding interatomic potentials, LAMMPS can simulate a wide variety of materials. While simulating a given material, LAMMPS can utilize statistical mechanics to convert atomic trajectories into macroscopic properties such as temperature, volume, pressure and density. LAMMPS can allow the user to specify which properties are desired as outputs. Statistical mechanics equations built into LAM MPS allow for the output of a variety of macroscopic properties. LAM MPS provides the tools to calculate the elastic constants of materials both at 0 K and at finite temperatures. The elastic constants are calculated by deforming the simulation box in six directions and measuring the resulting changes to the stress tensor. LAMMPS can determine the elastic constants of simulated materials at a variety of temperatures. Properties such as the bulk modulus, shear modulus, Young's modulus and Poisson ratio can be determined from the elastic constants. Accuracy of the simulated properties depends largely on the quality of the atomic potential input file. Determining the elastic constants through LAMMPS can give access to a variety of mechanical properties. Most materials with obtainable crystal structure and interatomic potential file can be simulated. Through simulation, important thermodynamic, structural and mechanical properties of the materials can be estimated. LAMMPS can run on single processors or in parallel using message-passing techniques and a spatial-decomposition of the simulation domain.

ReaxFF, a bond-ordered reactive force field, captures a practical approach to molecular dynamics simulations of large-scale, such as 1000s of atoms, reactive chemical systems (vanDuinDasgupta 2001). Classical MD uses classical force fields using pair interactions between atoms or molecules. These are generally non-reactive, so the bonds can vibrate and stretch but not break. Reactive force fields support making and breaking of bonds and therefore reactions. To this effect, traditional force fields are unable to model chemical reactions because of the requirement of breaking and forming bonds (a force field's functional form depends on having all bonds defined explicitly). ReaxFF, however, utilizes a general relationship between bond distance and bond order, on one hand, and between bond order and bond energy, on the other hand, that leads to proper dissociation of bonds to separated atoms. Other valence terms present in the force field, such as angle and torsion, are defined in terms of the same bond orders, and all these terms go gradually to zero during bond breakage. In other words, ReaxFF eschews explicit bonds in favor of bond orders, which allows for continuous bond formation/breaking. ReaxFF supports a class of potentials. It accounts for Coulomb and van der Waals potentials, for purpose of describing nonbond interactions between all atoms, with no exclusions (vanDuinDasgupta 2001). ReaxFF seeks to be as general as possible and has been parameterized and tested for hydrocarbon reactions, alkoxysilane gelation, transition-metal-catalyzed nanotube formation, and many advanced material applications such as Li ion batteries, TiO₂, polymers, and high-energy materials (WikipediaReaxFF 2021). MEAM, ADP, COMB and EAM comprise other classes of potentials used for molecular dynamics simulations (NISTpotentialRepository 2021).

For additional information on software applications involving Monte Carlo molecular modeling, molecular mechanics modeling, molecular design or Car-Parrinello molecular dynamics, refer to (QuantumChemistrySW 2021).

### 3. First-Principle (Quantum Mechanical) Calculations

It has been reported that, in the case of bipropellants, the relationship between the specific impulse and ignition delay time depends heavily on the electronic structures of the fuel involved (Vaghjiani 2021). This serves as a motivation to study the fundamental electronic properties of the fuel molecules involved. Longer bond lengths suggest weaker interactions between the atoms in a molecule. By collecting information on bond lengths of fuel molecules, and analyzing, one may identify the bond that will be the easiest to break (and hence serve as a likely trigger point for detonation).

Investigations of energetic materials from first-principle perspective involve computational modeling at atomic scale, for example of bond energies or phase stability. First-principle studies leverage calculations of quantum mechanics, with DFT approximating the Schrodinger equation, to simulate the electronic properties and stability of material candidates, from which the most promising candidates can be confirmed experimentally. VASP is software for atomic scale materials modelling, e.g. electronic structure calculations and quantum-mechanical molecular dynamics, from first principles. VASP is a tool to preform DFT calculations. VASP computes an approximate solution to the many-body Schrodinger equation, either within DFT, by solving a Kohn-Sham equations, or within a Hartree-Fock (HF) approximation, by solving Roothaan equations (SteingrimssonFanKulkarni 2020) and (SteingrimssonFanKulkarni 2021). NWChem is another software package for computational chemistry incorporating first-principle (quantum chemical) calculations. NWChem provides capabilities related to molecular mechanics, molecular dynamics, Hartree-Fock (self-consistent field method), density functional theory, time-dependent density functional theory as well as post-Hartree-Fock methods (WikipediaNWChem 2021).

For further exposition of first-principle (quantum mechanical) calculations applied to the design of alloys, refer to (SteingrimssonFanKulkarni 2020) and (SteingrimssonFanKulkarni 2021). The mechanism behind fracture of an alloy involves breakage of bonds between atoms, and hence exhibits some similarity with decomposition (detonation) of fuel molecules. Dislocations in case of alloys are defined in terms of discontinuities in atom bonding.

DFT can be used to calculate bond lengths and angles and activation barrier for bond breaking and chemical kinetics. It is worth noting that these DFT calculations tend to be computationally expensive, often taking hours to days for a single molecular structure. More accurate results are often associated with lengthier calculations performed at higher levels of theory. On the other hand, properly trained neural networks can (in theory) yield highly accurate predictions with relatively low computational cost, as noted in Table 3. First-principle (DFT) calculations can be used to validate (sanity check) the prediction outcomes of traditional ML systems, i.e., as a part of a hybrid computational system, such as presented in FIG. 11.

First-principle calculations can also be used in conjunction with molecular dynamics to study how specific atomic types and molecular structures control the detonating properties and performance of energetic materials. The ReaxFF reactive molecular dynamics can be combined with quantum mechanics molecular dynamics, in a so called R×MD(cQM) procedure, to predict the thermodynamic parameters of the Chapman-Jouguet state, as a measure of detonation performance (GuoGuo 2018).

Software applications involving first-principle calculations, density functional theory and quantum chemistry include Gaussian, Spartan, Jaguar and Molpro. For information on additional software applications, refer to (QuantumChemistry SW 2021).

### 4. Group Additivity Methods (in Context with First-Principle Calculations)

Within certain limits of accuracy the thermodynamic properties of properly selected atom groups within a molecule may add up to that of the molecule (WikipediaGroupAdditivity 2021). The group additivity methods pioneered by Prof. Sidney W. Benson uses the experimentally calculated heat of formation for individual groups of atoms to calculate the entire heat of formation for a molecule under study, possibly with some correction terms. The molecule is split up into groups, and each group makes a contribution to the thermodynamic functions of the overall molecule. Benson group-increment theory provides a quick and convenient way to estimate theoretical heats of formation for a new molecule without conducting time consuming experiments (WikipediaGroupAdditivity 2021), (HeatOfFormationAdditivity 2021).

The group additivity methods can be applied to most compounds, including alkanes, alkenes, alkynes, alcohols, ethers, ketones, aldehydes, cycloalkanes and aromatics. But parametrization against experiment and/or quantum chemical calculations is necessary for best results and widest range of applicability (HenryCurran 2019).

### 1 Example: Ethane

Ethane is a simple molecule with two identical groups (CH₃) and well known thermodynamics. Table 4 lists the contributions of each group to the enthalpy of formation, ΔH_{f}, and the specific heat capacity, Cₚ. Here, no correction term is needed. But the entropy of formation, ΔS_{f}, is obtained as the sum of the intrinsic entropies, ΔSᵢ₁ and ΔSᵢ₂, plus a correction term for symmetry, δS (HenryCurran 2019):

ΔS_{f}=ΔSᵢ₁+ΔSᵢ₁+δS (37)

where

δS=−R ln(σ). (38)

Here, σ represents the number of symmetries in the molecule (HenryCurran 2019):

σ=σₑₓₜ×σᵢₙₜ. (39)
σₑₓₜ represents the number of external symmetries (rotation of molecule), and σᵢₙₜ the number of internal symmetries (rotations around single bonds). In case of ethane, σₑₓₜ=2 and σᵢₙₜ=9, so

δS=−R ln(18). (40)

Similar corrections for optical isomers (OI) is +R ln(# of OI).

TABLE 4
Group additivity method applied to the two subgroups of ethane. The values for the enthalpy
of formation, ΔH_{f}, the entropy of formation, ΔS_{f}, and the specific heat capacity, Cₚ, can be obtained
from theNIST database (NISTdatabase 2021). The example has been adapted from (HenryCurran 2019).

| | Number of | ΔH_{f} (298 K) | ΔS_{f} (298 K) | Cₚ (300 K) |
|---|---|---|---|---|
| Group | Groups | [kJ/mol] | [J/mol/K] | [cal/mol/K] |
| C/H₃ | 2 | −42 | 102.62 | 26.25 |
| Symmetry Correction | | N/A | -R ln(18) | N/A |
| Total | | −84 | −229.28 | 52.49 |

### 2 Example: 2,2,4-trimethylpentane (iso-octane)

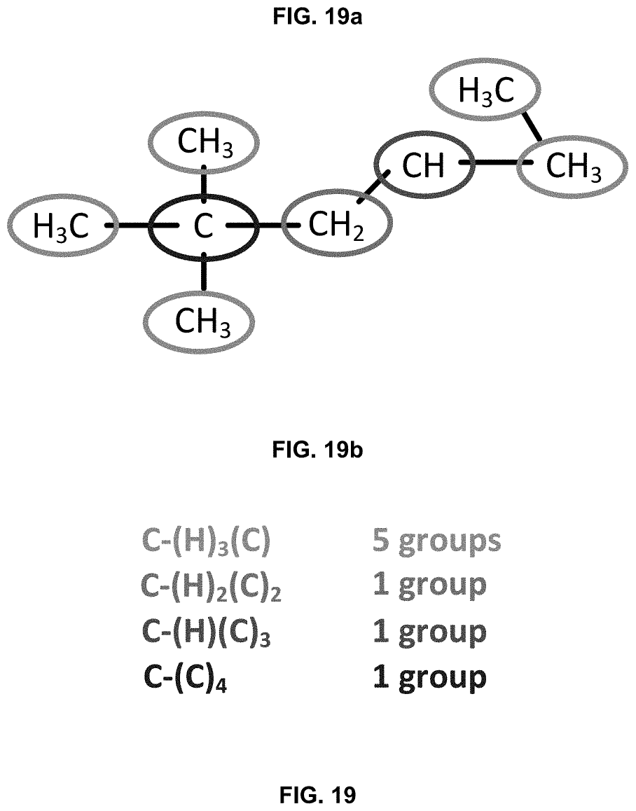

The subgroups comprising the 2,2,4-trimethylpentane (iso-octane) molecule are depicted in FIG. 19. Table 4 lists the contributions of each group to the enthalpy of formation, ΔH_{f}, the specific heat capacity, G, and the entropy of formation, ΔS_{f}, along with the correction terms. In case of the iso-octane molecule, σₑₓₜ=1 and σᵢₙₜ=729, so the symmetry correction becomes (HenryCurran 2019):

δS=R ln(729)=−13.1 cal/mol/K. (41)

A gauche interaction is a steric interaction that results between two groups on the adjacent carbon atoms in a Newman projection. The interaction occurs between two atoms or groups whose dihedral angle is more than 0° but less than 120°. A correction for each gauche interaction is introduced (0.7 kcal/mol). The number of gauche interactions or each non-terminal C—C bond in its most stable conformation is counted and summed (WikipediaGaucheEffect 2021).

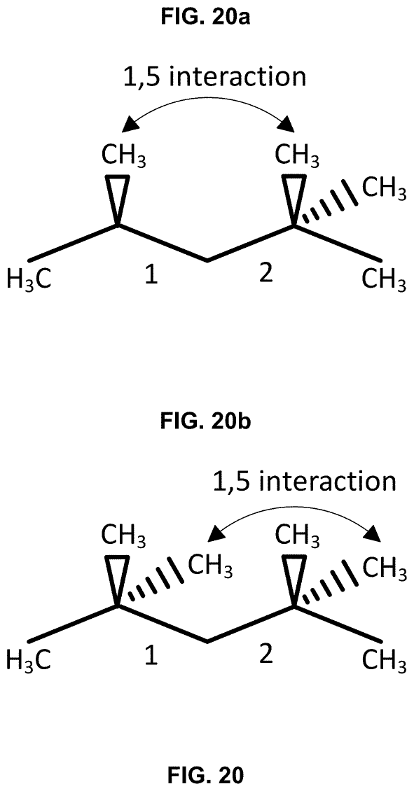

A 1,5 pentane interference correction, depicted in FIG. 20, is a steric hindrance that two terminal methyl groups experience in one of the chemical conformations of n-pentane (HeatOfFormationAdditivity 2021), (PentaneInterference 2021). A correction for the 1,5 pentane interaction is introduced (1.5 kcal/mol).

TABLE 5
Group additivity method applied to the subgroups of the iso-octane
molecule. The example has been adapted from (HenryCurran 2019).

| | Number of | ΔH_{f} (298 K) | ΔS_{f} (298 K) | Cₚ (300 K) |
|---|---|---|---|---|
| Group | Groups | [kJ/mol] | [J/mol/K] | [cal/mol/K] |
| C—(H)₃ (C) | 5 | −10.03 | 30.42 | 6.26 |
| C—(H)₂ (C)₂ | 1 | −4.96 | 9.42 | 5.44 |
| C—(H) (C)₃ | 1 | −2.13 | −12.02 | 4.39 |
| C—(C)₄ | 1 | 0.10 | −34.99 | 4.01 |
| Gauche | 3 | 0.70 | 0 | 0 |
| Interactions | | | | |
| 1,5 Pentane | 1 | 1.5 | 0 | 0 |
| Interaction | | | | |
| Symmetry | | | -R ln(729) | |
| Total | | −53.54 | 101.41 | 45.14 |

Software applications implementing Benson's group additivity methods include THERM (RitterBozzelli 1991), the NIST database program by Stein et al. (NISTwebBook 2021), THERGAS (MullerMichel 1995) and CRANIUM (MolecularKnowledge 2021). In case of THERM, the decomposition into groups and corrections is done by the user. CRANIUM supports groups of Joback as well as estimation of other properties, such as T_{eb}, T_{fus}, T_{c} and P_{c}.

### 7.3 From Macro-Scale to Micro-Scale (Molecular Level) Optimization

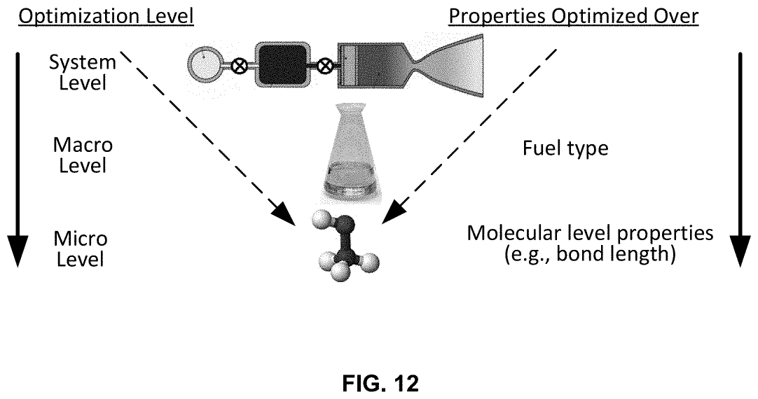

FIG. 12 visualizes an approach, where fuel properties are optimized at macro-scale, prior to optimization at micro-scale. We refer to macro-scale optimization as identification of properties, such as fuel type, oxidizer type or fuel-to-oxidizer ratio that yield the specific impulse and temperature of combustion. The macro-scale optimization addresses properties, including ignitability, that do not involve molecular-level properties directly. We similarly refer to micro-scale optimization as optimization that explicitly involves properties at the molecular level. These are properties, such as bond lengths, bond order, bond energies, bond dissociation energies, bond overlap population, substituent or functional groups.

For purpose of systematically closing in on desired properties, it may make sense to roughly compare properties at the macro level, prior to detailed optimization at the molecular level. The macro-level optimization can inform a designer of the ranges of specific impulse to expect for given propellants (or propellant types).

The heat of combustion entails one linkage between the macro-scale and micro-scale optimization. The heat of combustion is a macroscopic property. But it is affected by the presence of functional groups, such as azido, amino or nitro groups, i.e., by microscopic properties.

The fuel ignitability, and associated trigger points, entail another linkage between the macro-scale and micro-scale optimization. While a propellant is not expected to ignite upon the slightest change in environmental conditions, it is expected to be ignitable. The database outlined in this invention accounts for microscopic properties, such as trigger points for ignition, which are related to the bond dissociation energy and bond lengths. The nitro group (R—NOO) has been found to be the most likely trigger point in many compounds.

### 7.4 Specific Approaches to Prediction Incorporating Joint Optimization Energetic Materials

### 1. Introduction

The methods presented here constitute an enhancement over the joint optimization for alloys introduced in (SteingrimssonFanKulkarni 2020) and (SteingrimssonFanKulkarni 2021).

Selection of energetic materials is usually based on many factors, as noted above. A good propellant needs to have high specific impulse, relatively high density, high combustion temperature as well as low molecular weight of the gaseous products. Key trade-offs involve

1. Detonation performance of the HDEM vs. its thermal stability;
2. Specific impulse of the HDEM vs. its mass density.

### 2. Approach Motivated by Analytical, Physics-Based Relations

Motivated by (SteingrimssonFanYang 2021), we seek to formulate prediction models that effectively capture the underlying physics.

In the case of the trade-off between the detonation performance and the thermal stability, a minimal, but representative, optimization problem can be formulated as follows:

max.[ISP(specific impulse),SADT(self-accelerating decomposition temperature)]

subject to

ρ<C₁;

T_{c}>C₂;

Volume<C₃.

The constraints here capture design requirements related to low system weight and high combustion temperature. Carrying large mass of fuel consumes energy. Therefore, we list an upper limit on the mass density as the first constraint. But the rockets also possess finite containers (volume) for storing the fuel. For that reason, the volume constraint may translate into a lower limit on the mass density. Additional constraints, e.g., safety constraints, may be similarly introduced to capture other design requirements.

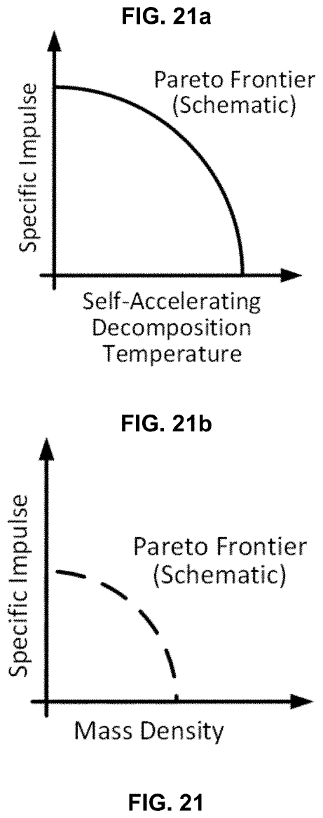

The solution to this problem may not consist of a single solution, but a set of solutions that form a Pareto frontier. FIG. 21a illustrates one such Pareto frontier. A Pareto frontier represents the set of all Pareto efficient (jointly optimal) allocations. A Pareto efficient, also referred to as a Pareto optimal, situation is a condition, where no individual or preference criterion can be better off without making at least one individual or preference criterion worse off or without any loss thereof (WikipediaPareto 2021).

But in the case of the trade-off between the specific impulse and the mass density, a representative optimization problem can be formulated as follows:

max.[ISP(specific impulse),ρ(mass density)]

subject to

T_{c}>C₄;

SADT<C₅;

Volume<C₆.

The constraints here capture design requirements related to low system weight and good thermal stability. Additional constraints may be similarly introduced to capture other design requirements.

Again, the solution to this problem may not consist of a single solution, but a set of solutions that form a Pareto frontier, such as the one presented in FIG. 21b. High fuel density is desired in order to accommodate a large amount of propellant in a given volume on a spacecraft, as noted above. But no single fuel can both provide both high specific impulse and high density. In practice, as the fuel density decreases, the specific impulse increases (DouglasRapp 1990).

### 3. Approach Motivated by Stochastic Processes

This approach is an extension of the computational techniques presented in (WangGongLiuDu 2011). Ref. (WangGongLiuDu 2011) presents semi-empirical relations for the purpose of identifying HEDCs applicable for propellant among the derivatives of DPO with —N₂, —ONO₂, and NNO₂ groups. Here we grow the set of propellants supported beyond the derivatives of DP (2,5-dipicryl-1,3,4-oxadiazol e) with N₃, —ONO₂, and NNO₂ groups outlined in (WangGongLiuDu 2011).

In the case of explosives with CHNO elements, the detonation velocity and pressure can be calculated using the Kamlet-Jacobs equations (WangGongLiuDu 2011). Here, consistent with Eq. (18) and Eq. (19),

D=(1.011+1.312ρ₀)(NM̅^{0.5}Q^{0.4})^{0.5} (42)

P=1.558 ρ₀²NM̅^{0.5}Q^{0.5} (43)

As before, P represents the detonation pressure (GPa), D is the detonation velocity (km/s), ρ₀ is the packed density (g/cm³), Nis the moles of gas produced per gram of explosives, M is an average molar weight of detonation products, and Q is the chemical energy of detonation (kJ/g).

In case of the nitrate derivatives of DPO (II-1 to 11-5), with the number of nitrate (n₂=0, 1, 2, 3, 4) groups, OB₁₀₀, ΔH_{f}, ΔH_{comb}, Vₘ, ρ, D and P exhibit linear dependence on n₂ (WangGongLiuDu 2011). Here, the relationship between OB₁₀₀, ΔH_{f}, ΔH_{comb}, Vₘ, ρ, D, P and the number of nitride group (n₂) can be presented in the form of semi-empirical linear relations as follows:

OB₁₀₀=4.81−0.27n₂; (44)

ΔH_{f}=265.06−102.74n₂; (45)

ΔH_{comb}=1423.18+33.37n₂; (46)

Vₘ=262.52+25.87n₂; (47)

ρ=1.88+0.03n₂; (48)

D=7.92+0.30n₂; (49)

P=28.47+2.67n₂. (50)

For the nitramine derivatives of DPO (III-1 to III-5), ΔH_{f}, ΔH_{comb}, Vₘ, r, D and P, except OB₁₀₀ that linearly decreases, increase with the number of nitamine (n₃=0, 1, 2, 3, 4) groups. Their linear relationships can be specified as (WangGongLiuDu 2011):

OB₁₀₀=4.78−0.40n₃; (51)

ΔH_{f}=287.74+70.51n₃; (52)

ΔH_{comb}=1432.32+35.15n₃; (53)

Vₘ=264.48+25.98n₃; (54)

ρ=1.87+0.03n₃; (55)

D=7.88+0.30n₃; (56)

P=28.05+2.62n₃. (57)

We have established here semi-empirical relations between the outputs (D and P) and the corresponding inputs (n₂ and n₃).

This invention draws upon a structurally similar approach, outlined in (SteingrimssonFanKulkarni 2020) and (SteingrimssonFanKulkarni 2021), for predicting low-cycle fatigue (SN curves) for alloys. Upon replacing the deterministic coefficients in Eq. (42)-(50) with random variables, one obtains the random process described:

D=(r₁+r₂ρ₀)(NM̅^{0.5}Q^{0.5})^{0.5}; (58)

P=r₃ρ₀²NM̅^{0.5}Q^{0.5}; (59)

OB₁₀₀=r₄+r₅n; (60)

HOF=r₆+r₇n; (61)

Q=r₈+r₉n; (62)

V=r₁₀+r₁₁n; (63)

ρ=r₁₂+r₁₃n; (64)

D=r₁₄+r₁₅n; (65)

P=r₁₆+r₁₇n. (66)

For this illustrative example, our invention assumes carrying out regression on the on the coefficients r₁-r₁₇ (SteingrimssonFanKulkarni 2020) and (SteingrimssonFanKulkarni 2021). This allows one to broaden the set of compounds covered by the approach. In case of multi-mode propulsion, one can initially optimize over the different modes (e.g., over a chemical part and an electrical part) separately, and then combine. Other techniques, such as neural networks, decision trees, decision tables, support vector machines, Bayesian networks, genetic algorithms, reduced error pruning trees or M5 model trees, can also be applied for estimating unknown coefficients in a realization of a stochastic process. But in this case, the number of unknown coefficients (r₁-r₁₇) is small enough that regression analysis may be considered a suitable technique. The suitability depends in part on the number of unknown parameters in the technique employed relative to the number of data points available to estimate the model implemented by the technique. For further specifics, refer to (SteingrimssonFanKulkarni 2020), (SteingrimssonFanYang 2021) and (SteingrimssonFanKulkarni 2021).

In summary, the approach consists of the following steps:
1. Conducting regression analysis on the random variables, to estimate corresponding coefficients, for known compounds.
2. Predicting corresponding coefficients for unknown compounds, using the regression model.
3. Plugging corresponding coefficients for unknown compounds into the model (Eq. (42) (43)) to estimate output properties of interest.

### 4. Approach Motivated by Bayesian Bootstrapping

The Bayesian bootstrapping approach combines experimental data with computational predictions, to rapidly, reliably and robustly identify the compositions of energetic materials that are most likely to meet a multi-criterion design specification, such as the one listed in Table 6. The approach predicts both the expected value and the associated uncertainty in the individual properties of energetic materials. The approach evaluates the likelihood that a candidate energetic material will satisfy the design criteria specified. It then selects the candidates most likely to fulfill these design criteria.

The Bayesian bootstrapping approach, motivated by (ConduitJones 2017), but here applied to energetic materials, consists of two key steps:
1. In the first step, neural network models are trained separately for individual properties of energetic materials.
2. In the second step, Bayesian inference (a Bayesian bootstrapping approach) is employed for maximizing the probably that each of the input properties exceeds a target value (a design criteria) specified.

The second step involves joint optimization of the input properties. This step (the Bayesian inference) reflects a primary strength of the bootstrapping approach. The approach is capable of accounting for the properties, that can be predicted computationally, and combine with experimental values for the properties, that cannot be predicted computationally. But on the other hand, the

TABLE 6
Approaches used by the Bayesian bootstrapping to predict individual,
representative properties of energetic materials. The property
descriptor physical refers to the fact that the property
in question can be measured, without specifying how
it is measured. Additional properties involving
molecular dynamics may be obtained using LAMMPS.

| Property | Approach | Target | |
|---|---|---|---|
| Fuel cost | Physical | $ 0.20/kg | |
| Density | Physical | 1.3 | g/cc |
| Specific impulse (shifting) | Neural net | 280 | sec |
| Specific impulse (frozen) | Neural net | 275 | sec |
| Oxidizer-to-fuel ratio | Neural net | 7.0 | |
| Combustion temperature | Physical | 2990K | |
| Ratio of specific heats | Physical | 1.2 | |
| Oxidizer density | Physical | 1.450 | g/cc |
| Oxidizer freezing point | Physical | −1° C. | |
| Oxidizer boiling point | Physical | 150° C. | |
| Fuel density | Physical | 0.81 | g/cc |

| | | |
|---|---|---|
| Fuel freezing point | Physical | −73° C. |
| Fuel boiling point | Physical | 147° C. |

approach may rely on significant amount of experimental input data.

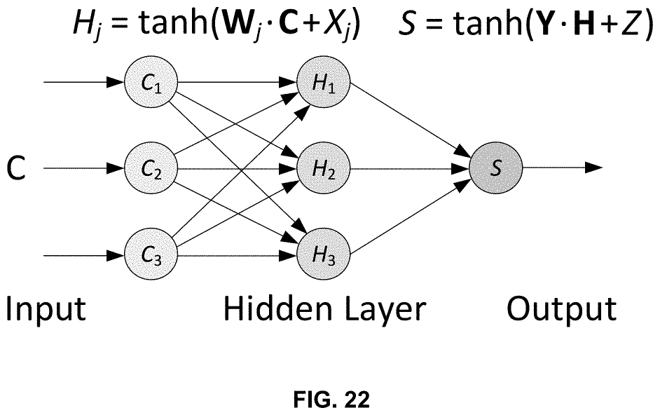

In the first step, a neural network model is constructed that predicts the physical properties S(C), for a candidate energetic material, C. The structure of the neural network is presented in FIG. 22. The network accepts three input variables, C=[C₁, C₂, C₃]. The output of the hidden layer is the indicator function

Hⱼ=tanh(Wⱼ·C+Xⱼ);j=1,2 or 3. (67)

The hidden nodes Hⱼ are combined again by the indicator function to provide the final output S:

S=tanh(Y·H+Z). (68)

The undetermined coefficients in this model are the weighting coefficients for the hidden layer, W₁₁, W₁₂, W₁₃, W₂₁, W₂₂, W₂₃, W₃₁, W₃₂ and W₃₃, the bias coefficients for the hidden layer, X₁, X₂, and X₃, the weighting coefficients for the output layer, Y₁, Y₂ and Y₃, and the corresponding bias, Z. The undetermined coefficients are optimized by minimizing a reduced chi-squared statistic over the preexisting data:

$${χ}_{red}^{2} = \frac{1}{N - ( 2 + {N}_{D} ) {N}_{H} - 1} {{\sum }_{j = 1}}^{N} \frac{{( {S}_{j} - {s}_{j} )}^{2}}{{σ}_{j}^{2}} \qquad ( 69 )$$

Here, N represents the number of data points of values {s} and experimental uncertainty {σ} that are available for training, N_{H} denotes the number of hidden nodes and ND the number of input parameters.

Whereas a neural network model may be considered suitable in this case, other techniques, such as multi-variate regression, decision trees, decision tables, support vector machines, Bayesian networks, genetic algorithms, reduced error pruning trees or M5 model trees can also be used to predict individual properties of energetic materials.

In the second step, a separate system of neural networks, with a separate set of undetermined coefficients to be optimized, is constructed for each output Sᵢ. To evaluate uncertainty in the predictions, a committee of M neural network models is constructed, labeled j∈{1, 2, . . . , M}, using Bayesian bootstrapping, that yields predictions S(C)ⱼ. Each neural network model is assembled by associating random weight with the input data, which deliver a range of outputs correctly distributed to reflect the underlying uncertainty in the networks due to limited input data. For the energy material candidates proposed, the average value over the models gives the predicted physical value

$${V}_{C} = \frac{1}{M} {\sum }_{j = 1}^{M} {S}_{C , j} . \qquad ( 70 )$$

The corresponding uncertainty is determined through the covariance matrix

$${\sum }_{C} = {\sum }_{j = 1}^{M} ( {S}_{C , j} - {V}_{C} ) {( {S}_{C , j} - {V}_{C} )}^{T} . \qquad ( 71 )$$

Knowledge of the uncertainty is vital, as it allows the designer to balance the risk of materials with lower uncertainty, but that are less capable, with more promising, but also more speculative, energy material candidates.

### 5. Other Approaches Employing Physics-Based Modeling

Ref (RettigRitterHelmer 2015) describes a scheme for joint optimization of the mechanical properties of Ni-based superalloys. The scheme utilizes an interface to CALPHAD to estimate properties for synthetic candidates. In this way, limited experimental data can be augmented. The fundamental ideas can be extended both to high-entropy alloys and energetic materials, as further addressed in the next section. For example, hybrid approaches, along the lines of FIG. 11 or FIG. 13, can be formulated utilizing an interface to physics-based modeling tools incorporating molecular dynamics or first-principle calculations.

### 7.2 Continuation in Part: Additional Approaches to Joint Optimization of Alloy Properties

This part of the invention describes tools to assist with design of alloys exhibiting exceptional performance, e.g., at high temperature. Such tools can enable development of materials capable of withstanding the high temperatures in a turbine, as well as the extreme stresses and the harsh service conditions, that turbine blades are subjected to.

### 1. Background Motivation

Gas turbines are widely used for electric power generation and aircraft propulsion. Turbines currently provide ˜97% of the US electric power and are used in almost all power plants. Presently, natural gas fueled turbines produce approx. 35% of the total electricity production in the US. Further, air travel is responsible for ˜2% of carbon emissions. This is anticipated to double in the next two decades. Improving efficiency of industrial gas turbines is, thus, a very important issue for energy savings, carbon emissions, and the economy of those and related industries. Since turbines make up for nearly all of the world's electric generation capacity, incremental efficiency improvements can have very large impacts on the bottom line of energy production, and on fuel consumption in general, and can result in huge reduction in the carbon dioxide emitted.

Assuming fossil-fuel combustion, the first stage of a modern turbine (the stage directly following the combustor) typically faces temperatures around 2,500° F. (1,370° C.). Modern military jet engines, like the Snecma M88, can experience turbine temperatures of 2,900° F. (1,590° C.). Such high temperatures weaken the turbine blades and make them more susceptible to creep failures. The high temperatures can also make the blades susceptible to corrosion failures. In addition, vibrations from the turbine can cause fatigue failures.

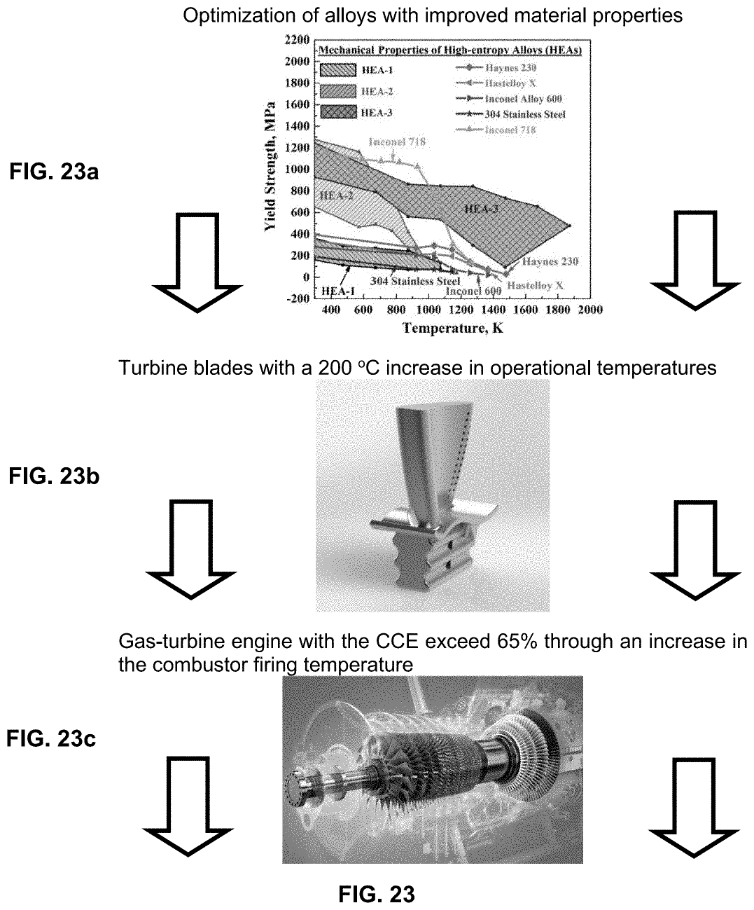

Increasing the operating temperature results in higher thermal to electric conversion efficiency, which results in better use of the fuel source, lower cost per kilowatt hour, and lower greenhouse emissions per unit of power generated, as noted above. Current stage-one blades made from nickel-based superalloys have limitations in material properties, which cap the combined cycle efficiency (CCE) of gas turbines to 62%. In order to achieve CCE in excess of 65%, a 300° C. increase in the combustor firing temperature is required. This calls for refractory high-entropy alloys (RHEAS) as stage-one turbine blade materials, capable of operating at 1,300° C. RHEAS are chosen in part on basis of their superior ability to retain strength at high temperatures, as shown in FIG. 23. The materials developed using the tools (joint optimization technology) presented have the potential of improving the theoretical efficiency of land-based gas turbines by 7-9%, using advanced alloy systems, such as RHEAs, in the turbine blades (SteingrimssonFanKulkami 2021).

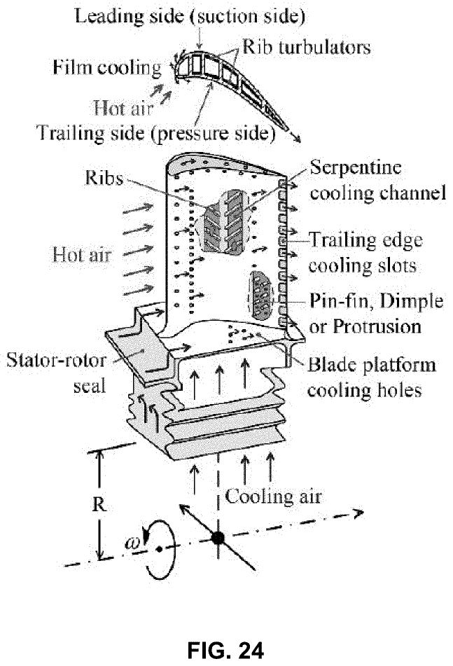

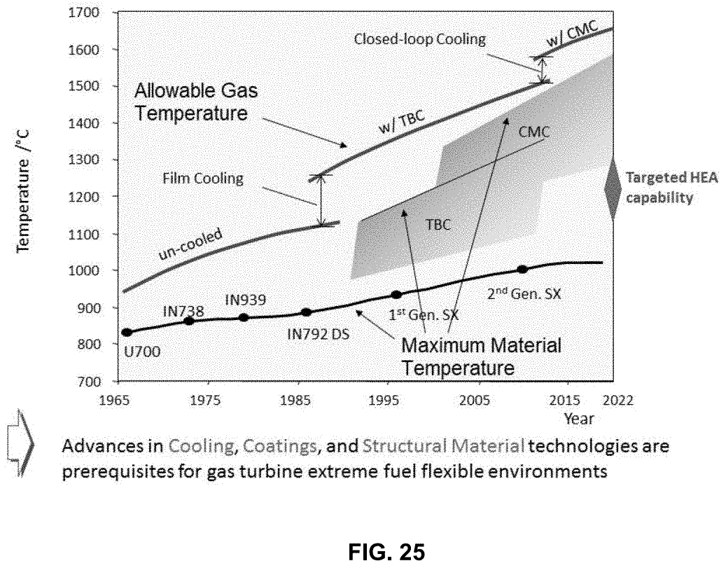

FIG. 25 shows an increase in the temperature capability of 150° C. for Nickel-based superalloys over 50 years. Nickel-based alloy turbine blades currently in production are provided with internal serpentine coolant passages. They are also provided with thermal barrier coatings (TBCs) working in conjunction with an extensive film cooling scheme, as presented in FIG. 24 and FIG. 25. At the component level, these alloys need to be produced with minimal extrinsic defects and be amenable to subsequent thermomechanical processing.

The integrated modeling tool presented contributes to the disruptive and transformative materials development needed for replacing current Ni-superalloys, employed for stage-one blades, with RHEAs. Integrated design and manufacturing of new ultra-high temperature materials for gas turbine blades may enable ˜7-9% increase in CCE, assuming a >200° C. increase in solvus temperature of the base alloy over the state of the art. This invention can result in successful design of high-performance refractory high-entropy alloys that will increase the temperature capability of the base metals to ≥1300° C. This is in sharp contrast to current state-of-the-art Ni-based superalloys used in turbines that cannot operate at T≥1100° C. without thermal barrier coatings.

The invention has the potential to result in reduction in fuel consumption for Siemens SGT-A05 aero-derivative gas turbine fleet by 71,505 ton/yr. A 7% improvement in efficiency in the natural gas turbines used for electricity generation (for civilian aircraft) represents saving up to 15-16 quads (3-4 quads) of energy (NewellHagerty 2020), (FederalAviation 2020), (BureauTransportation 2020).

### 2. Representative Process for Alloy Design A Closed-Loop System Design Approach

Table 7 and

Table 8 summarize representative requirements for refractory HEA material systems presented to an alloy designer. The requirements are presented in part relative to the state of the art (SOA), i.e., the Ni-based superalloys. In response to requirements from the customer, the alloy designer may outline performance improvements considered achievable, presented in Table 9. Requirements for design of a turbine blade are multi-facet in nature (call for a balance). Much of the past success of Ni-based superalloys can be attributed to their ability to strike proper (design) trade-offs.

TABLE 7
Basic threshold of mechanical properties for base alloys and coatings,
and manufacturability criteria (DE-FOA-0002337).

| Property | Qualifying/benchmark threshold | Comments |
|---|---|---|
| Creep strain at | <2% | Use relevant ASTM E139-11 test |
| 1300° C., 200 MPa, | | method, or equivalent. |
| 100 hours | | |
| Room temperature | >1.5% | N/A. Test just needs to be run to |
| (RT) tensile | | verify. |
| ductility | | |
| RT fracture | >10 MPa/m^{1/2} | Use relevant ASTM E399, |
| toughness | | E1820 or equivalent |

| | |
|---|---|
| Coating | Base alloy with coating must retain its |
| performance | ductility at RT after exposure to 1700° C. in air. |
| Manufacturability | Standard tensile mechanical test specimens are |
| | manufactured using the same process as would be |
| | used for manufacturing turbine blades and must meet |
| | critical dimensional requirements. |

TABLE 8
Comprehensive benchmark metrics of ultrahigh temperature alloys, coatings and manufacturability criteria
(DE-FOA-0002337).

| ID | Property | Benchmark | metrics Rationale |
|---|---|---|---|
| 1 | 0.2% tensile yield | ≥400 MPa | Current SOA is 450 MPa at 1,050° C. (Use |
| | strength at 1300° C. | | relevant ASTME21-17e1 test method) |
| 2 | Solidus temperature | ≥1,500° C. | Current SOA is 1,200-1,350° C. |
| 3 | Density at RT | ≤9.0 g/c.c. | Current SOA is ≤ 9.0 g/c.c. |
| 4 | Thermal conductivity | RT: 9-12 W/m · K | Typical Ni superalloys: |
| | | 1,300° C.: ≥W/m · K | RT: 11 W/m · K |
| | | | 800° C.: 22 W/m · K |
| | | | 1,250° C.: 23.5 W/m · K |
| | | | (Use relevant ASTM E1225-13 test |
| | | | method) |
| 5 | Linear thermal expansion | ≤2% | Current SOA is 2.2% (RT-1,200° C.); |
| | (RT-1300° C.) | | CTE: 8-18 × 10⁻⁶/° C. (Use relevant |
| | | | ASTM E228-17 test method) |
| 6 | Thermo-mechanical | 0.45% strain; R = −1; 100- | Current SOA is 0.45% strain; R = −1; |
| | fatigue (TMF) | 1,300° C.; ≥1,000 cycles | 100-950° C.; 1,000 cycles |
| 7 | Coating performance | Base alloy with coating must retain | See 7.2.2 of (DE-FOA-0002337) for |
| | | creep strength after exposure to | details for test methods and metrics |
| | | 1,700° C. in air. | |
| 8 | Manufacturability | Manufacture a generic small turbine | See 7.3.2 of (DE-FOA-0002337) for |
| | | blade as a demonstration | details of test methods and metrics |

In order to increase the combined cycle efficiency to 65%, a closed-loop system design approach is needed to procure novel alloys with the target properties specified. At a high level, three (3) primary hurdles face the use of RHEAS in critical, high-performance applications:

1. Identification of an optimized alloy from the vast number of possible compositions;
2. Ductilization, toughening and oxidation resistance; and
3. Processability and production feasibility.

To address challenges related to an exponentially large design (composition) space (SteingrimssonFanKulkarni 2021), it makes sense to design the candidate RHEAs using a concurrent approach involving iterations among modeling, characterization, processing, high-throughput (HTP) testing, and manufacturing. The large RHEA composition space presents challenge to optimization, in particular to maintain the required phase stability, thermo-mechanical, physical, environmental-related properties for operating in the extreme environments of the gas-turbine engines. But the exponentially large design space of RHEAS also provides unique opportunities to design (down-select) compositions and microstructures to deliver desired properties.

TABLE 9
Overview over the performance improvements considered achievable.

| Factor | Quantitative Metrics | Improvement |
|---|---|---|
| Thermophysical | a) Solvus temperature-1,550° C.-1,850° C. | a) 300° C. higher |
| properties | b) Density < 9 g/cc | b) Equivalent |
| | c) Thermal conductivity [Room-temperature-1,300° C.]-10-14 | c) Equivalent |
| | W · m K | d) Equivalent |
| | d) Coeff. of thermal expansion (RT-1,300° C.)-8-18/° C. | |
| Mechanical | a) Yield strength @1,300° C. ≥ 400 MPa; RT tensile ductility > | All properties- |
| properties | 1.5% | Equivalent |
| | b) RT fracture toughness > 10 MPam^{1/2} | value & higher |
| | c) Thermo-mechanical fatigue (0.45% strain; Strain ratio (R) = −1; | temperature |
| | 100-1,300° C.) ≥ 1,000 cycles | |
| | d) Creep strain at 1,300° C., 200 MPa, 100 hours < 2% | |
| Coating | Coating must retain creep strength after exposure to 1,700° C. in air | Equivalent |
| performance | | value @ higher |
| | | temperature |
| TBC | Bond coat oxidation and TBC spallation life @1,300° C.-Retain | Equivalent |
| compatibility | current temperature limits | value @ higher |
| | | temperature |
| Combined cycle | >65% target | ~3-5% higher |
| efficiency | | |
| Manufacturability/ | Processability with defect free structure and meet critical | Flexible |
| repairability | component geometry requirements | manufacturing |

TABLE 10
Sample properties of initial compositions for the design
of RHEAs for high-temperature turbine applications

| Composition (all | Predicted | Tₘ | Density | Strength | Poisson's |
|---|---|---|---|---|---|
| added ~1 at. % Re) | Phase | [° C.] | [g/cc] | [GPa] | Ratio |
| Cr_{0.5}V₂NbNi_{1.5}W_{0.5} | BCC + FCC | 1,721 | 9.0 | 2.9 | 0.33 |
| CrV₂NbTaTiZr | BCC | 1,991 | 8.6 | 2.4 | 0.33 |
| AlV₄NiTiW | BCC + B2 | 1,930 | 8.5 | 2.4 | 0.34 |
| AlCrMoNbTaTiV₂ | BCC | 2,100 | 8.9 | 2.0 | 0.33 |
| Al_{0.5}CrNiV₃W_{0.8} | BCC + B2 | 1,931 | 8.8 | 2.9 | 0.32 |

We recommend performing alloy design, microstructure control, processing, testing, and manufacturing within the closed-loop system design approach mentioned above. We, further, recommend developing the RHEAS through use of a system design approach (a down-selection approach) comprising of

### 1. Integrated Computational Materials Engineering (ICME),

2. HTP mechanical testing,
3. Processing and advanced manufacturing.

It makes sense for the ICME to iteratively integrate ML modeling of phase fields, Calculation of Phase Diagrams (CALPHAD), Density Functional Theory (DFT), also referred to as first-principle calculations, multiscale (atomic, meso-scale and continuum-scale) modeling and simulation, as well as joint ML optimization of material properties, to accelerate the materials development, processing, and testing cycle. The HTP mechanical testing can help down-select samples for evaluating mechanical resilience under long-term exposure in extreme environments. One can then refine the RHEA compositions to optimize the microstructures, fabricate coupons, and perform tensile experiments and oxidation testing to ultimately deliver RHEAs possessing the target properties. RHEA performance characteristics, that need to be experimentally verified, include thermal stability of the designed microstructures, mechanical strength, and fatigue-cycle endurance at high operational temperatures (defined as temperatures above 1,300° C.).

At more granular level, ICME portion of the system design approach mentioned above can consist of the following steps:
1. High-throughput ML prediction of phase field, to predict and validate several high-temperature, high-strength, tough and oxidation-resistant RHEA candidates.
2. Development and validation of CALPHAD thermodynamic databases for the down-selected RHEAs.
3. Multiscale simulations, to obtain fundamental understanding of defect structures and deformation mechanisms, as well as microstructure evolution in extreme environments.
4. Joint ML optimization of mechanical properties, for further refinement.

Regarding Step 1, refer to Table 10 for examples of the RHEA candidates mentioned. The high-throughput ML prediction can be employed to optimize the competing stage-one blade alloy requirements of high melting points, moderate densities, and high strengths while also having high toughness. High melting points 1,900° C. can ensure high yield strengths at 1,300° C. T₀ minimize oxidation, appropriate amounts of Al, Cr, Ni, Ti, Fe, Y, and Re may be added to the RHEAs. T₀ address the brittleness of BCC RHEAs, one can exploit composite hardening and have identified two RHEA composites with BCC+FCC and BCC+B2 phases, to initiate the process.

Regarding Step 2, one may also consider performing HTP calculations, simulate precipitation, and predict stability of HEA oxides. CALPHAD will be utilized to validate the phase field and microstructure stability proposed by the ML prediction.

Regarding Step 3, the multiscale simulations allow designers to verify the efficacy of alloy design and microstructural-strengthening mechanisms across length scales. Here, the designer can look to maximize elevated-temperature solution hardening as well as the oxidation resistance. The optimized alloys with stable microstructures at 1,300° C. will be evaluated using HTP testing and analyzed as a part of the multiscale simulation. Formation of hierarchical microstructures, if present, can be noted. Calculations of phase-equilibria, diffusion fields (minimization of vacancy diffusivity), precipitation, and multiscale structure and dynamics form an important foundation for designing the desired RHEAs.

In Step 4, one can strike attractive balance between material strength, ductility, toughness, creep strain, fatigue, fracture toughness, environmental (oxidation) resistance and conductivity. The joint ML optimization is further addressed below.

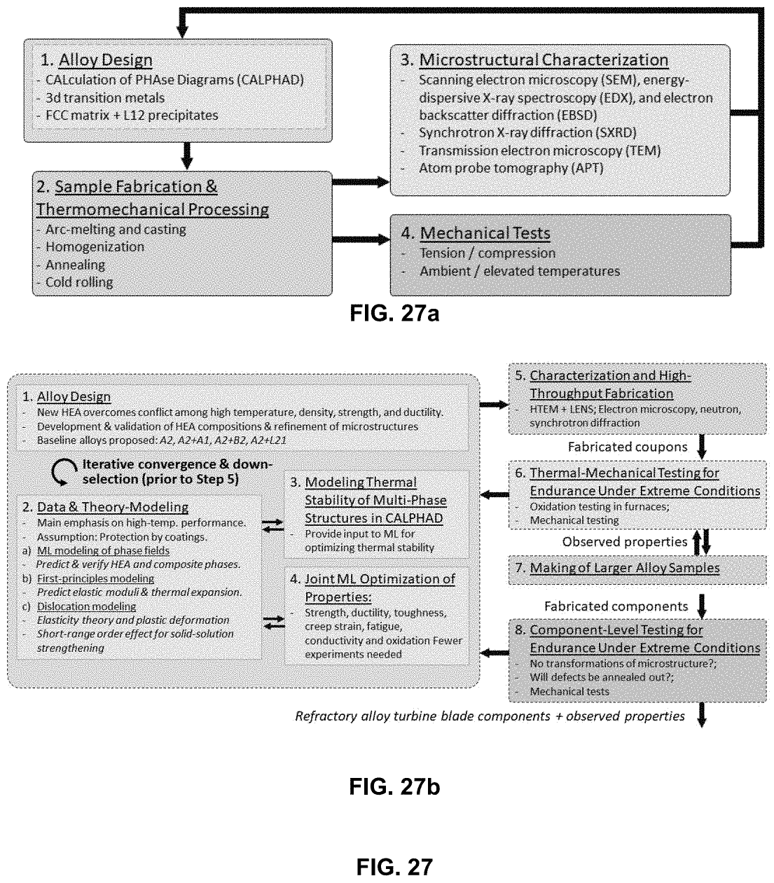

FIG. 27 presents a representative ICME process for the design of refractory HEAs suitable for extreme service environments, such as in a turbine. Table 11 shows the pros and cons of different methods for modeling the mechanical and physical properties of HEAs.

TABLE 11
Pros and cons of different methods for modeling the
mechanical and physical properties of HEAs.

| Approach | Strength | Weakness |
|---|---|---|
| Correlative Approach (ML) | Enables fast searches over large | Due to correlative nature, ML relies |
| | composition spaces | on quite a lot of input data for |
| | | accurate estimation |
| Multiscale Modeling | Able to handle many length scales; | Need quite a lot of results; Relies |
| | Physics-based | on assumptions; Cannot calculate |
| | | everything; Long computation time |
| CalPhad | Can simulate phase stability | Relies on empirical results (input |
| | | databases) |
| First-Principle | Precise | Cell for calculations is too small |
| Expert Insight | Partially captured in multiscale | Not computer accessible without |
| (Dislocation Modeling) | modeling: Accounts dislocations and | manual programming |
| | first-principle. | |

### 4. Extension of Bayesian Bootstrapping to High-Entropy Alloys

The approach of Conduit et al. (ConduitJones 2017) does not only predict the individual material properties (outputs) of interest, but also provides associated variance estimates. For a candidate data point far away from a data point in the input training set, chances are that the variance in the output predicted will be high. But it is theoretically possible for one to obtain a prediction with low variance in the output, even in case of a candidate data point far away from a data point in the input training set.

The approach of Conduit et al. (ConduitJones 2017) consists of two steps. In the first step, neural network models for individual physical properties S(C), for composition, C, are trained separately. These neural network models resemble FIG. 22. In the second step, a Bayesian inference (a Bayesian bootstrapping approach) is applied to maximize the probably that each of the input properties exceeds the target value listed. These target values resemble the ones listed in Table 6. The joint optimization is introduced in the second step.

While the approach of Conduit et al. (ConduitJones 2017) benefits from Bayesian inference, it may rely on significant amounts of input (largely experimental) data, for accurate prediction. Such data may have existed for Ni-based superalloys, but less so for the HEAs. As vigorous research on the HEAs continues, the availability of experimental data increases considerably by each year that passes by. A key undertaking entails characterization of the extent to which the experimental input data available for HEAs suffices, for purpose of this method. Here, one would compare and contrast the experimental data needed by the method, to attain accurate estimation, to the experimental data available.

### 5. Joint Optimization of Mechanical Properties of Alloys with Enhanced Fidelity Through Integration of Machine Learning with CALPHAD, DFT or Empirical Rules

The joint optimization schemes outlined here involves extension to RHEAS of techniques originally developed by Rettig et al. (RettigRitterHelmer 2015) and by Conduit et al. (ConduitJones 2017), both addressing (and proven for) the design of Ni-based superalloys. For a note on the availability of data for Ni-based superalloys vs. HEAs, refer to the previous paragraph.

### 5.1 Extension of (RettigRitterHelmer 2015) from Superalloys to HEAs Still CALPHAD Based

The approach of Rettig et al. (RettigRitterHelmer 2015) relies on the use of use CALPHAD to generate synthetic data points in the parameter space and project from there (feed the synthetic data points into a model for purpose of generating predictions). By virtue of the synthetic data points, one may be able to generate a reasonably accurate output prediction, even for a data point reasonably far away from the data points available in the input data set. The key advantage relates to physics-based nature of the approach. The key disadvantage pertains to the possibility of the approach yielding inaccurate results, if the synthetic data points from CALPHAD are inaccurate, due to limitations (deficiency?) of the CALPHAD databases.

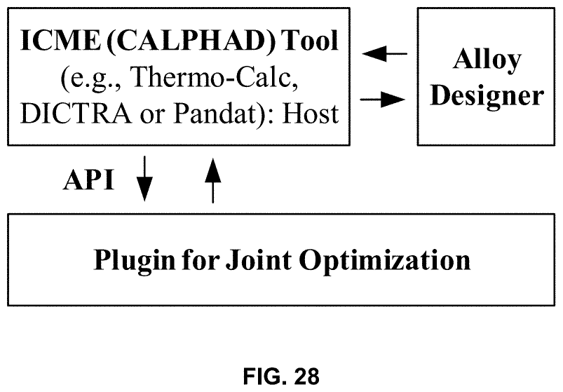

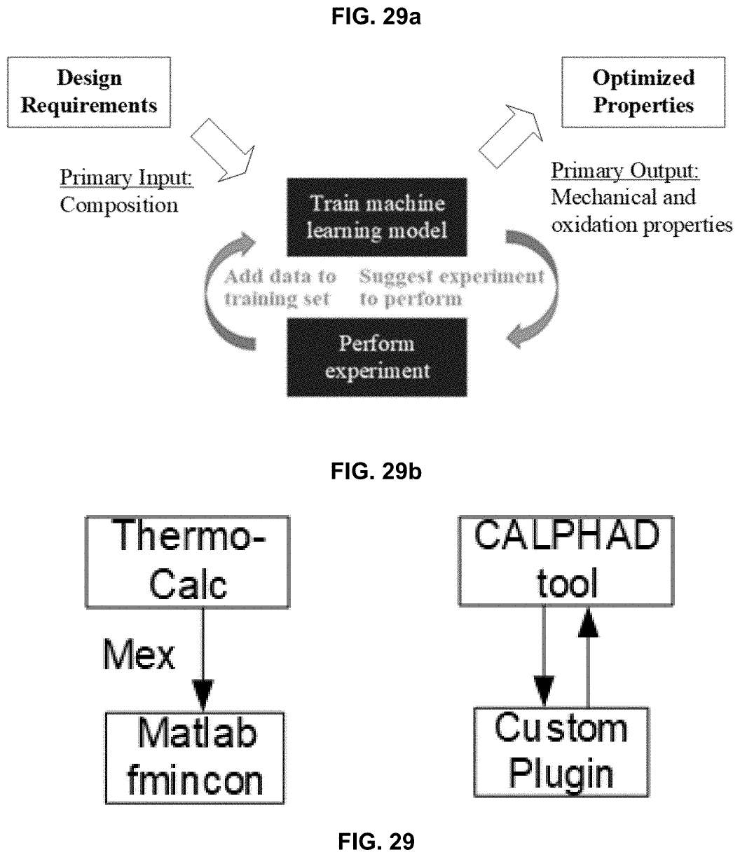

FIG. 28 and FIG. 29 outlines our high-level strategy for joint optimization of the HEA properties. In principle, there are two, primary routes for formulating the joint optimization problem:
1. Through maximization of a joint function or separate functions, accounting for constituent objectives.
2. Through maximization of an objective function only, say, capturing the strength, but where the ductility is accounted for in the constraints.

Specifics for our approach to joint optimization of the HEA properties are listed below:
1. One can start out by organizing and curating a database with data from the literature publicly available.

Ref (CouzinieSenkovMiracle 2018), (BorgFreyMohPollock 2020), (SteingrimssonFanKulkarni 2020) and (SteingrimssonFanKulkarni 2021) contain data sets covering the mechanical properties HEAs, which one can harvest as a starting point. One can also utilize custom (not publicly available) data obtained by virtue of our sequential learning approach, such as shown in FIG. 29a, or through collaboration with industrial or academic partners. Here, one can have full insight into the configuration parameters.
2. Unlike (RettigRitterHelmer 2015), our approach assumes use of high-throughput implementation of CALPHAD.

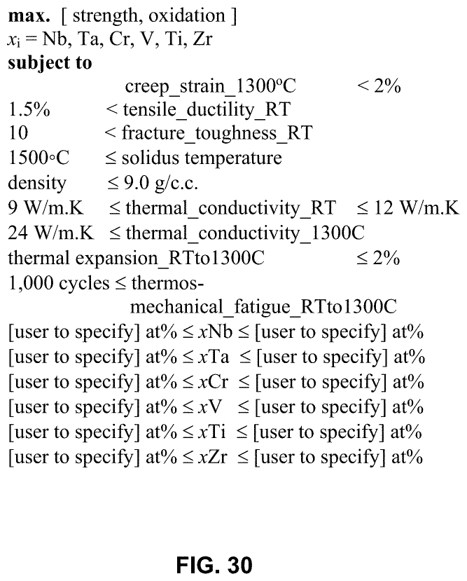

For background information on high-throughput implementation of CALPHAD, refer to (FengZhangGaoPei 2021). In traditional CALPHAD, the designer can only change the ratio of a single element. But in high-throughput CALPHAD, properties, such as liquidus melting point, can be obtained by altering two or more elements at the same time. In high-throughput CALPHAD, models for thermodynamic equilibrium or non-equilibrium can be applied to compute tens of thousands of CALPHAD data points, which can greatly accelerate the design of HEAs with specific properties.
3. One can complete an initial, closed-form formulation of a multi-objective optimization problem for each of the compositions considered, as shown in FIG. 30.

FIG. 30 presents a sample of a closed-form multi-objective optimization problem capturing representative design requirements. The formulation and solution of the optimization problem is specific to the compositions considered and the associated phase properties.
4. One can start with a matlab implementation then move on to a customized implementation mimicking (RettigRitterHelmer 2015).

The approach of (RettigRitterHelmer 2015) is based on a numerical multi-criteria global optimization (a multi-start solver using Sequential Quadratic Programming), which delivers the exact optimum considering all the constraints. The CALPHAD method will be used to provide the thermodynamic equilibrium properties and the creep strength of the alloys will be predicted based on a qualitative numerical model considering the solid solution strengthening of the matrix with the elements involved, the optimum morphology, and the associated phases. The calculated alloy properties, which are required as input for the optimization algorithm, may be computed using very fast Kriging surrogate models.
5. Customization of numerical models, for HEA candidates considered and the associated phases.

Ref (RettigRitterHelmer 2015) presents in Eq. (1) a semi-empirical equation for room-temperature density from Caron et al. (Caron 2000), together with models for lattice parameters of the γ and γ′-phases at room temperature (in Eq. (3) and (4)), thermal expansion of both phases (in Eq. (5) and (6)) and the solid solution strengthening index (in Eq. (7)). Similar semi-empirical relations can be derived for each HEA candidate considered and the associated phases.
6. Pursuit of a sequential learning approach, such as outlined in FIG. 29, and execution of iterative refinements.

A key to success involves incorporation of physics-based insights into the problems. T₀ this effect, the optimization is not carried out in “brute-force” fashion. The HEA candidates in Table 10 were identified optimizing specific parameters, such as the density, solvus temperature, Poison ratio and shear modulus.
7. Verification of the predictive capability of the optimization scheme.

Per FIG. 29, one can verify and refine the predictive capability of the joint ML optimization scheme by sequential learning. One can print materials in relevant geometries from down-selected compositions predicted by the ML algorithm to perform well in elevated temperature creep environments. One then can conduct rigorous high-temperature characterization of the mechanical, creep and oxidation properties of these representative components (coupons). Comparison of predicted yield strength, plasticity and solvus temperature with observed will enable assessment of the predictive capabilities of the machine learning algorithms.

Note that the approach of (RettigRitterHelmer 2015) has been proven for high-temperature modeling (up to 1,100° C.) of super-alloys. The authors, further, state the method should be capable of handling more complex alloys with 12+ elements (as may be the case with HEAs). The solution feasibility of (RettigRitterHelmer 2015), in terms of complexity for nowadays workstations, has also been proven. Our sequential learning approach aligns with authors' statements regarding their approach supporting, but not replacing, conventional alloy design and requiring extensive knowledge of underlying metallurgy.

As an alternative to the approach of (RettigRitterHelmer 2015), one can incorporate ductility using an implementation similar to (GangulyDatta 2007). Here the property functions are modelled by neural networks that rely on large experimental databases. For fall-back, one can also consider a forward-backward prediction scheme, such as presented in (SteingrimssonFanKulkarni 2020) and (SteingrimssonFanKulkarni 2021).

### 5.2 Extension of (RettigRitterHelmer 2015) to HEAs Utilizing DFT in Addition to CALPHAD

Since DFT is physics-based (relies on fundamental quantum mechanical first-principles and atom-level computation), it tends to produce more accurate synthetic data points than CALPHAD, which can be used to improve ductility, yield strength and thermal stability of the candidate RHEAS.

The hybrid approach outlined here is largely identical to the CALPHAD approach from the previous Section 5.1, but with the exception that DFT is utilized in conjunction with CALPHAD to produce a larger number of, and more accurate, synthetic data points than obtained from CALPHAD alone, and to make the most of the data available. The approach extracts relevant quantities from CALPHAD and combines with empirical rules approximating specific mechanical properties of given materials.

### 5.3 Hybrid Extension of (RettigRitterHelmer 2015) and (ConduitJones 2017) Combining the Strengths

The CALPHAD databases for HEAs, needed for the method of (RettigRitterHelmer 2015), may be incomplete to an extent. But the experimental data needed for the method of (ConduitJones 2017) may be incomplete to an extent as well, especially in the case of HEAs. Therefore, it makes sense to combine the methods, such as to make the best use of the CALPHAD, DFT and experimental data available.

### 7.6 Continuation in Part: Manufacturing of Alloys for High-Temperature Applications (Additive Manufacturing Vs. Conventional Casting)

Most turbine blades are made of superalloys and manufactured by investment casting, also referred to as conventional casting (WikipediaTurbineBlade 2021). Directional solidification (WikipediaDirectional Solidification 2021) and single-crystal (WikipediaSingleCrystal 2021) production methods comprised major breakthrough in manufacturing of turbine blades, at the time of their introduction. These methods helped greatly increase the material strength against fatigue and creep, by aligning grain boundaries in one direction, in case of directional solidification, or by eliminating the grain boundaries altogether, in case of the single-crystal technique. Blades presently used in commercial land-based turbines are usually manufactured using conventional casting or directional solidification (Kulkarni 2021). While single-crystal turbine blades do help in terms of reliability (in terms of avoiding defects), they are more expensive to manufacture, and may mostly be needed for air-borne turbine applications (more so than land-based). FIG. 24 presents a representative diagram showing the cooling mechanisms in a turbine blade.

There have been concerns expressed about certification of AM fabricated components and use for safety critical applications. Given the high rotational speed of turbine blades (close to 10,000 mph), presence of defects may result in turbine blades coming apart. Additive manufacturing is considered by some to consist of a weld, on top of weld, on top of weld, etc. Each weld needs to be of high quality, for the overall component to retain high quality. AM can result in multiple types of defects, including key holes, lack of fusion, open pores (porosity), hot-short cracking, segregation (unmelted particles), residual stresses and fatigue cracks. Based on the build direction, it is also possible that defect characteristics exhibit dependence on the component geometry. With such concerns indeed having some merit, it is highly likely that the AM components certified first will consist of stationary components. More extensive data collection and design analysis may be needed for rotating components. Even so, Siemens has successfully validated multiple AM printed turbine blades with a conventional blade design at full engine conditions (T&DworldSiemens 2021). Many turbine vendors, such as Siemens, have developed a deployment plan for adoption of AM components.

SLM beats casting in time. But for high volume, casting offers superior benefits. Furthermore, AM (including SLM) can support hierarchical microstructures, but not casting. Hierarchical microstructures can give rise to components with superior properties (ZhuNguyenNgAn 2018). Turbine vendors, like Siemens, we have done casting for a number of years, so there is a procedure in place for certifying parts for quality. SLM, on the other hand, is still undergoing evolution.

The evolution applies primarily to the AM process parameters, but can also apply to the compositions. In case of Haynes 282 superalloy fabricated through SLM, no tweaking of the composition may be needed. Appropriate tweaking of the post-processing (heat treatment) may suffice. But in case of wire & arc AM of Haynes 282 superalloy, some tweaking of the composition may be needed.

In case of refractory HEAs, the choice of using conventional casting or additive manufacturing for the fabrication, would depend on the HEA composition involved. If a casting process is already available for that composition, then a casting process may be a preferred starting point.

As for manufacturing of a prototype turbine blade from HEAs, vendors like Siemens are still in the early stages of exploring fabrication through vacuum melting. Once they have defined such a fabrication process, they will likely investigate the potential of fabrication through casting or SLM and compare properties. Preliminary results suggest that HEA materials manufactured using AM tend to exhibit comparable or better properties than materials fabricated using conventional drop cast technology.

In terms of the primary challenges involved in extending conventional casting to AM (say, SLM), the primary challenges are expected to depend on the composition involved. It may be hard to change a casting process, without defining the chemistry of the HEAs.

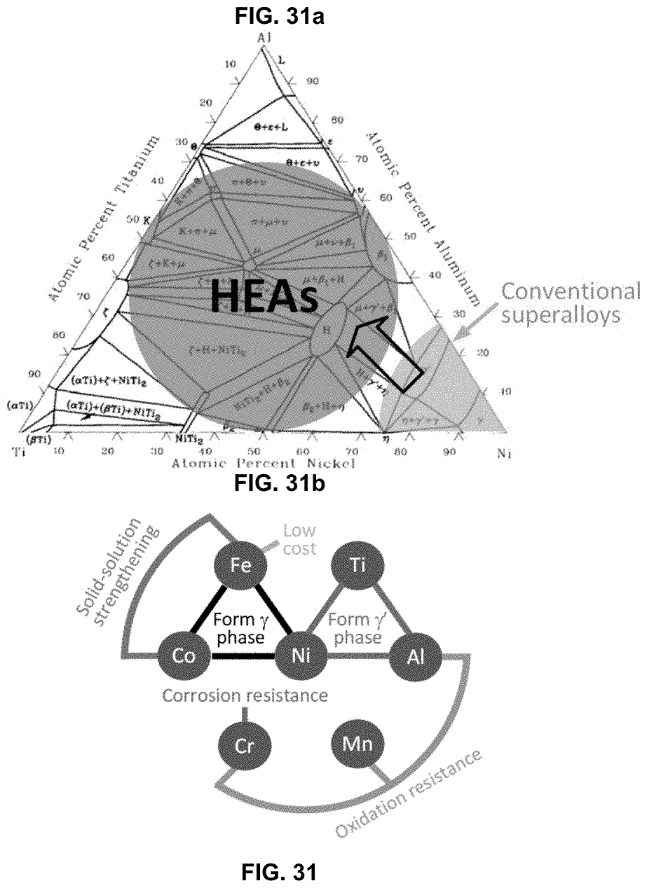

For a review on the fabrication of metallic, single-crystal turbine blades, with a commentary on repair via additive manufacturing, refer to (AngelBasak 2020). For additional information on high-temperature alloys for gas turbines, refer to (Brunetaud 1982). For additional information on the advantages of AM, such as efficiency, the ability to produce near-net-shape components (components with high buy-to-fly ratio), and to overcome geometric limitations (ability to produce highly complex structures), refer to (SteingrimssonFanKulkarni 2021). Table 4.2 of (SteingrimssonFanKulkarni 2021) contains comparison between powder feed AM and powder bed AM processes. Table 4.3 of (SteingrimssonFanKulkarni 2021) similarly provides comparison between SLM, EBM and WAAM metallic AM processes. FIG. 31 illustrates rational for determination of elements for a superalloy composition, and visualizes how HEAs can be viewed as an extension of conventional superalloys.

### 7.7 Continuation in Part: Defects in Additive Manufacturing (with Emphasis on Hot Cracking)

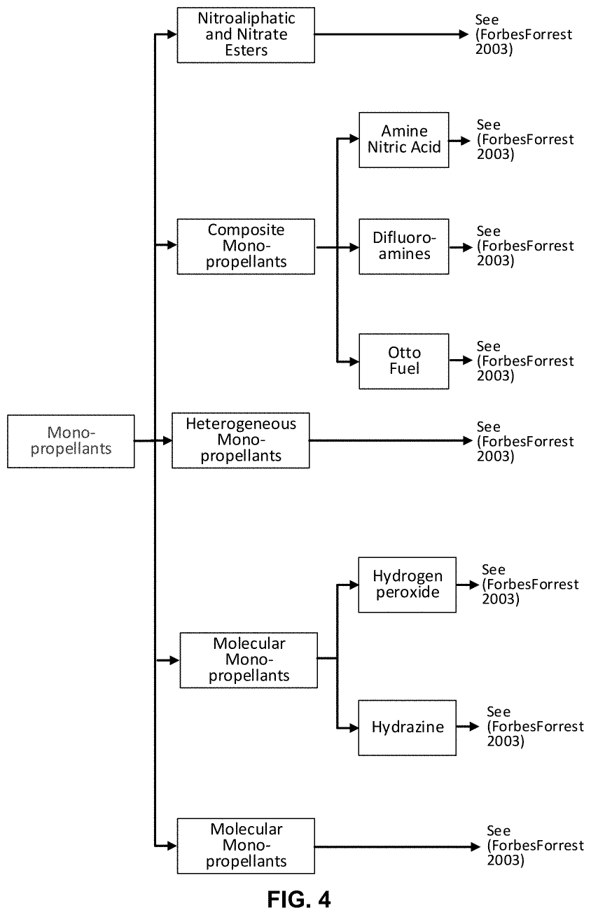

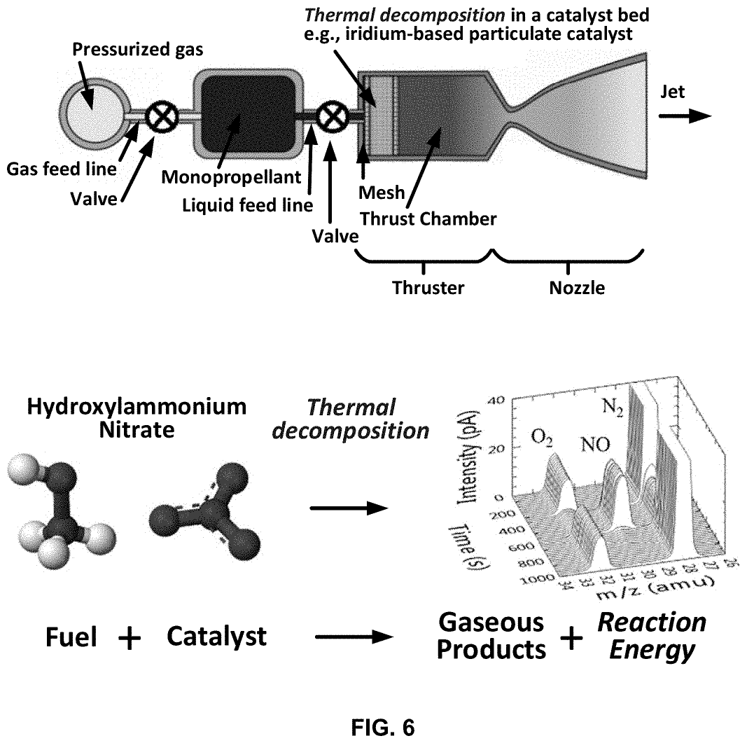

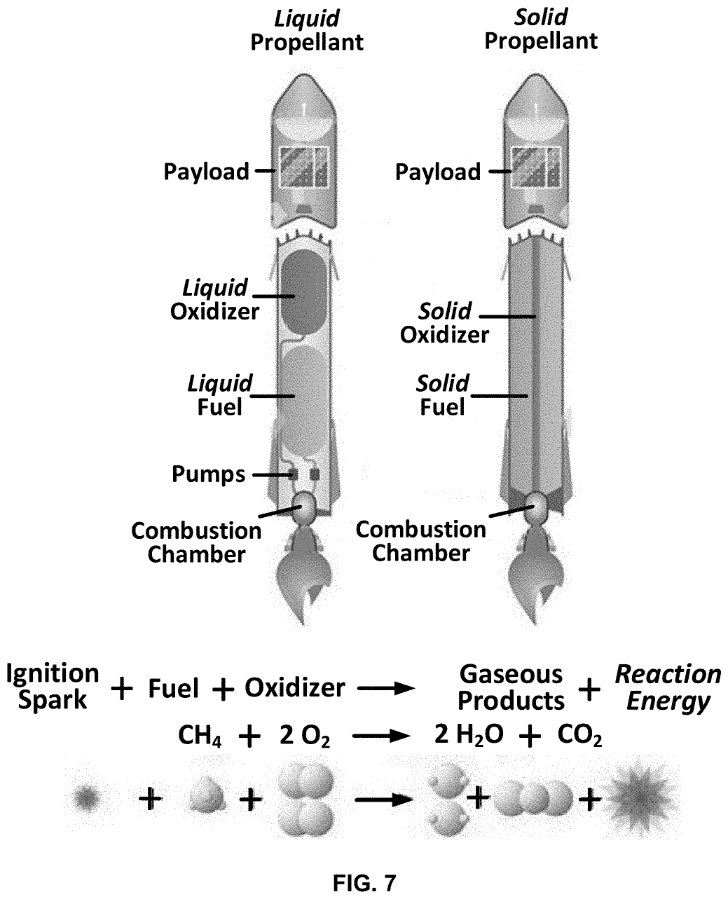

Components produced by AM from superalloys have been prone to defects. There is desire to develop alloy compositions that enable AM processes to produce properties that are not currently achievable such as materials with a preferred crystallographic orientation, dispersion-forming alloys that can either form the dispersion during AM or after AM through heat treatment. Alloy compositions or process parameters should reduce defects in components, thus promoting them to be more resistant to fatigue, with potential increases in strength. Expanding on FIG. 4, FIG. 6 and FIG. 7 from (SteingrimssonFanKulkarni 2020), the following types of defects are primarily observed in powder bed AM:

1. Key hole.
2. Lack of fusion.
3. Open pores (voids or porosity).

### 4. Inclusions.

5. Segregation (or unmelted powder).
6. Fatigue cracks.
7. Hot-short cracking.

The keyhole defects consist of spherical type of defects that can happen, when one supplies excessive energy to the sample (more energy than required to melt the powder).

If one supplies too little energy to the sample fabricated, lack-of-fusion defects can result. The lack of fusion defects are characterized by sharp corners (some high aspect ratios). Lack of fusion defects depend mainly on the size and shape of the melt pool (the melt pool geometry), not on the cooling rate.

Open pores or porosity can result from trapped gas, when the energy source used with the powder bed AM provides too much energy, and when the cooling takes place too rapidly. Gas porosity from powders and thermal shrinkage pores are common in as-built samples. Defects, including pores, tend to adversely affect the mechanical properties of additively manufactured components or samples. According to (WilsonHeidNovakBeese 2019), intentional pores began to impact ultimate tensile strength of austenitic stainless steel 316L manufactured with laser powder bed fusion, when the pore diameter was 2400 μm, or 16% of the cross-sectional sample area. Elongation to failure was considerably affected, when the pore diameter was 1800 μm or 9% of the cross-sectional sample area. This illustrates that 316L stainless steel manufactured by AM is defect-tolerant under uniaxial tension loading.

Similar to voids and porosity, inclusions can refer to trapped gas (e.g., oxygen inclusions). But an inclusion can also refer to a trapped foreign, non-gaseous particle (e.g., oxide inclusions or carbide inclusion). Inclusions usually refer to trapped solid, non-gaseous particles or impurities, like silicon carbide.

Segregation refers to chemical segregation, when different elements segregate as a result of unmelted particles. When fabricating refractory HEAs, some of the powders with the highest melting point may not have fully melted.

Fatigue cracks can result from cyclic loading as a pre-cursor to failure. Cracking in AM parts can also consist of hot (or hot-short) cracking, solid-state cracking, strain-age cracking, solidification cracking, liquation cracking or ductility dip cracking. Hot cracking and solid-state cracking results from process cracks due to thermal gradients and metallurgical considerations. Solidification and liquation cracking can be considered subcategories within hot-short cracking.

Hot-short cracking usually results from segregation during solidification. If the inter-dendric region has low melting temperature, the whole part cools. But it also starts to contract, because of the thermal stresses. Therefore, you can have regions, which are not fully solidified yet, because of very large freezing range, but where the thermal stresses will pull apart and generate a hot-short crack. Hot-short cracking commonly results from compositional segregation and resultant local depression of effective liquidus.

The combination of large temperature gradients and rapid cooling results in high levels of stress that can lead to hot cracking in many of the high temperature stable alloys. Furthermore, the large thermal gradient in the molten pool prevents nucleation ahead of the solidification front which leads to residual stress, columnar growth, and cracking across the previous deposited layers.

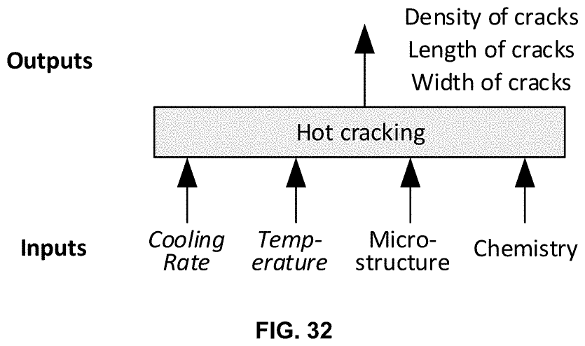

In terms of influencing factors for solidification cracking, shown in FIG. 32, it is generally the solidification temperature range (temperature between liquidus and solidus), the amount and composition of inter-dendritic liquid in the last stages of cooling. The cooling rate can then further exacerbate these factors.

In terms of strategies for mitigating cracking, (ChauvetKontisJagle 2018) showed that hot solidification cracking (hot tearing) susceptibility is related to angle of grain boundaries, with high-angle grain boundaries (those with misorientation >15°) more affected than low-angle grain boundaries (those with misorientation <15°), possibly because of higher potential for segregation and stress accumulation. The low-angle grain boundaries exhibited higher fraction of grain boundaries and thus more distributed residual stress. Chauvet et al. showed that the presence of micron sized borides with stoichiometries corresponding to MB, M2B and M5B3 suggests a significant local enrichment in B in the vicinity of the grain boundaries (ChauvetKontisJagle 2018). Local compositional enrichment by minor elements, in particular B, are thought to play a significant role to maintain a liquid film at lower temperature in comparison with the theoretically assumed equilibrium solidus temperature. An extensive study by Harrison et al on Hastelloy X, which otherwise was weldable, showed that was not possible to attain crack-free material by only changing process parameters (HarrisonToddMumtaz 2015). However, small increases in solid-solution strengthening elements and control of impurities resulted in significant crack reduction (HarrisonToddMumtaz 2015) Carter et al. demonstrated that reduction in scan spacing resulted in lowering of voids as well as tight control of laser power density (CarterAttallahReed 2012). Post-process hot isostatic pressing was needed to mitigate cracks as much as possible (CarterAttallahReed 2012).

In terms of tools for simulation and design of AM components, Siemens has developed advanced tools for simulation and design of AM components to address the defects within complex geometries early in the process to achieve defect free components (FuHaberlandKlapdorRule 2017). Siemens has printed defect free parts that are operating in engines and test rigs (T&DworldSiemens 2021). For difficult to weld alloys, sluggish melt pool, along with ductility behavior of the alloy, may provide insight into parameter space that enable processing.

### 7.8 Continuation in Part: Scheil Model for Solidification of an Alloy

The Scheil equation describes the concentration of solute in a solid, Cₛ, and the composition of liquid, C_{L}, during solidification, as a function of the fraction solidified, fₛ. Four primary assumptions of the Scheil model enable determination of the solid phases present in a cast part (WikipediaScheilEquation 2021):
1. No diffusion is assumed to occur in the solid phases once they have formed (Dₛ=0).
2. Infinitely fast diffusion is assumed to occur in the liquid at all temperatures, by virtue of a high diffusion coefficient, thermal convection, Marangoni convection, etc. (D_{L}=∞).
3. Equilibrium is assumed to exist at the solid-liquid interface; in other words, compositions from a phase diagram are assumed to remain valid.
4. Solidus and liquidus are assumed to consist of straight segments.
   According to (WikipediaScheilEquation 2021), the fourth condition may be relaxed when numerical techniques are used, such as those employed by CALPHAD software packages.

The Scheil model states that the concentration of solute in a solid, Cₛ, depends on the initial concentration of the liquid, C₀, as

Cₛ=k C₀(1−fₛ)^{k-1} (72)

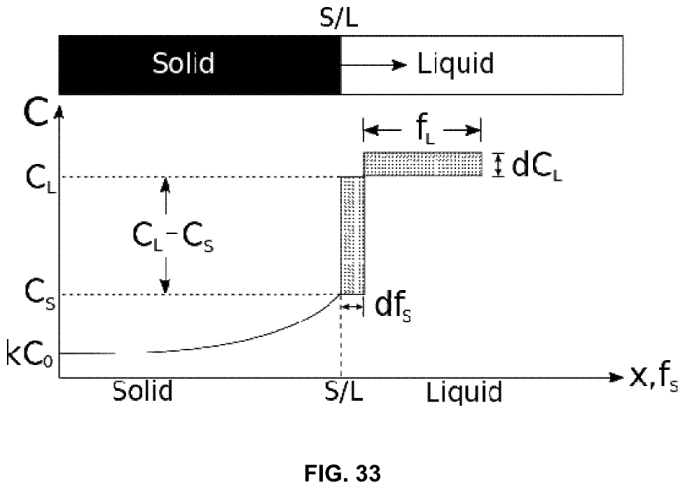

Here, fₛ represents a fractional distance along the bar, and k is a partition coefficient. FIG. 33 describes the process of solute redistribution during solidification, according to the Scheil model. For full derivation of the Scheil model, and more detailed graphical illustrations of the solidification process, refer to (WikipediaScheilEquation 2021), (UCambridgeScheilEq 2021).

### 8. Verification, Validation and Reporting

### 8.1 Verifying Predictive Capability of ML Algorithms

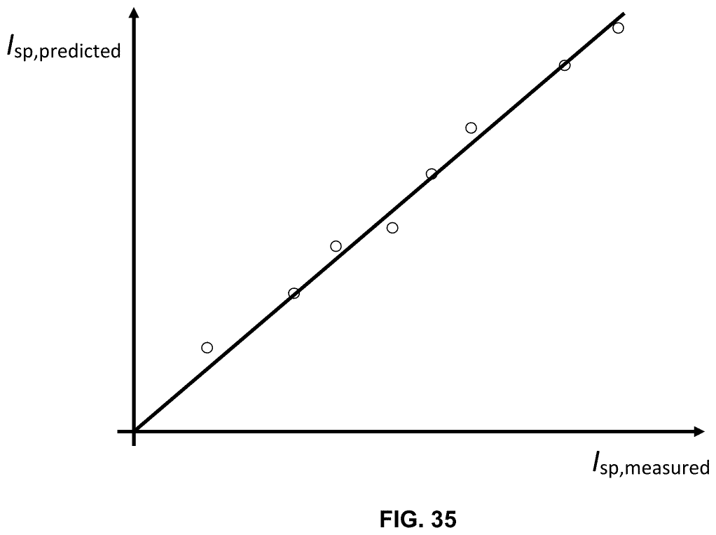

Verification of the predictive capability of the ML algorithms is primarily based on comparison with experimental results. FIG. 35 shows how the predicted values for the specific impulse can be compared to the observed (measured) values. Comparison of predicted properties of the energetic materials to the observed properties enables assessment of the predictive capabilities of the machine learning algorithms. One can measure the coefficient of determination (R²) relative to the red diagonal.

In case of the alloys, validation and verification may entail prediction of RHEA compositions and properties that allow for comparison with known quantities from the literature.

### 8.2 Analysis of Outliers Verification Addressing Implications of Incomplete Data Sets

FIG. 36, Table 14 and Table 15 of (SteingrimssonFanKulkarni 2020) as well as Section 4.4.7.6 of (SteingrimssonFanKulkarni 2021) illustrate how outliers, resulting from a limited data set can be analyzed.

### 8.3 Approach to Rapid Screening

For rapid screening (high throughput experiments), one may emphasize properties that do not depend heavily on the molecular structure.

### 8.4 Uncertainty Quantification

We assume the uncertainty quantification is consistent with similar activities within NIST. NIST has some interesting projects for AUML data extraction and uncertainty prediction, such as AI self-quality assurance using learning curves in feedback loops (NistAiUncertainty 2019).

### 8.5 Reporting

The reporting mechanism is integrated into the user interface outlined above. In one embodiment of the invention, reporting of results predicted is based on the traditional Model-View-Controller paradigm (Steingrimsson 2017).

### 9. System Integration and Interfacing with External Design Tools

The apparatus captures a software application for optimization of the properties of energetic materials. There are two primary embodiments of the invention:

1. Method and apparatus comprising of a host software application.
2. Method and apparatus comprising of an embedded implementation.

In case of the host software application, specific software packages can be launched, for the purpose of obtaining specific data, in a fashion analogous to (SteingrimssonKulkarni 2020). One can launch calculations involving thermo-physical, thermo-chemical or molecular dynamics simulations from the host application in a similar fashion. Such simulation tools (other tools) can even be incorporated into the stand-alone host software application.

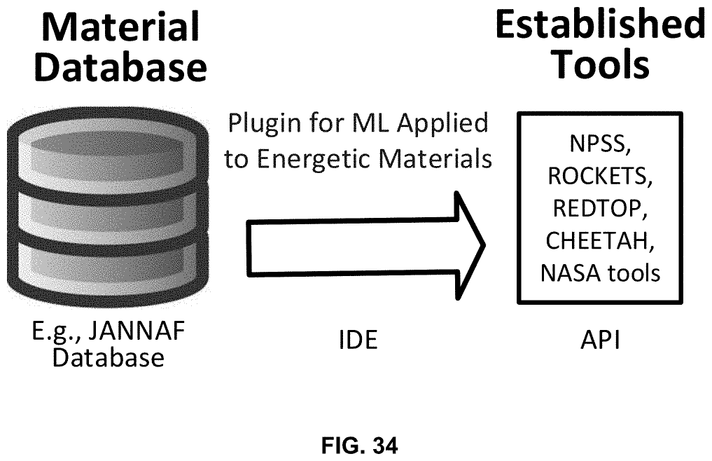

In case of the embedded implementation, the invention involves a plugin or add-on, or even a web service, per FIG. 34, for established tools used for the design of energetic materials, such as Cheetah, NASA CEA software, NPSS or ROCKETS REDTOP. The plugin or add-on can accommodate toolboxes (or libraries) for machine learning, artificial intelligence or data analytics, including the TensorFlow package (TensorFlow 2020) or scikit-learn (SciKit-Learn 2020) The physics-based models may be incorporated as add-ons to open-source, off-the-shelf toolboxes (libraries) for machine learning, artificial intelligence or data analytics, to provide physical insight as unexplored sections of the composition space are navigated.

In terms of software library interfaces, LAMMPS can be built as a library (static or shared), so that it can be called by another code, used in a coupled manner with other codes, or driven through a Python script. Even the LAMMPS standalone executable is basically a thin wrapper on top of the LAMMPS library, creating a LAMMPS instance, processing input and then existing, LAMMPS provides four types of APIs (LammpsLibraryInterfaces 2021):

### 1. LAMMPS C Library API.

### 2. LAM MPS Python APIs.

### 3. LAM MPS Fortran API.

### 4. LAMMPS C++ API.

Most of the APIs described in (LammpsLibraryInterfaces 2021) are based on C language wrapper functions in the files src/library.h and src/library.cpp. But it is also possible to employ C++ directly. The basic procedure remains the same: One can create one or more instances of LAMMPS, pass commands as strings or from files to that LAMMPS instance to execute calculations, and/or call functions that read, manipulate, and update data from the active class instances inside LAMMPS to conduct analysis or perform operations that are not possible with existing input script commands.

The C library API comprises the most commonly used approach for managing LAMMPS instances from a compiled code and it is the basis for the Python and Fortran modules. Just about all functions of the C language API require an argument containing a “handle” in the form of a void*type variable, which points to the location of a LAMMPS class instance (LammpsLibraryInterfaces 2021).

A LAMMPS library.h header file by default does not include an mpi.h header file and thus conceals the lammps_open( ) function which requires the declaration of the MPI_comm data type. This can only cause a problem when the communicator that would be passed differs from MPI_COMM_WORLD. Otherwise, calling lammps_open_no_mpi( ) is expected to work just as well. To make lammps_open( ) available, one needs to compile the code with the flag-DLAMMPS_LLB_MPI enabled or add the line #define LAMMPS_LIB_MPI before #include “library.h” (LammpsLibraryInterfaces 2021).

The LAMMPS Python module provides the ability to call the LAMMPS C library API from Python by dynamically loading functions in the LAMMPS shared library through the Python ctypes module. Due to the dynamic loading, it is required that LAMMPS is compiled in “shared” mode. The Python interface is object oriented, but otherwise looks similar to the C library API (LammpsLibraryInterfaces 2021).

The LAMMPS Fortran module is a wrapper around the LAMMPS C library API. This is accomplished by utilizing the ISO_C_BINDING feature in Fortran 2003. The interface is object oriented but otherwise looks similar to the C library API and the basic Python module (LammpsLibraryInterfaces 2021).

It is also possible to invoke the LAMMPS C++ API directly from the source code of the plugin, add-on or host application. The LAMMPS C++ API lacks some of the convenience of the C library API, but allows for more direct access to simulation data and thus enhanced low-level manipulations (LammpsLibraryInterfaces 2021).

Similar to its predecessor, the TIGER thermochemical code, the Cheetah provides a command line interface originally written in Fortran. Cheetah supports a startup file that can load initialization commands automatically. It also supports a reactant database containing frequently used explosives and binders (multiple product libraries). The output of Cheetah can be exported to a spreadsheet file and imported into Excel (LlnlCheetah 2021).

The NASA CEA program was written in ANSI standard Fortran 77. The NASA CEA program consists of the following five files:

1. The source program (cea.for).
2. Thermodynamic data (thermo.inp).
3. Thermal transport properties (trans.inp).
4. Sample problems (sea.inp).
5. User documentation (readme.txt).

After the cea.for file has been compiled, unprocessed thermodynamic and transport property data is expected to be processed once. The processed data is stored in the files thermo.lib and trans.lib in binary form, where they remain available for future use (CeaUserManual 2021).

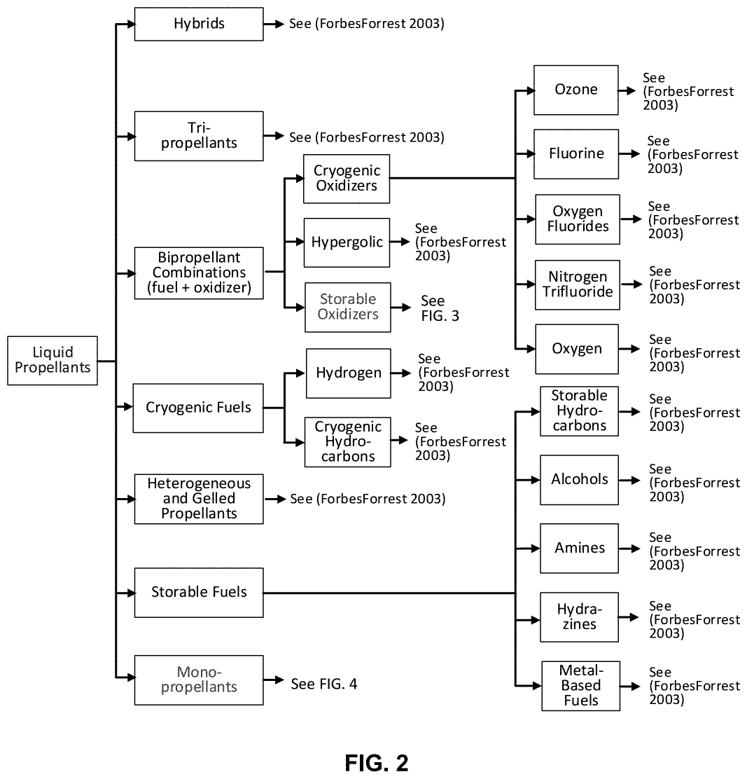

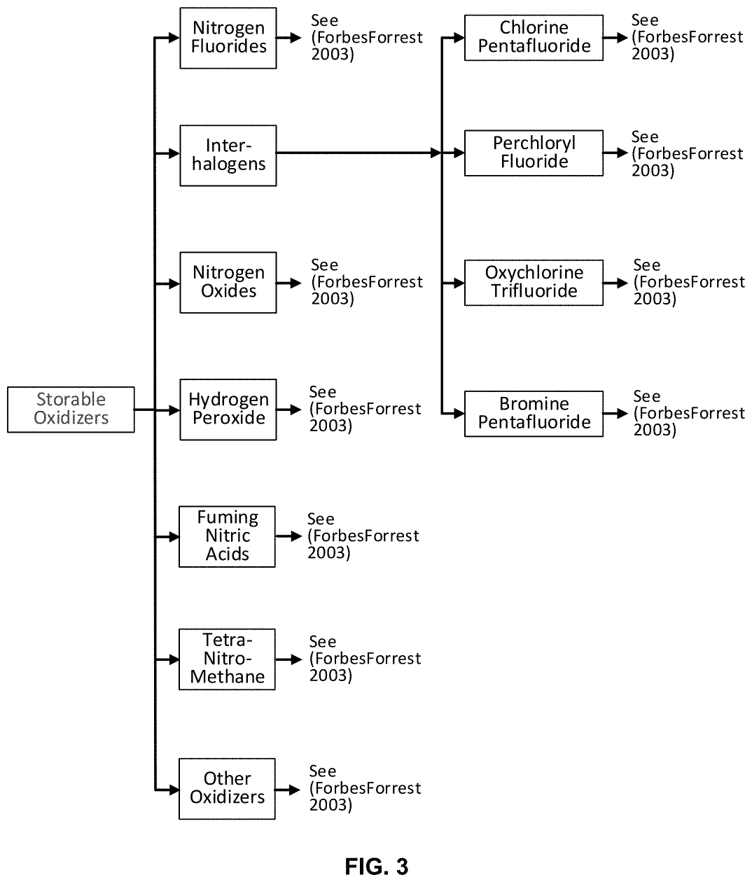

FIGS. 3.1 and 3.2 of (HallRascheSimons 2006) present flow charts for the NPSS a multi-disciplinary Numerical Propulsion System Simulation environment. FIG. 3.1 depicts what is referred to as hot-to-cold coordinate conversion process (Stage 1 deflection analysis of airfoils). Stage 2 of the overall analysis, illustrated in FIG. 2, provides deflection analysis of aerodynamic prediction for off-design operation of airfoils. NPSS is not a single software application, but a simulation environment for computer aided design, one that can invoke tools for solid modeling, structural analysis and computational fluid dynamics. Appendix B of (HallRascheSimons 2006) presents a NPSS Stage 1 ANSYS macro. Appendix C of (HallRascheSimons 2006) similarly contains a NPSS Stage 2 ANSYS macro.

### 10. How to Make the Invention

The objective of the invention is to accelerate the design of energetic materials, primarily propellants, exhibiting high energy density, defined as energy produced per unit mass of propellant. In practical terms, this usually translates into optimization of a specific impulse, a density specific impulse, detonation pressure or detonation velocity, as noted above. Data-driven approaches provide new opportunities to accelerate the discovery of such energetic materials.

The invention is made by constructing the prediction engine in FIG. 13.

### 10.1 User Interface

In case of a prediction engine employing statistical modeling, such as for predicting the energetic properties in particular the energy density—of propellants, explosives or pyrotechnics, the preferred embodiment of the invention assumes the design of an efficient user interface, one capable of effectively guiding the user through (effectively helping the user specify) the multiple parameters impacting the energetic properties.

### 10.2 Database System

The database system is constructed in accordance with the preferred embodiment outlined above.

For the purpose of identifying and extracting physio-chemical data sets suitable and archival into database, one can populate a test SQL data base with material records resembling the ones from the PubChem database (PubChemDatabaseAPI 2021), the NIST database (NISTdatabase 2021), the NIST ICSD database (ICSDdatabaseAPI 2021) or the JANNAF database (JANNAF 2021), (JannafThermoChem 2021), using matlab.dat files or .xlsx or .csv files from Excel available. These databases may provide a good starting point for a classification scheme with the following structure:

Universe→Family→Class→Member→Attributes→Material Records.

The structure of the data in PubChem database (PubChemDatabaseAPI 2021), the NIST database (NISTdatabase 2021), the NIST ICSD database (ICSDdatabaseAPI 2021) or the JANNAF database (JANNAF 2021), (JannafThermoChem 2021) seems to map well to relational databases.

In one embodiment of the invention, the feature extraction may resemble the Citrination platform for materials science data (LingAntonoBajaj 2018), (O'MaraMeredigMichel 2016). The Citrination platform automatically parses chemical formulas and alloy compositions, calculating over ninety different features based on the elemental properties (e.g., ionization energy, melting temperature and the number of valence electronics).

The data imported can comply with Structured Query Language or JavaScript Object Notation. Alternatively, the data may be ingested in the form of delimited text, multi-line data, or in the XML, YAML, HTML, KML, JSON, GeoJSON, CSV, Excel, JSON, SQL, DB, DBs or FlatFile formats.

### 10.3 Prediction Logic

The prediction logic receives primitives from the prediction engine and passes to the reporting and validation module. The primitives include the quantity predicted, such as the feature list.

### 10.4 Prediction Engine

The prediction engine is constructed in accordance with the preferred embodiment outlined above. For accurate prediction of an output quantity, it is essential to properly account for the input sources that contribute to variations in the output. Properly accounting for the input sources is a primary determinant of the prediction accuracy. Further, it is of paramount importance to have access to complete and properly curated data sets.

In one embodiment of the invention, the procedure for constructing the prediction engine may consist of the following steps:
1. One can start out by populating a sample SQL database with pertinent materials data, such as from the PubChem database (PubChemDatabaseAPI 2021), the NIST database (NISTdatabase 2021), the NIST ICSD database (ICSDdatabaseAPI 2021) or the JANNAF database (JANNAF 2021), (JannafThermoChem 2021).
2. One can normalize the data, if necessary, and then derive features in accordance with Eq. (23) Eq. (25) or Eq. (30) Eq. (32).
3. One can train the prediction model on pertinent materials data, such as from PubChem database (PubChemDatabaseAPI 2021), the NIST database (NISTdatabase 2021), the NIST ICSD database (ICSDdatabaseAPI 2021) or the JANNAF database (JANNAF 2021), (JannafThermoChem 2021), and expand from there.
4. For the prediction, i.e., for deriving a solution to the optimization problems listed in Eq. (26), Eq. (27), Eq. (29), Eq. (33) or Eq. (36), or for optimization of the other output quantities listed in Section 7.1, one can employ the algorithms outlined in Sections 7.2, 7.3 or 7.4 above on in (SteingrimssonFanKulkarni 2020), (SteingrimssonFanKulkarni 2021).

The prediction model is selected in accordance with the data available and the application at hand. For a data set with Nₜᵣₐᵢₙ samples allocated to training of the prediction model, one may only afford to select a model with the number of unknown parameters, Nₚₐᵣₐₘ, satisfying

Nₚₐᵣₐₘ<Nₜᵣₐᵢₙ. (72)

Keep in mind that in order to reliably estimate a line (a model with two unknown parameters), one needs at least two distinct data points. Similarly, in order to reliably estimate a plane (a model with three unknown parameters), one needs at least three distinct data points. In the case of a neural network, net, trained in MAT LAB®, the number of unknown parameters can be estimated as

Nₚₐᵣₐₘ=length(getwb(net)). (73)

Similar to the note above, the suitability of an optimization technique for the data available and a problem at hand depends in part on the number of unknown parameters in the technique relative to the number of data points available to estimate the model implemented by the technique. For further specifics, refer to (SteingrimssonFanKulkarni 2020), (SteingrimssonFanYang 2021) and (SteingrimssonFanKulkarni 2021).

The quality of prediction can be assessed by computing the coefficient of determination, R², per FIG. 35, from a graph showing I_{sp,predicted} against I_{sp,observed}.

Assessment of the extent of the input set needed for estimating the quantities predicted with a given level of accuracy comes down to estimation of confidence intervals for the parameters of the probability density function (PDF) that given ML algorithms are seeking to approximate. For specifics, refer to Section 4.2.2.5 of (SteingrimssonFanKulkarni 2021).

### 10.5 Verification, Validation and Reporting

This invention seeks to facilitate use of the database to enable prediction or down-selection of molecules for determination of potential candidates of energetic materials for laboratory synthesis and further considerations.

Determination of existence or non-existence of a composition (solution) fulfilling the design requirements comes down to the prediction accuracy expected and how the requirements are presented. The design requirements should preferably be presented with an associated uncertainty interval.

One can report verification and validation results in the form of scatter plots and error histograms similar to (Ling AntonoBajaj 2018) or (AgrawalDeshpande 2014).

### 10.6 System Integration and Interfacing with External Design Tools

The methods and apparatus, described in this invention, can interface with the external design tools, including LAMMPS, Cheetah, TIGER, the NASA CEA software, NPSS or ROCKETS REDTOP in fashion analogous to Ref. (SteingrimssonKulkarni 2020).

### 11. How to Use the Invention

### 1. For Novel Materials Discovery For Down-Selection of Candidates of Energetic Materials or Alloy Compositions

This invention facilitates technology innovations in the areas of novel materials discovery and composition down-selection utilizing ICME. Here, the most critical, first step entails an ICME approach for optimizing (balancing) the desired properties. Machine learning is able to steer the decision process for a propellant to the best alternative.

### 2. For Accelerating the Development of Energetic Materials (Propellants, Explosives or Pyrotechnics) or Alloys

Machine learning is also capable of accelerating the development of new energetic materials, formulations and molecules, with desired properties, such as with high energy density.

The machine learning prediction framework is presented here as a dual-use technology, intended both for military and civilian use. New energetic materials provide the opportunity to increase the operational capability of space-borne assets. Direct dual use applications of the energetic materials may include a wide range of commercial applications, e.g., within the terrestrial aerospace, the satellite, marine, automotive, nuclear, or the oil and gas industries. Dual-use applications on the alloy side may include aircrafts, land vehicles, ships, submarines or materials processing entities. Tools for accelerating the new material discovery, and optimizing manufacturing processes, may benefit defense warfare centers and production facilities.

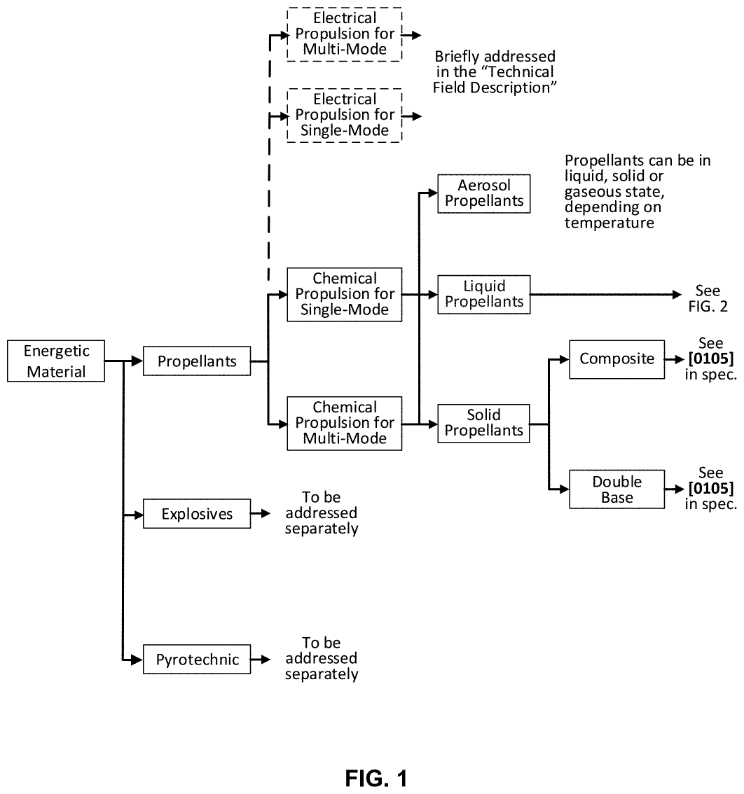

The machine learning is, further, presented as a technology that applies to all of the propellant types listed in FIG. 1 FIG. 4, including monopropellants, bipropellants or hydrogen fuel. The theory of detonation, introduced by Kamlet and Jacobs (KamletJacobs 1968), Chapman and Jourguet (ChapmanJouguet 2021), helps explain how similar technology can be also be applied to explosives and pyrotechnics.

### 3. As a Leading Paradigm in the Design or Application of Energetic Materials

In the past, modeling and simulation efforts have mainly played a supportive role, rather than a leading role in the discovery or design of new energetic materials. Traditionally, the development of energetic materials has primarily been based on test-driven approaches. Continued application of such methodology (test-driven approaches) may not be cost-effective, sustainable, or sufficiently responsive to warfighters' needs. According to (HuangLiTanWen 2021), high hazard levels associated with the traditional methodology, combined with high cost of experimental verification, and long-term life cycle of their characterization, manufacturing, testing, and inspection, could help explain the relatively slow development of HEDMs. The prediction engine presented in this invention may change the methodology employed for developing energetic materials.

### 4. As a Plugin to ICME Tools

In one embodiment of the invention, the prediction engine is employed as a plugin to ICME tools, such as Cheetah, the NASA CEA software, LAM MPS, NPSS or REDTOP, in case of energetic materials, or to Thermo-Calc, DICTRA or the Pandat software, in case of alloy design. In case of an embedded implementation of the invention, host applications used by alloy or turbine designers may also include CFD tools, or tools for crack growth analysis, such as NASGRO, FE-SAFE, nCode DesignLife or AFGROW.

A plugin for joint optimization into established tools used for designing new energetic materials has the potential to greatly accelerate the discovery of new energetic molecules.

### 5. For Reducing the Time Necessary for Development, Testing and Validation of New Alloy Systems and Components, Especially for High-Temperature Applications

In general, integrated computational and experimental approaches are needed that can reduce the time necessary for development, testing, and validation of new alloy systems and components. NASA, for example, has expressed interest in integrated computational and experimental approaches that can decrease the time necessary for development, testing, and validation of new alloy systems and components (NasaVision2040 2021). The integrated modeling tool presented, for development of materials and structures, contributes to NASA's ability to achieve its long-term aeronautics goals, including the development of advanced propulsion systems.

NASA recently sponsored a study to define potential 25-year goal for integrated, multiscale modeling of materials and systems to accelerate the pace and reduce the expense of innovation in future aeronautical systems. The results of this study were published in a NASA report, “Vision 2040: A Roadmap for Integrated, Multiscale Modeling and Simulation of Materials and Systems” (NasaVision2040 2021).

NASA has identified as critical the increased use of modeling tools, such as machine learning and multiscale of modeling tools, for improving R&D effectiveness and for enabling more rapid and revolutionary materials design. This invention combines the strengths of machine learning and multiscale (physics-based) modeling, for purpose of making up for limitations of each individual approach. The computational materials and multiscale modeling tool outlined in the invention falls under the category of Transformational Tools and Technology and Convergent Aeronautics Solutions projects at NASA, which include methods to predict properties, and/or durability of propulsion materials based upon chemistry and processing for conventional as well as functionally graded, nanostructured, multifunctional, and adaptive materials.

### 6. For Accelerating the Development of Advanced Propulsion Systems

In principle, similar plugins for joint optimization of material properties can be used not only for designing energetic materials for multi-mode space propulsion, but also for designing alloys for rocket propulsion, and for designing of alloys for high-temperature gas turbine applications.

This invention contributes to NASA's ability to achieve its long-term aeronautics goals, including the development of advanced propulsion systems. T₀ this effect, the invention addresses the design and development of unique alloy systems, for vehicle propulsion system structures and components. For future aircraft with hybrid-electric or all-electric propulsion systems, advanced materials technology is needed for power components including electric machines and power cables.

### 7. For Enabling New Propulsion Systems with High Levels of Thermal, Transmission and Propulsion Efficiency

This invention addresses design and development of novel alloys, such as for subsonic transport vehicles, with high levels of thermal, transmission, and propulsive efficiency.

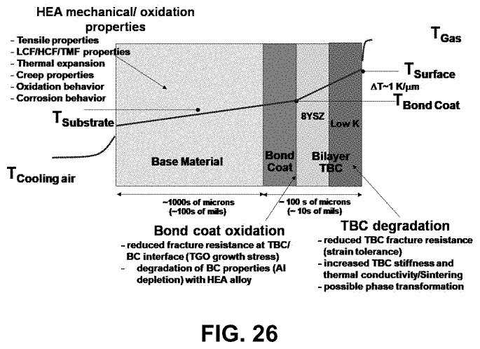

This invention, further, facilitates technology innovations in the areas of novel alloy discovery, composition down-selection utilizing ICME and AM coupled with cooling scheme optimization to increase the material temperature limits, and also to achieve potential blade cooling efficiency improvements. The invention addresses joint optimization of the mechanical properties of HEAs for applications involving turbine blades capable of operating at higher temperature and with greater efficiency, resulting in improved fuel efficiency and reduced emissions. The integrated modeling tool presented contributes to the disruptive and transformative material solution that is needed to replace the Ni-based superalloys with HEAs offering higher materials temperature capability together with enhanced creep, fatigue and oxidation resistance. Design studies show, per FIG. 26, that higher-temperature-capable structural alloys like HEAs allow for 50% reduction in first stage blade cooling. Per FIG. 23 and FIG. 24, an ultrahigh capable RHEA can provide a minimum target improvement in cooling efficiency of 50%, leading to a 1-1.5% increase in efficiency and a 2-4% increase in power output.

The invention has the potential to result in reduction in fuel consumption for Siemens SGT-A05 aero-derivative gas turbine fleet by 71,505 ton/yr., as noted above. A 7% improvement in efficiency in the natural gas turbines used for electricity generation (for civilian aircraft) represents saving up to 15-16 quads (3-4 quads) of energy (NewellHagerty 2020), (FederalAviation 2020), (BureauTransportation 2020).

### 8. For Design of Alloys for Use in Energy Conversion or Propelling Systems Resulting in Lower Carbon Emission

Any improvement of emissions towards lower carbon levels involves an obvious step change in technology for gas turbines. New materials and new materials processing solutions are needed to meet stringent application demands for future products that will provide energy savings, emissions reductions, and other benefits under harsh service conditions (DE-FOA-0002553).

The integrated modeling tool presented contributes to the development of
1. Materials that allow the use of low-carbon, alternative electric-based heating methods or production routes for thermal process loads in reduction, melting, and heating process applications.
2. Materials that allow use of hydrogen or low net carbon fuels in industrial thermal processes.

### 9. For Design of Alloys for Use in Energy Conversion or Propelling Systems Resulting in Reduced Cost of Manufacturing

Selective laser melting, which is a popular powder bed AM process, beats casting, i.e., the conventional manufacturing process for turbine components, in time. But for high volume, casting presently offers superior benefit. The exact numbers depend on the part geometry. Turbine vendors like Siemens have employed casting for manufacturing turbine components for many years, so there is a procedure in place for validating parts for quality. The SLM process, however, is still evolving (Kulkarni 2021).

### 10. For Reducing Defects in Additively Manufactured Alloy Components

The prediction engine, presented in this invention and in the parent invention (U.S. patent application Ser. No. 16/782,829) can be used to optimize the properties of alloys as well as the associated manufacturing processes. The US Navy is interested in developing alloy compositions and manufacturing processes that result in defect reduction in AM components, thus promoting them to be more resistant to fatigue, with potential increases in strength. AM has been applied to cast and wrought alloy compositions in an effort to achieve original alloy properties. Achieving such properties can be difficult because of the varying solidification conditions inherent in different AM processes and part designs. AM alloys have a complex thermal history involving directional heat build-up and repeated melting combined with rapid solidification. AM usually results in a finer microstructure than conventional processing which usually furnishes the AM material better fatigue properties, but debited creep properties. Altering alloy compositions to take advantage of AM solidification variables may result in improved alloy properties (NavySbir211085 2021).

### 12. Further Examples of the Invention

Thus, it will be appreciated by those skilled in the art that the present invention is not restricted to the particular preferred embodiments described with reference to the drawings, and that variations may be made therein without departing from the scope of the invention.

This invention claims:

1. An apparatus for predictive analytics, an apparatus that employs a prediction module, for the purpose of efficiently searching composition space of energetic materials of interest, with energetic materials comprising of propellants, explosives or pyrotechnics, and hence accelerating identification of energetic materials with desired characteristics, an apparatus comprising of a database importing module, for ingesting relevant materials data,
an optional data base abstraction module, also referred to as prediction logic, for abstracting an interface between a prediction module and the database,
a prediction module, also referred to as optimization module, prediction engine or optimization engine, for predicting or optimizing material properties of interest given the materials data ingested, where the prediction module applies a prediction or optimization technique, suitable for the data available and the problem at hand, to a set of descriptors characteristic of energetic materials, where the prediction module is capable of accounting for physics-based models for improved prediction or optimization accuracy, and where the prediction or optimization technique is selected such that the number of unknown parameters in a model employed by the prediction or optimization technique does not exceed the number of data points available for estimating or training the model, and
one or more optional interfaces with, or being integrated into physics-based modeling tools providing calculations of thermo-physical or thermo-chemical properties, first-principle (ab initio) calculations, molecular dynamics simulations or group additivity methods,
an optional reporting and verification module, for reporting and verifying the materials properties estimated,
an apparatus presented either in the form of an integrated or an embedded application.

2. An apparatus according to claim 1, where the prediction engine has the ability to predict specific impulse, density specific impulse, or relative impulse, in case of propellants, but also detonation velocity, detonation pressure, detonation power or volume of gaseous products, in case of explosives or pyrotechnics, by properly accounting for the input sources contributing to variance (uncertainty) in the quantity predicted, and hence decreasing the variance in the quantity predicted.

3. An apparatus according to claim 1, where the prediction engine is capable of accommodating models capturing physical dependencies, referred to as physics-based models, as a priori information, and correspondingly constructing dependencies within the model, for purpose of expediting training of the model and improving prediction accuracy.

4. An apparatus according to claim 1, where the data importing module employs an Export, Transform and Load operation for importing data from a source into the destination prediction database logic, when the destination prediction database logic system represents the data differently from the source.

5. An apparatus according to claim 1, where the data ingested complies with Structured Query Language or JavaScript Object Notation, or is ingested in the form of delimited text, multi-line data, or in the XML, YAML, HTML, KML, JSON, GeoJSON, CSV, Excel, JSON, SQL, DB, DBs or FlatFile formats.

6. An apparatus according to claim 1, where the prediction module can analytically account for combustion temperature in the model, when predicting the specific impulse, density specific impulse or relative specific impulse, for an energetic material, for the purpose of extracting the most from the usually limited input data available.

7. An apparatus according to claim 1, where the prediction module can analytically account for mass density in the model, when predicting the specific impulse, density specific impulse or relative specific impulse, for an energetic material, for the purpose of extracting the most from the usually limited input data available.

8. An apparatus according to claim 1, where the prediction module can analytically account for ratio of specific heats of working fluids in the model, when predicting the specific impulse, density specific impulse or relative specific impulse, for an energetic material, for the purpose of extracting the most from the usually limited input data available.

9. An apparatus according to claim 1, where the inputs to the prediction module comprise of chemical content in stoichiometry, molecular weights of gaseous products (exit gases), freezing, melting and boiling temperatures (temperature range for liquid phase), phase stability, thermodynamic stability of a molecule (thermal stability), reactivity, mass density, thermal coefficients, thermal conductivity, kinematic viscosity, surface tension, heat of formation, heat of reaction, heat of combustion, heat of explosion, temperature of combustion, oxygen balance, oxidizer-to-fuel ratio, ignition, combustion, ignition-delay time, bond dissociation energy, ionization energy, bond energy, other micro-scale (molecular level) properties related to bond length or molecular structure or cohesive energy, taken as a subset or a complete set, and where the outputs comprise of specific impulse, density specific impulse, relative specific impulse, volume of gaseous products, detonation velocity, exhaust velocity, detonation pressure, detonation power, biological toxicity, biocompatibility, flammability, volatility, impact sensitivity or friction sensitivity, again taken as a subset or a complete set.

10. An apparatus according to claim 1, where the descriptors contain encoding of functional groups, comprising of acid groups, alcohol groups, aldehyde groups, alkane groups, amide groups, amine groups, azido groups, ester groups, ether groups, fluoro groups, hydroxy groups, imine groups, ketone groups, methyl groups, nitramines groups, nitrides groups, nitride groups, nitro groups or thiol groups, encoded in the form of a subset or a complete set, for the purpose of capturing the structure, properties or behavior of complex molecules comprising energetic materials in a parametrized form.

11. An apparatus according to claim 1, where the descriptors contain encoding of bond lengths, bond order, bond energies, ionization energies, bond dissociation energies, bond overlap populations, derivatives, substituent groups or aromaticity, encoded in the form of a subset or a complete set, for the purpose of capturing the structure, properties or behavior of complex molecules comprising energetic materials in a parametrized form.

12. An apparatus according to claim 1, where the prediction module is capable of supporting sequential learning, for the purpose of making the most of test data, generated during design of an energetic material, for improved prediction accuracy.

13. An apparatus according to claim 1, where the prediction module supports a hybrid structure, one that combines physics-based modeling with standard machine learning prediction, and where the physics-based modeling comprises calculations of thermo-physical or thermo-chemical properties, first-principle (ab initio) calculations, molecular dynamics simulations or group additivity methods.

14. An apparatus according to claim 1, where the prediction module, also referred to as optimization module, prediction engine or optimization engine, is capable of supporting joint optimization of properties of energetic materials, through a two-step process, where in the first step a neural network are trained separately for estimating or predicting individual properties of energetic materials, or the model coefficients needed for the first step are estimated using multi-variate regression, decision trees, decision tables, support vector machines, Bayesian networks, genetic algorithms, reduced error pruning trees or M5 model trees, but where in the second step Bayesian inference (Bayesian bootstrapping) is employed for maximizing the probability that each of the input properties exceeds a target value (a design criteria) specified.

15. An apparatus according to claim 1, where the prediction module, also referred to as optimization module, prediction engine or optimization engine, is capable of supporting joint optimization of properties of energetic materials, by replacing deterministic coefficients in relations obtained from Kamlet-Jacobs equations by random variables, for purpose of producing a random process, whose unknown parameters are estimated by regression analysis, neural networks, decision trees, decision tables, support vector machines, Bayesian networks, genetic algorithms, reduced error pruning trees or M5 model trees.

16. A method for predictive analytics, one that employs a prediction step, for the purpose of efficiently searching composition space of energetic materials of interest, with energetic materials comprising of propellants, explosives or pyrotechnics, and hence accelerating identification of energetic materials with desired characteristics, a method utilizing
a database importing step, for ingesting the relevant materials data,
a prediction step, also referred to as an optimization step, for predicting or optimizing material properties of interest given the materials data ingested, where the prediction step employs a prediction or optimization technique, suitable for the data available and the problem at hand, to a set of descriptors characteristic of energetic materials, where the prediction step is capable of accounting for physics-based models for improved prediction or optimization accuracy, and where the prediction or optimization technique is selected such that the number of unknown parameters in a model employed by the prediction or optimization technique does not exceed the number of data points available for estimating or training the model, and
one or more optional interface access steps with, for accessing physics-based modeling tools providing calculations of thermo-physical or thermo-chemical properties, first-principle (ab initio) calculations, molecular dynamics simulations or group additivity methods through an application program interface,
an optional reporting and verification step, for reporting and verifying the materials properties estimated.

17. A method according to claim 16, where the prediction or optimization step has the ability to predict specific impulse, density specific impulse, or relative impulse, in case of propellants, but also detonation velocity, detonation pressure, detonation power or volume of gaseous products, in case of explosives or pyrotechnics, by properly accounting for the input sources contributing to variance in the quantity predicted, and hence decreasing the variance (uncertainty) in the quantity predicted.

18. A method according to claim 16, where the prediction or optimization step is capable of accommodating models capturing physical dependencies, referred to as physics-based models, as a priori information, and correspondingly constructing dependencies within the model, for purpose of expediting training of the model and improving prediction accuracy.

19. A method according to claim 16, where the data imported complies with Structured Query Language or JavaScript Object Notation is ingested in the form of delimited text, multi-line data, or in the XML, YAML, HTML, KML, JSON, GeoJSON, CSV, Excel, JSON, SQL, DB, DBs or FlatFile formats.

20. A method according to claim 16, where the prediction or optimization step can analytically account for combustion temperature in the model, when predicting the specific impulse, density specific impulse or relative specific impulse, for an energetic material, for the purpose of extracting the most from the usually limited input data available.

21. A method according to claim 16, where the prediction or optimization step can analytically account for mass density in the model, when predicting the specific impulse, density specific impulse or relative specific impulse, for an energetic material, for the purpose of extracting the most from the usually limited input data available.

22. A method according to claim 16, where the prediction or optimization step can analytically account for ratio of specific heats of working fluids in the model, when predicting the specific impulse, density specific impulse or relative specific impulse, for an energetic material, for the purpose of extracting the most from the usually limited input data available.

23. A method according to claim 16, where the inputs to the prediction or optimization step comprise of chemical content in stoichiometry, molecular weights of gaseous products (exit gases), freezing, melting and boiling temperatures (temperature range for liquid phase), phase stability, thermodynamic stability of a molecule (thermal stability), reactivity, mass density, thermal coefficients, thermal conductivity, kinematic viscosity, surface tension, heat of formation, heat of reaction, heat of combustion, heat of explosion, temperature of combustion, oxygen balance, oxidizer-to-fuel ratio, ignition, combustion, ignition-delay time, bond dissociation energy, ionization energy, bond energy, other micro-scale (molecular level) properties related to bond length or molecular structure or cohesive energy, taken as a subset or a complete set, and where the outputs comprise of specific impulse, density specific impulse, relative specific impulse, volume of gaseous products, detonation velocity, exhaust velocity, detonation pressure, detonation power, biological toxicity, biocompatibility, flammability, volatility, impact sensitivity or friction sensitivity, again taken as a subset or a complete set.

24. A method according to claim 16, where the descriptors contain encoding of functional groups, comprising of acid groups, alcohol groups, aldehyde groups, alkane groups, amide groups, amine groups, azido groups, ester groups, ether groups, fluoro groups, hydroxy groups, imine groups, ketone groups, methyl groups, nitramines groups, nitrides groups, nitride groups, nitro groups or thiol groups, encoded in the form of a subset or a complete set, for the purpose of capturing the structure, properties or behavior of complex molecules comprising energetic materials in a parametrized form.

25. A method according to claim 16, where the descriptors contain encoding of bond lengths, bond order, bond energies, ionization energies, bond dissociation energies, bond overlap populations, derivatives, substituent groups or aromaticity, encoded in the form of a subset or a complete set, for the purpose of capturing the structure, properties or behavior of complex molecules comprising energetic materials in a parametrized form.

26. A method according to claim 16, where the prediction or optimization step is capable of supporting sequential learning, for the purpose of making the most of test data, generated during design of an energetic material, for improved prediction accuracy.

27. A method according to claim 16, where the prediction or optimization step supports a hybrid structure, one that combines physics-based modeling with standard machine learning prediction, and where the physics-based modeling comprises calculations of thermo-physical or thermo-chemical properties, first-principle (ab initio) calculations, molecular dynamics simulations or group additivity methods.

28. A method according to claim 16, where the prediction or optimization step is capable of supporting joint optimization of properties of energetic materials, through a two-step process, where in the first step a neural network are trained separately for estimating or predicting individual properties of energetic materials, or the model coefficients needed for the first step are estimated using multi-variate regression, decision trees, decision tables, support vector machines, Bayesian networks, genetic algorithms, reduced error pruning trees or MS model trees, but where in the second step Bayesian inference (Bayesian bootstrapping) is employed for maximizing the probability that each of the input properties exceeds a target value (a design criteria) specified.

29. A method according to claim 16, where the prediction or optimization step is capable of supporting joint optimization of properties of energetic materials, by replacing deterministic coefficients in relations obtained from Kamlet-Jacobs equations by random variables, for purpose of producing a random process, whose unknown parameters can be estimated by regression analysis, neural networks, decision trees, decision tables, support vector machines, Bayesian networks, genetic algorithms, reduced error pruning trees or M5 model trees.
